SEQUENCE LISTING (1015 sequences)

SEQ ID NO: 1 - RNA, 20 nt, synthetic construct
  misc_feature 1..20: note = Synthetic: Exemplary PCSK9 guide sequence
  source 1..20: mol_type = other RNA
ggtgctagccttgcgttccg

SEQ ID NO: 2 - RNA, 20 nt, synthetic construct
  misc_feature 1..20: note = Synthetic: Exemplary PCSK9 guide sequence
  source 1..20: mol_type = other RNA
gctagccttgcgttccgagg

SEQ ID NO: 3 - RNA, 20 nt, synthetic construct
  misc_feature 1..20: note = Synthetic: Exemplary PCSK9 guide sequence
  source 1..20: mol_type = other RNA
cccgcaccttggcgcagcgg

SEQ ID NO: 4 - RNA, 20 nt, synthetic construct
  misc_feature 1..20: note = Synthetic: Exemplary PCSK9 guide sequence
  source 1..20: mol_type = other RNA
tcttggtgaggtatccccgg

SEQ ID NO: 5 - RNA, 20 nt, synthetic construct
  misc_feature 1..20: note = Synthetic: Exemplary PCSK9 guide sequence
  source 1..20: mol_type = other RNA
gaagatgagtggcgacctgc

SEQ ID NO: 6 - RNA, 20 nt, synthetic construct
  misc_feature 1..20: note = Synthetic: Exemplary PCSK9 guide sequence
  source 1..20: mol_type = other RNA
gtcgacatggggcaacttca

SEQ ID NO: 7 - RNA, 20 nt, synthetic construct
  misc_feature 1..20: note = Synthetic: Exemplary PCSK9 guide sequence
  source 1..20: mol_type = other RNA
gccccatgtcgactacatcg

SEQ ID NO: 8 - RNA, 20 nt, synthetic construct
  misc_feature 1..20: note = Synthetic: Exemplary PCSK9 guide sequence
  source 1..20: mol_type = other RNA
cttggcagttgagcacgcgc

SEQ ID NO: 9 - RNA, 20 nt, synthetic construct
  misc_feature 1..20: note = Synthetic: Exemplary PCSK9 guide sequence
  source 1..20: mol_type = other RNA
cgtgctcaactgccaaggga

SEQ ID NO: 10 - RNA, 20 nt, synthetic construct
  misc_feature 1..20: note = Synthetic: Exemplary PCSK9 guide sequence
  source 1..20: mol_type = other RNA
cctcagcacaggcggcttgt

SEQ ID NO: 11 - RNA, 20 nt, synthetic construct
  misc_feature 1..20: note = Synthetic: Exemplary PCSK9 guide sequence
  source 1..20: mol_type = other RNA
cactggttgggctgacctcg

SEQ ID NO: 12 - RNA, 20 nt, synthetic construct
  misc_feature 1..20: note = Synthetic: Exemplary PCSK9 guide sequence
  source 1..20: mol_type = other RNA
tcctgcgcacgggcgcccgc

SEQ ID NO: 13 - RNA, 20 nt, synthetic construct
  misc_feature 1..20: note = Synthetic: Exemplary PCSK9 guide sequence
  source 1..20: mol_type = other RNA
gttgcctggcacctacgtgg

SEQ ID NO: 14 - RNA, 20 nt, synthetic construct
  misc_feature 1..20: note = Synthetic: Exemplary PCSK9 guide sequence
  source 1..20: mol_type = other RNA
tacccctccacggtaccggg

SEQ ID NO: 15 - RNA, 20 nt, synthetic construct
  misc_feature 1..20: note = Synthetic: Exemplary PCSK9 guide sequence
  source 1..20: mol_type = other RNA
tcatccgcccggtaccgtgg

SEQ ID NO: 16 - RNA, 20 nt, synthetic construct
  misc_feature 1..20: note = Synthetic: Exemplary PCSK9 guide sequence
  source 1..20: mol_type = other RNA
atgtcgccttggaaagacgg

SEQ ID NO: 17 - RNA, 20 nt, synthetic construct
  misc_feature 1..20: note = Synthetic: Exemplary PCSK9 guide sequence
  source 1..20: mol_type = other RNA
gggccatcacttacctatga

SEQ ID NO: 18 - RNA, 20 nt, synthetic construct
  misc_feature 1..20: note = Synthetic: Exemplary PCSK9 guide sequence
  source 1..20: mol_type = other RNA
cagcacactcggggcctaca

SEQ ID NO: 19 - RNA, 20 nt, synthetic construct
  misc_feature 1..20: note = Synthetic: Exemplary PCSK9 guide sequence
  source 1..20: mol_type = other RNA
tgtctacggcgtaggccccc

SEQ ID NO: 20 - RNA, 20 nt, synthetic construct
  misc_feature 1..20: note = Synthetic: Exemplary PCSK9 guide sequence
  source 1..20: mol_type = other RNA
tgcctgtagtgctgacgtcc

SEQ ID NO: 21-100 - skipped (80 entries)

SEQ ID NO: 101 - RNA, 100 nt, synthetic construct
  misc_feature 1..100: note = Synthetic: Exemplary unmodified sgRNA sequence targeting PCSK9
  source 1..100: mol_type = other RNA
ggtgctagccttgcgttccggttttagagctagaaatagcaagttaaaataaggctagtccgttatcaacttgaaaaagtggcaccgagtcggtgctttt

SEQ ID NO: 102 - RNA, 100 nt, synthetic construct
  misc_feature 1..100: note = Synthetic: Exemplary unmodified sgRNA sequence targeting PCSK10
  source 1..100: mol_type = other RNA
gctagccttgcgttccgagggttttagagctagaaatagcaagttaaaataaggctagtccgttatcaacttgaaaaagtggcaccgagtcggtgctttt

SEQ ID NO: 103 - RNA, 100 nt, synthetic construct
  misc_feature 1..100: note = Synthetic: Exemplary unmodified sgRNA sequence targeting PCSK11
  source 1..100: mol_type = other RNA
cccgcaccttggcgcagcgggttttagagctagaaatagcaagttaaaataaggctagtccgttatcaacttgaaaaagtggcaccgagtcggtgctttt

SEQ ID NO: 104 - RNA, 100 nt, synthetic construct
  misc_feature 1..100: note = Synthetic: Exemplary unmodified sgRNA sequence targeting PCSK12
  source 1..100: mol_type = other RNA
tcttggtgaggtatccccgggttttagagctagaaatagcaagttaaaataaggctagtccgttatcaacttgaaaaagtggcaccgagtcggtgctttt

SEQ ID NO: 105 - RNA, 100 nt, synthetic construct
  misc_feature 1..100: note = Synthetic: Exemplary unmodified sgRNA sequence targeting PCSK13
  source 1..100: mol_type = other RNA
gaagatgagtggcgacctgcgttttagagctagaaatagcaagttaaaataaggctagtccgttatcaacttgaaaaagtggcaccgagtcggtgctttt

SEQ ID NO: 106 - RNA, 100 nt, synthetic construct
  misc_feature 1..100: note = Synthetic: Exemplary unmodified sgRNA sequence targeting PCSK14
  source 1..100: mol_type = other RNA
gtcgacatggggcaacttcagttttagagctagaaatagcaagttaaaataaggctagtccgttatcaacttgaaaaagtggcaccgagtcggtgctttt

SEQ ID NO: 107 - RNA, 100 nt, synthetic construct
  misc_feature 1..100: note = Synthetic: Exemplary unmodified sgRNA sequence targeting PCSK15
  source 1..100: mol_type = other RNA
gccccatgtcgactacatcggttttagagctagaaatagcaagttaaaataaggctagtccgttatcaacttgaaaaagtggcaccgagtcggtgctttt

SEQ ID NO: 108 - RNA, 100 nt, synthetic construct
  misc_feature 1..100: note = Synthetic: Exemplary unmodified sgRNA sequence targeting PCSK16
  source 1..100: mol_type = other RNA
cttggcagttgagcacgcgcgttttagagctagaaatagcaagttaaaataaggctagtccgttatcaacttgaaaaagtggcaccgagtcggtgctttt

SEQ ID NO: 109 - RNA, 100 nt, synthetic construct
  misc_feature 1..100: note = Synthetic: Exemplary unmodified sgRNA sequence targeting PCSK17
  source 1..100: mol_type = other RNA
cgtgctcaactgccaagggagttttagagctagaaatagcaagttaaaataaggctagtccgttatcaacttgaaaaagtggcaccgagtcggtgctttt

SEQ ID NO: 110 - RNA, 100 nt, synthetic construct
  misc_feature 1..100: note = Synthetic: Exemplary unmodified sgRNA sequence targeting PCSK18
  source 1..100: mol_type = other RNA
cctcagcacaggcggcttgtgttttagagctagaaatagcaagttaaaataaggctagtccgttatcaacttgaaaaagtggcaccgagtcggtgctttt

SEQ ID NO: 111 - RNA, 100 nt, synthetic construct
  misc_feature 1..100: note = Synthetic: Exemplary unmodified sgRNA sequence targeting PCSK19
  source 1..100: mol_type = other RNA
cactggttgggctgacctcggttttagagctagaaatagcaagttaaaataaggctagtccgttatcaacttgaaaaagtggcaccgagtcggtgctttt

SEQ ID NO: 112 - RNA, 100 nt, synthetic construct
  misc_feature 1..100: note = Synthetic: Exemplary unmodified sgRNA sequence targeting PCSK20
  source 1..100: mol_type = other RNA
tcctgcgcacgggcgcccgcgttttagagctagaaatagcaagttaaaataaggctagtccgttatcaacttgaaaaagtggcaccgagtcggtgctttt

SEQ ID NO: 113 - RNA, 100 nt, synthetic construct
  misc_feature 1..100: note = Synthetic: Exemplary unmodified sgRNA sequence targeting PCSK21
  source 1..100: mol_type = other RNA
gttgcctggcacctacgtgggttttagagctagaaatagcaagttaaaataaggctagtccgttatcaacttgaaaaagtggcaccgagtcggtgctttt

SEQ ID NO: 114 - RNA, 100 nt, synthetic construct
  misc_feature 1..100: note = Synthetic: Exemplary unmodified sgRNA sequence targeting PCSK22
  source 1..100: mol_type = other RNA
tacccctccacggtaccggggttttagagctagaaatagcaagttaaaataaggctagtccgttatcaacttgaaaaagtggcaccgagtcggtgctttt

SEQ ID NO: 115 - RNA, 100 nt, synthetic construct
  misc_feature 1..100: note = Synthetic: Exemplary unmodified sgRNA sequence targeting PCSK23
  source 1..100: mol_type = other RNA
tcatccgcccggtaccgtgggttttagagctagaaatagcaagttaaaataaggctagtccgttatcaacttgaaaaagtggcaccgagtcggtgctttt

SEQ ID NO: 116 - RNA, 100 nt, synthetic construct
  misc_feature 1..100: note = Synthetic: Exemplary unmodified sgRNA sequence targeting PCSK24
  source 1..100: mol_type = other RNA
atgtcgccttggaaagacgggttttagagctagaaatagcaagttaaaataaggctagtccgttatcaacttgaaaaagtggcaccgagtcggtgctttt

SEQ ID NO: 117 - RNA, 100 nt, synthetic construct
  misc_feature 1..100: note = Synthetic: Exemplary unmodified sgRNA sequence targeting PCSK25
  source 1..100: mol_type = other RNA
gggccatcacttacctatgagttttagagctagaaatagcaagttaaaataaggctagtccgttatcaacttgaaaaagtggcaccgagtcggtgctttt

SEQ ID NO: 118 - RNA, 100 nt, synthetic construct
  misc_feature 1..100: note = Synthetic: Exemplary unmodified sgRNA sequence targeting PCSK26
  source 1..100: mol_type = other RNA
cagcacactcggggcctacagttttagagctagaaatagcaagttaaaataaggctagtccgttatcaacttgaaaaagtggcaccgagtcggtgctttt

SEQ ID NO: 119 - RNA, 100 nt, synthetic construct
  misc_feature 1..100: note = Synthetic: Exemplary unmodified sgRNA sequence targeting PCSK27
  source 1..100: mol_type = other RNA
tgtctacggcgtaggcccccgttttagagctagaaatagcaagttaaaataaggctagtccgttatcaacttgaaaaagtggcaccgagtcggtgctttt

SEQ ID NO: 120 - RNA, 100 nt, synthetic construct
  misc_feature 1..100: note = Synthetic: Exemplary unmodified sgRNA sequence targeting PCSK28
  source 1..100: mol_type = other RNA
tgcctgtagtgctgacgtccgttttagagctagaaatagcaagttaaaataaggctagtccgttatcaacttgaaaaagtggcaccgagtcggtgctttt

SEQ ID NO: 121 - RNA, 91 nt, synthetic construct
  misc_feature 1..91: note = Synthetic: Exemplary unmodified sgRNA sequence targeting PCSK29
  source 1..91: mol_type = other RNA
cgtgctcaactgccaagggagttttagagctagaaatagcaagttaaaataaggctagtccgttatcacgaaagggcaccgagtcggtgct

SEQ ID NO: 122 - RNA, 91 nt, synthetic construct
  misc_feature 1..91: note = Synthetic: Exemplary unmodified sgRNA sequence targeting PCSK30
  source 1..91: mol_type = other RNA
tacccctccacggtaccggggttttagagctagaaatagcaagttaaaataaggctagtccgttatcacgaaagggcaccgagtcggtgct

SEQ ID NO: 123 - RNA, 91 nt, synthetic construct
  misc_feature 1..91: note = Synthetic: Exemplary unmodified sgRNA sequence targeting PCSK31
  source 1..91: mol_type = other RNA
cagcacactcggggcctacagttttagagctagaaatagcaagttaaaataaggctagtccgttatcacgaaagggcaccgagtcggtgct

SEQ ID NO: 124-200 - skipped (77 entries)

SEQ ID NO: 201 - RNA, 100 nt, synthetic construct; identical to SEQ ID NO: 101
  misc_feature 1..100: note = Synthetic: Exemplary modified sgRNA sequence targeting PCSK9
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 202 - RNA, 100 nt, synthetic construct; identical to SEQ ID NO: 102
  misc_feature 1..100: note = Synthetic: Exemplary modified sgRNA sequence targeting PCSK10
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 203 - RNA, 100 nt, synthetic construct; identical to SEQ ID NO: 103
  misc_feature 1..100: note = Synthetic: Exemplary modified sgRNA sequence targeting PCSK11
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 204 - RNA, 100 nt, synthetic construct; identical to SEQ ID NO: 104
  misc_feature 1..100: note = Synthetic: Exemplary modified sgRNA sequence targeting PCSK12
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 205 - RNA, 100 nt, synthetic construct; identical to SEQ ID NO: 105
  misc_feature 1..100: note = Synthetic: Exemplary modified sgRNA sequence targeting PCSK13
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 206 - RNA, 100 nt, synthetic construct; identical to SEQ ID NO: 106
  misc_feature 1..100: note = Synthetic: Exemplary modified sgRNA sequence targeting PCSK14
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 207 - RNA, 100 nt, synthetic construct; identical to SEQ ID NO: 107
  misc_feature 1..100: note = Synthetic: Exemplary modified sgRNA sequence targeting PCSK15
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 208 - RNA, 100 nt, synthetic construct; identical to SEQ ID NO: 108
  misc_feature 1..100: note = Synthetic: Exemplary modified sgRNA sequence targeting PCSK16
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 209 - RNA, 100 nt, synthetic construct; identical to SEQ ID NO: 109
  misc_feature 1..100: note = Synthetic: Exemplary modified sgRNA sequence targeting PCSK17
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 210 - RNA, 100 nt, synthetic construct; identical to SEQ ID NO: 110
  misc_feature 1..100: note = Synthetic: Exemplary modified sgRNA sequence targeting PCSK18
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 211 - RNA, 100 nt, synthetic construct; identical to SEQ ID NO: 111
  misc_feature 1..100: note = Synthetic: Exemplary modified sgRNA sequence targeting PCSK19
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 212 - RNA, 100 nt, synthetic construct; identical to SEQ ID NO: 112
  misc_feature 1..100: note = Synthetic: Exemplary modified sgRNA sequence targeting PCSK20
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 213 - RNA, 100 nt, synthetic construct; identical to SEQ ID NO: 113
  misc_feature 1..100: note = Synthetic: Exemplary modified sgRNA sequence targeting PCSK21
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 214 - RNA, 100 nt, synthetic construct; identical to SEQ ID NO: 114
  misc_feature 1..100: note = Synthetic: Exemplary modified sgRNA sequence targeting PCSK22
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 215 - RNA, 100 nt, synthetic construct; identical to SEQ ID NO: 115
  misc_feature 1..100: note = Synthetic: Exemplary modified sgRNA sequence targeting PCSK23
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 216 - RNA, 100 nt, synthetic construct; identical to SEQ ID NO: 116
  misc_feature 1..100: note = Synthetic: Exemplary modified sgRNA sequence targeting PCSK24
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 217 - RNA, 100 nt, synthetic construct; identical to SEQ ID NO: 117
  misc_feature 1..100: note = Synthetic: Exemplary modified sgRNA sequence targeting PCSK25
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 218 - RNA, 100 nt, synthetic construct; identical to SEQ ID NO: 118
  misc_feature 1..100: note = Synthetic: Exemplary modified sgRNA sequence targeting PCSK26
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 219 - RNA, 100 nt, synthetic construct; identical to SEQ ID NO: 119
  misc_feature 1..100: note = Synthetic: Exemplary modified sgRNA sequence targeting PCSK27
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 220 - RNA, 100 nt, synthetic construct; identical to SEQ ID NO: 120
  misc_feature 1..100: note = Synthetic: Exemplary modified sgRNA sequence targeting PCSK28
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 221 - RNA, 91 nt, synthetic construct; identical to SEQ ID NO: 121
  misc_feature 1..91: note = Synthetic: Exemplary modified sgRNA sequence targeting PCSK29
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 88..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 222 - RNA, 91 nt, synthetic construct; identical to SEQ ID NO: 122
  misc_feature 1..91: note = Synthetic: Exemplary modified sgRNA sequence targeting PCSK30
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 88..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 223 - RNA, 91 nt, synthetic construct; identical to SEQ ID NO: 123
  misc_feature 1..91: note = Synthetic: Exemplary modified sgRNA sequence targeting PCSK31
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 88..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 224-300 - skipped (77 entries)

SEQ ID NO: 301 - RNA, 22 nt, synthetic construct
  misc_feature 1..22: note = Synthetic: Exemplary unmodified SpyCas9 scaffold sequence
  source 1..22: mol_type = other RNA
gttttagagctatgctgttttg

SEQ ID NO: 302 - RNA, 80 nt, synthetic construct
  misc_feature 1..80: note = Synthetic: Exemplary unmodified SpyCas9 scaffold sequence
  source 1..80: mol_type = other RNA
gttttagagctagaaatagcaagttaaaataaggctagtccgttatcaacttgaaaaagtggcaccgagtcggtgctttt

SEQ ID NO: 303 - RNA, 76 nt, synthetic construct
  misc_feature 1..76: note = Synthetic: Exemplary unmodified SpyCas9 scaffold sequence
  source 1..76: mol_type = other RNA
gttttagagctagaaatagcaagttaaaataaggctagtccgttatcaacttgaaaaagtggcaccgagtcggtgc

SEQ ID NO: 304 - RNA, 77 nt, synthetic construct
  misc_feature 1..77: note = Synthetic: Exemplary unmodified SpyCas9 scaffold sequence
  source 1..77: mol_type = other RNA
gttttagagctagaaatagcaagttaaaataaggctagtccgttatcaacttgaaaaagtggcaccgagtcggtgct

SEQ ID NO: 305 - RNA, 68 nt, synthetic construct
  misc_feature 1..68: note = Synthetic: Exemplary unmodified SpyCas9 scaffold sequence
  source 1..68: mol_type = other RNA
gttttagagctagaaatagcaagttaaaataaggctagtccgttatcaacttggcaccgagtcggtgc

SEQ ID NO: 306 - RNA, 68 nt, synthetic construct
  misc_feature 1..68: note = Synthetic: Exemplary unmodified SpyCas9 scaffold sequence
  source 1..68: mol_type = other RNA
gttttagagctagaaatagcaagttaaaataaggctagtccgttatcaaaatggcaccgagtcggtgc

SEQ ID NO: 307 - RNA, 69 nt, synthetic construct
  misc_feature 1..69: note = Synthetic: Exemplary unmodified SpyCas9 scaffold sequence
  source 1..69: mol_type = other RNA
ggttttagagctagaaatagcaagttaaaataaggctagtccgttatcaaaatggcaccgagtcggtgc

SEQ ID NO: 308 - RNA, 70 nt, synthetic construct
  misc_feature 1..70: note = Synthetic: Exemplary unmodified SpyCas9 scaffold sequence
  source 1..70: mol_type = other RNA
gttttagagctagaaatagcaagttaaaataaggctagtccgttatcacgaaagggcaccgagtcggtgc

SEQ ID NO: 309 - RNA, 71 nt, synthetic construct
  misc_feature 1..71: note = Synthetic: Exemplary unmodified SpyCas9 scaffold sequence
  source 1..71: mol_type = other RNA
gttttagagctagaaatagcaagttaaaataaggctagtccgttatcacgaaagggcaccgagtcggtgct

SEQ ID NO: 310-400 - skipped (91 entries)

SEQ ID NO: 401 - RNA, 100 nt, synthetic construct
  misc_feature 1..100: note = Synthetic: Exemplary unmodified SpyCas9 guide RNA sequence
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 402 - RNA, 96 nt, synthetic construct
  misc_feature 1..96: note = Synthetic: Exemplary unmodified SpyCas9 guide RNA sequence
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 403 - RNA, 97 nt, synthetic construct
  misc_feature 1..97: note = Synthetic: Exemplary unmodified SpyCas9 guide RNA sequence
  source 1..97: mol_type = other RNA
nnnnnnnnnnnnnnnnnnnngttttagagctagaaatagcaagttaaaataaggctagtccgttatcaacttgaaaaagtggcaccgagtcggtgct

SEQ ID NO: 404 - RNA, 88 nt, synthetic construct
  misc_feature 1..88: note = Synthetic: Exemplary unmodified SpyCas9 guide RNA sequence
  source 1..88: mol_type = other RNA
nnnnnnnnnnnnnnnnnnnngttttagagctagaaatagcaagttaaaataaggctagtccgttatcaacttggcaccgagtcggtgc

SEQ ID NO: 405 - RNA, 88 nt, synthetic construct
  misc_feature 1..88: note = Synthetic: Exemplary unmodified SpyCas9 guide RNA sequence
  source 1..88: mol_type = other RNA
nnnnnnnnnnnnnnnnnnnngttttagagctagaaatagcaagttaaaataaggctagtccgttatcaaaatggcaccgagtcggtgc

SEQ ID NO: 406 - RNA, 89 nt, synthetic construct
  misc_feature 1..89: note = Synthetic: Exemplary unmodified SpyCas9 guide RNA sequence
  source 1..89: mol_type = other RNA
nnnnnnnnnnnnnnnnnnnnggttttagagctagaaatagcaagttaaaataaggctagtccgttatcaaaatggcaccgagtcggtgc

SEQ ID NO: 407 - RNA, 90 nt, synthetic construct
  misc_feature 1..90: note = Synthetic: Exemplary unmodified SpyCas9 guide RNA sequence
  source 1..90: mol_type = other RNA
nnnnnnnnnnnnnnnnnnnngttttagagctagaaatagcaagttaaaataaggctagtccgttatcacgaaagggcaccgagtcggtgc

SEQ ID NO: 408 - RNA, 91 nt, synthetic construct
  misc_feature 1..91: note = Synthetic: Exemplary unmodified SpyCas9 guide RNA sequence
  source 1..91: mol_type = other RNA
nnnnnnnnnnnnnnnnnnnngttttagagctagaaatagcaagttaaaataaggctagtccgttatcacgaaagggcaccgagtcggtgct

SEQ ID NO: 409-500 - skipped (92 entries)

SEQ ID NO: 501 - RNA, 80 nt, synthetic construct; identical to SEQ ID NO: 302
  misc_feature 1..80: note = Synthetic: Exemplary modified SpyCas9 guide scaffold sequence
  modified_base 9..20: mod_base = OTHER; note = 2'-O-methyl modified nucleotide
  modified_base 49..80: mod_base = OTHER; note = 2'-O-methyl modified nucleotide
  modified_base 77..80: mod_base = OTHER; note = phosphorothioate linkage
  source 1..80: mol_type = other RNA

SEQ ID NO: 502 - RNA, 76 nt, synthetic construct; identical to SEQ ID NO: 303
  misc_feature 1..76: note = Synthetic: Exemplary modified SpyCas9 guide scaffold sequence
  modified_base 9..20: mod_base = OTHER; note = 2'-O-methyl modified nucleotide
  modified_base 49..76: mod_base = OTHER; note = 2'-O-methyl modified nucleotide
  source 1..76: mol_type = other RNA

SEQ ID NO: 503 - RNA, 77 nt, synthetic construct; identical to SEQ ID NO: 304
  misc_feature 1..77: note = Synthetic: Exemplary modified SpyCas9 guide scaffold sequence
  modified_base 9..20: mod_base = OTHER; note = 2'-O-methyl modified nucleotide
  modified_base 49..77: mod_base = OTHER; note = 2'-O-methyl modified nucleotide
  source 1..77: mol_type = other RNA

SEQ ID NO: 504 - RNA, 68 nt, synthetic construct; identical to SEQ ID NO: 305
  misc_feature 1..68: note = Synthetic: Exemplary modified SpyCas9 guide scaffold sequence
  modified_base 9..20: mod_base = OTHER; note = 2'-O-methyl modified nucleotide
  modified_base 66..68: mod_base = OTHER; note = 2'-O-methyl modified nucleotide
  modified_base 65..68: mod_base = OTHER; note = phosphorothioate linkage
  source 1..68: mol_type = other RNA

SEQ ID NO: 505 - RNA, 68 nt, synthetic construct; identical to SEQ ID NO: 306
  misc_feature 1..68: note = Synthetic: Exemplary modified SpyCas9 guide scaffold sequence
  modified_base 9..20: mod_base = OTHER; note = 2'-O-methyl modified nucleotide
  modified_base 66..68: mod_base = OTHER; note = 2'-O-methyl modified nucleotide
  modified_base 65..68: mod_base = OTHER; note = phosphorothioate linkage
  source 1..68: mol_type = other RNA

SEQ ID NO: 506 - RNA, 68 nt, synthetic construct; identical to SEQ ID NO: 306
  misc_feature 1..68: note = Synthetic: Exemplary modified SpyCas9 guide scaffold sequence
  modified_base 9..20: mod_base = OTHER; note = 2'-O-methyl modified nucleotide
  modified_base 49..68: mod_base = OTHER; note = 2'-O-methyl modified nucleotide
  modified_base 65..68: mod_base = OTHER; note = phosphorothioate linkage
  source 1..68: mol_type = other RNA

SEQ ID NO: 507 - RNA, 70 nt, synthetic construct; identical to SEQ ID NO: 308
  misc_feature 1..70: note = Synthetic: Exemplary modified SpyCas9 guide scaffold sequence
  modified_base 9..20: mod_base = OTHER; note = 2'-O-methyl modified nucleotide
  modified_base 68..70: mod_base = OTHER; note = 2'-O-methyl modified nucleotide
  modified_base 67..70: mod_base = OTHER; note = phosphorothioate linkage
  source 1..70: mol_type = other RNA

SEQ ID NO: 508 - RNA, 70 nt, synthetic construct; identical to SEQ ID NO: 308
  misc_feature 1..70: note = Synthetic: Exemplary modified SpyCas9 guide scaffold sequence
  modified_base 9..20: mod_base = OTHER; note = 2'-O-methyl modified nucleotide
  modified_base 49..70: mod_base = OTHER; note = 2'-O-methyl modified nucleotide
  modified_base 67..70: mod_base = OTHER; note = phosphorothioate linkage
  source 1..70: mol_type = other RNA

SEQ ID NO: 509 - RNA, 71 nt, synthetic construct; identical to SEQ ID NO: 309
  misc_feature 1..71: note = Synthetic: Exemplary modified SpyCas9 guide scaffold sequence
  modified_base 9..20: mod_base = OTHER; note = 2'-O-methyl modified nucleotide
  modified_base 69..71: mod_base = OTHER; note = 2'-O-methyl modified nucleotide
  modified_base 68..71: mod_base = OTHER; note = phosphorothioate linkage
  source 1..71: mol_type = other RNA

SEQ ID NO: 510 - RNA, 71 nt, synthetic construct; identical to SEQ ID NO: 309
  misc_feature 1..71: note = Synthetic: Exemplary modified SpyCas9 guide scaffold sequence
  modified_base 9..20: mod_base = OTHER; note = 2'-O-methyl modified nucleotide
  modified_base 68..71: mod_base = OTHER; note = 2'-O-methyl modified nucleotide
  modified_base 70..71: mod_base = OTHER; note = phosphorothioate linkage
  source 1..71: mol_type = other RNA

SEQ ID NO: 511 - RNA, 71 nt, synthetic construct; identical to SEQ ID NO: 309
  misc_feature 1..71: note = Synthetic: Exemplary modified SpyCas9 guide scaffold sequence
  modified_base 5: mod_base = OTHER; note = 2'-fluoro modified nucleotide
  modified_base 8..22: mod_base = OTHER; note = 2'-O-methyl modified nucleotide
  modified_base 25: mod_base = OTHER; note = 2'-fluoro modified nucleotide
  modified_base 26: mod_base = OTHER; note = 2'-O-methyl modified nucleotide
  modified_base 27: mod_base = OTHER; note = 2'-fluoro modified nucleotide
  modified_base 28: mod_base = OTHER; note = 2'-O-methyl modified nucleotide
  modified_base 29: mod_base = OTHER; note = 2'-fluoro modified nucleotide
  modified_base 30: mod_base = OTHER; note = 2'-O-methyl modified nucleotide
  modified_base 32..37: mod_base = OTHER; note = 2'-O-methyl modified nucleotide
  modified_base 40..41: mod_base = OTHER; note = 2'-O-methyl modified nucleotide
  modified_base 44: mod_base = OTHER; note = 2'-fluoro modified nucleotide
  modified_base 46..47: mod_base = OTHER; note = 2'-O-methyl modified nucleotide
  modified_base 49..71: mod_base = OTHER; note = 2'-O-methyl modified nucleotide
  modified_base 68..71: mod_base = OTHER; note = phosphorothioate linkage
  source 1..71: mol_type = other RNA

SEQ ID NO: 512 - RNA, 71 nt, synthetic construct; identical to SEQ ID NO: 309
  misc_feature 1..71: note = Synthetic: Exemplary modified SpyCas9 guide scaffold sequence
  modified_base 9..20: mod_base = OTHER; note = 2'-O-methyl modified nucleotide
  modified_base 68..71: mod_base = OTHER; note = 2'-O-methyl modified nucleotide
  modified_base 67..71: mod_base = OTHER; note = phosphorothioate linkage
  source 1..71: mol_type = other RNA

SEQ ID NO: 513-600 - skipped (88 entries)

SEQ ID NO: 601 - RNA, 100 nt, synthetic construct; identical to SEQ ID NO: 401
  misc_feature 1..100: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA

SEQ ID NO: 602 - RNA, 96 nt, synthetic construct; identical to SEQ ID NO: 402
  misc_feature 1..96: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 69..96: mod_base = OTHER; note = 2'-O-methyl modified nucleotide
  source 1..96: mol_type = other RNA

SEQ ID NO: 603 - RNA, 97 nt, synthetic construct; identical to SEQ ID NO: 403
  misc_feature 1..97: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 69..97: mod_base = OTHER; note = 2'-O-methyl modified nucleotide
  source 1..97: mol_type = other RNA

SEQ ID NO: 604 - RNA, 88 nt, synthetic construct; identical to SEQ ID NO: 404
  misc_feature 1..88: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 86..88: mod_base = OTHER; note = 2'-O-methyl modified nucleotide
  modified_base 85..88: mod_base = OTHER; note = phosphorothioate linkage
  source 1..88: mol_type = other RNA

SEQ ID NO: 605 - RNA, 88 nt, synthetic construct; identical to SEQ ID NO: 405
  misc_feature 1..88: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 86..88: mod_base = OTHER; note = 2'-O-methyl modified nucleotide
  modified_base 85..88: mod_base = OTHER; note = phosphorothioate linkage
  source 1..88: mol_type = other RNA

SEQ ID NO: 606 - RNA, 88 nt, synthetic construct; identical to SEQ ID NO: 405
  misc_feature 1..88: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 69..88: mod_base = OTHER; note = 2'-O-methyl modified nucleotide
  modified_base 85..88: mod_base = OTHER; note = phosphorothioate linkage
  source 1..88: mol_type = other RNA

SEQ ID NO: 607 - RNA, 90 nt, synthetic construct; identical to SEQ ID NO: 407
  misc_feature 1..90: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 88..90: mod_base = OTHER; note = 2'-O-methyl modified nucleotide
  modified_base 87..90: mod_base = OTHER; note = phosphorothioate linkage
  source 1..90: mol_type = other RNA

SEQ ID NO: 608 - RNA, 90 nt, synthetic construct; identical to SEQ ID NO: 407
  misc_feature 1..90: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 69..90: mod_base = OTHER; note = 2'-O-methyl modified nucleotide
  modified_base 87..90: mod_base = OTHER; note = phosphorothioate linkage
  source 1..90: mol_type = other RNA

SEQ ID NO: 609 - RNA, 91 nt, synthetic construct; identical to SEQ ID NO: 408
  misc_feature 1..91: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 89..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 610 - RNA, 91 nt, synthetic construct; identical to SEQ ID NO: 408
  misc_feature 1..91: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 88..91: mod_base = OTHER; note = 2'-O-methyl modified nucleotide
  modified_base 90..91: mod_base = OTHER; note = phosphorothioate linkage
  source 1..91: mol_type = other RNA

SEQ ID NO: 611 - RNA, 91 nt, synthetic construct; identical to SEQ ID NO: 408
  misc_feature 1..91: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 25: mod_base = OTHER; note = 2'-fluoro modified nucleotide
  modified_base 28..42: mod_base = OTHER; note = 2'-O-methyl modified nucleotide
  modified_base 45: mod_base = OTHER; note = 2'-fluoro modified nucleotide
  modified_base 46: mod_base = OTHER; note = 2'-O-methyl modified nucleotide
  modified_base 47: mod_base = OTHER; note = 2'-fluoro modified nucleotide
  modified_base 48: mod_base = OTHER; note = 2'-O-methyl modified nucleotide
  modified_base 49: mod_base = OTHER; note = 2'-fluoro modified nucleotide
  modified_base 50: mod_base = OTHER; note = 2'-O-methyl modified nucleotide
  modified_base 52..57: mod_base = OTHER; note = 2'-O-methyl modified nucleotide
  modified_base 61: mod_base = OTHER; note = 2'-O-methyl modified nucleotide
  modified_base 64: mod_base = OTHER; note = 2'-fluoro modified nucleotide
  modified_base 66..67: mod_base = OTHER; note = 2'-O-methyl modified nucleotide
  modified_base 69..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 612 - RNA, 91 nt, synthetic construct; identical to SEQ ID NO: 408
  misc_feature 1..91: note = Synthetic: Exemplary modified SpyCas9 guide sequence
  modified_base 1..3: mod_base = OTHER; note = 2'-O-methyl modified nucleotide
  modified_base 1..4: mod_base = OTHER; note = phosphorothioate linkage
  modified_base 29..40: mod_base = OTHER; note = 2'-O-methyl modified nucleotide
  modified_base 88..91: mod_base = OTHER; note = 2'-O-methyl modified nucleotide
  modified_base 88..91: mod_base = OTHER; note = phosphorothioate linkage
  source 1..91: mol_type = other RNA

SEQ ID NO: 613-1000 - skipped (388 entries)

SEQ ID NO: 1001 - protein, 1379 aa, synthetic construct
  REGION 1..1379: note = Synthetic: Cas9 amino acid sequence with 1x NLS (RNP)
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 1002 - RNA, 4423 nt, synthetic construct
  misc_feature 1..4423: note = Synthetic: mRNA encoding Spy Cas9
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 1003 - RNA, 4140 nt, synthetic construct
  misc_feature 1..4140: note = Synthetic: Open reading frame for Spy Cas9
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 1004 - protein, 1379 aa, synthetic construct; identical to SEQ ID NO: 1001
  REGION 1..1379: note = Synthetic: Amino acid sequence for Spy Cas9

SEQ ID NO: 1005 - RNA, 4516 nt, synthetic construct
  misc_feature 1..4516: note = Synthetic: mRNA encoding Spy Cas9
  source 1..4516: mol_type = other RNA
gggtcccgcagtcggcgtccagcggctctgcttgttcgtgtgtgtgtcgttgcaggccttattcggatccgccaccatggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 1006 - RNA, 4140 nt, synthetic construct
  misc_feature 1..4140: note = Synthetic: Open reading frame for Spy Cas9
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 1007 - protein, 1379 aa, synthetic construct; identical to SEQ ID NO: 1001
  REGION 1..1379: note = Synthetic: Amino acid sequence for Spy Cas9

SEQ ID NO: 1008 - RNA, 4480 nt, synthetic construct
  misc_feature 1..4480: note = Synthetic: mRNA encoding Spy Cas9 with HiBiT tag
  source 1..4480: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 1009 - RNA, 4197 nt, synthetic construct
  misc_feature 1..4197: note = Synthetic: Open reading frame for Spy Cas9 with HiBiT tag
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 1010 - protein, 1398 aa, synthetic construct
  REGION 1..1398: note = Synthetic: Amino acid sequence for SpyCas9 with HiBiT tag
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 1011 - RNA, 100 nt, synthetic construct
  misc_feature 1..100: note = Synthetic: Exemplary EMX1 guide RNA (G000644)
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 1012 - RNA, 100 nt, synthetic construct
  misc_feature 1..100: note = Synthetic: Exemplary VEGFA guide RNA (G000645)
  modified_base 1..4: mod_base = OTHER; note = phosphorothioate linkage
  modified_base 1..3: mod_base = OTHER; note = 2'-O-methyl modified nucleotide
  modified_base 29..40: mod_base = OTHER; note = 2'-O-methyl modified nucleotide
  modified_base 69..100: mod_base = OTHER; note = 2'-O-methyl modified nucleotide
  modified_base 97..100: mod_base = OTHER; note = phosphorothioate linkage
  source 1..100: mol_type = other RNA
gaccccctccaccccgcctcgttttagagctagaaatagcaagttaaaataaggctagtccgttatcaacttgaaaaagtggcaccgagtcggtgctttt

SEQ ID NO: 1013 - protein, 7 aa, synthetic construct
  REGION 1..7: note = Synthetic: Exemplary SV40 NLS
PKKKRKV

SEQ ID NO: 1014 - protein, 7 aa, synthetic construct
  REGION 1..7: note = Synthetic: Exemplary SV40 NLS
PKKKRRV

SEQ ID NO: 1015 - protein, 16 aa, synthetic construct
  REGION 1..16: note = Synthetic: Exemplary nucleoplasmin NLS
KRPAATKKAGQAKKKK

(19) United States
(12) Patent Application Publication
(10) Pub. No.: US 2025/0313845 A1
(43) Pub. Date: Oct. 9, 2025

(54) Compositions and Methods for Proprotein Convertase Subtilisin Kexin 9 (PCSK9) Editing

(71) Applicant: Intellia Therapeutics, Inc., Cambridge, MA (US)

(72) Inventors: Gamze Karaca, Cambridge, MA (US); Trisha Tirthankar Das, Dallas, TX (US); Arti Mahendra Prakash Kanjolia, Malden, MA (US); Hayley Maney, Woburn, MA (US)

(73) Assignee: Intellia Therapeutics, Inc., Cambridge, MA (US)

(21) Appl. No.: 19/242,255

(22) Filed: Jun. 18, 2025

Related U.S. Application Data
(63) Continuation of application No. PCT/US23/85042, filed on Dec. 20, 2023.
(60) Provisional application No. 63/434,394, filed on Dec. 21, 2022.

(51) Int. Cl. C12N 15/113; A61K 38/46; A61K 48/00; C12N 9/22; C12N 15/88

(52) U.S. Cl. CPC C12N 15/1137; A61K 38/465; A61K 48/005; C12N 9/226; C12N 15/88; C12N 2310/20; C12N 2800/80

(57) ABSTRACT

The present disclosure provides compositions and methods for modifying a PCSK9 gene. In some aspects, the present disclosure provides a guide RNA, compositions thereof, and pharmaceutical compositions comprising a guide RNA or a composition as described herein. In some aspects, the present disclosure also provides uses and methods of using a guide RNA, a composition thereof, or a pharmaceutical composition as described herein, for inducing a double-strand break or a single-strand break in a PCSK9 gene, for reducing expression of a PCSK9 gene in a cell or subject, and for treating a patient having or at risk of having a PCSK9-related disease or condition.

### CROSS-REFERENCE TO RELATED APPLICATIONS

This application is a continuation application of International Application No. PCT/US2023/085042, filed Dec. 20, 2023, which claims the benefit of priority to U.S. Provisional Application No. 63/434,394, filed Dec. 21, 2022, which is herein incorporated by reference in its entirety.

### REFERENCE TO ELECTRONIC SEQUENCE LISTING

The application contains a Sequence Listing which has been submitted electronically in .XML format and is hereby incorporated by reference in its entirety. Said. XML copy, created on Nov. 28, 2023, is named “01155-0061-00PCT.xml” and is 508,922 bytes in size. The sequence listing contained in this. XML file is part of the specification and is hereby incorporated by reference herein in its entirety.

### INTRODUCTION AND SUMMARY

Proprotein Convertase Subtilisin Kexin 9 (PCSK9) is a member of the subtilisin serine protease family, expressed in the liver, intestine and kidney tissues. It is a key regulator of circulating low-density lipoprotein (LDL) cholesterol levels and plays a role in cholesterol and fatty acid metabolism. PCSK9 has been shown to induce LDL receptor degradation, in particular in the liver, thereby increasing circulating LDL cholesterol levels in the blood.

Excess production of the PCSK9 protein, or mutations in the PCSK9 gene, have been demonstrated to significantly affect total cholesterol and LDL cholesterol in the general population, and have been associated with cardiovascular diseases (e.g., autosomal dominant familial hypercholesterolemia) and chronic liver injury.

The present disclosure provides compositions and methods for modifying a PCSK9 gene. In some aspects, the present disclosure provides a guide RNA, compositions thereof, and pharmaceutical compositions comprising a guide RNA or a composition as described herein. In some aspects, the present disclosure also provides uses and methods of using a guide RNA, a composition thereof, or a pharmaceutical composition as described herein, for inducing a double-strand break or a single-strand break in a PCSK9 gene, for reducing expression of a PCSK9 gene in a cell or subject, and for treating a patient having or at risk of having a PCSK9-related disease or condition. In some aspects, the present disclosure also provides uses and methods of using a guide RNA, a composition thereof, or a pharmaceutical composition as described herein, for inducing a double-strand break in a PCSK9 gene, for reducing expression of a PCSK9 gene in a cell or subject, and for treating a patient having or at risk of having a PCSK9-related disease or condition.

In some embodiments, the guide RNA comprises a guide region and a conserved region. In some embodiments, the guide RNA comprises a nucleotide sequence targeting a locus of a PCSK9 gene. In some embodiments, the guide RNA is a modified guide RNA.

In some aspects, the present disclosure provides a composition comprising a guide RNA as described herein. In some embodiments, the composition further comprises an RNA-guided DNA binding agent, i.e., a polypeptide RNA-guided DNA binding agent or a nucleic acid encoding an RNA-guided DNA binding agent. In some embodiments, the nucleic acid encoding an RNA-guided DNA binding agent comprises an mRNA comprising an open reading frame (ORF) encoding an RNA-guided DNA binding agent. In some embodiments, the RNA-guided DNA binding agent is a Cas9 nuclease. In some embodiments, the Cas9 is S. pyogenes (“Spy”) Cas9. In some embodiments, the Cas9 is a SpyCas9 cleavase.

In some embodiments, a composition as described herein further comprises a pharmaceutical excipient. In some embodiments, the guide RNA comprised in the composition is associated with a lipid nanoparticle (LNP). In some embodiments, the LNP comprises a cationic lipid. In some embodiments, the LNP comprises a helper lipid. In some embodiments, the helper lipid is cholesterol. In some embodiments, the LNP comprises a neutral lipid. In some embodiments, the neutral lipid is 1,2-distearoyl-sn-glycero-3-phosphocholine (DSPC). In some embodiments, the LNP comprises a stealth lipid. In some embodiments, the stealth lipid is 1,2-dimyristoyl-rac-glycero-3-methoxypolyethylene glycol-2000 (PEG2k-DMG).

In some aspects, the present disclosure provides a pharmaceutical composition. In some embodiments, the pharmaceutical composition comprises a guide RNA as described herein, or a composition as described herein. In some embodiments, the pharmaceutical composition comprises a composition as described herein comprising a guide RNA as described herein, e.g., a modified guide RNA, and a SpyCas9 cleavase.

In some aspects, the present disclosure provides use of a guide RNA as described herein, or a composition as described herein, for inducing a double-strand break or a single-strand break within a PCSK9 gene in a cell. In some aspects, the present disclosure provides a pharmaceutical composition comprising a guide RNA as described herein, or a composition as described herein, for inducing a double-strand break or a single-strand break within a PCSK9 gene in a cell. In some embodiments, the cell is in a subject. In some aspects, the present disclosure provides use of a guide RNA as described herein, or a composition as described herein, for reducing expression of a PCSK9 gene in a cell or subject. In some aspects, the present disclosure provides a pharmaceutical composition comprising a guide RNA as described herein, or a composition as described herein, for reducing expression of a PCSK9 gene in a cell or subject. In some embodiments, the cell is in a subject.

In some aspects, the present disclosure provides use of a guide RNA as described herein, or a composition as described herein, for inducing a double-strand break within a PCSK9 gene in a cell. In some aspects, the present disclosure provides a pharmaceutical composition comprising a guide RNA as described herein, or a composition as described herein, for inducing a double-strand break within a PCSK9 gene in a cell. In some embodiments, the cell is in a subject. In some aspects, the present disclosure provides use of a guide RNA as described herein, or a composition as described herein, for inducing a double-strand break within a PCSK9 gene in a cell for reducing expression of a PCSK9 gene in a cell or subject. In some aspects, the present disclosure provides a pharmaceutical composition comprising a guide RNA as described herein, or a composition as described herein, for inducing a double-strand break within a PCSK9 gene in a cell for reducing expression of a PCSK9 gene in a cell or subject. In some embodiments, the cell is in a subject.

In some aspects, the present disclosure provides use of a guide RNA as described herein, or a composition as described herein, e.g., a composition for inducing a double-strand break within a PCSK9 gene in a cell, for treating a subject having a PCSK9-related disease or condition. In some aspects, the present disclosure provides a pharmaceutical composition comprising a guide RNA as described herein, or a composition as described herein, e.g., a composition for inducing a double-strand break within a PCSK9 gene in a cell, for treating a subject having a PCSK9-related disease or condition.

In some aspects, the present disclosure provides a method of inducing a double-strand break or a single-strand break within a PCSK9 gene in a cell, or reducing expression of a PCSK9 protein in a cell, comprising contacting a cell with a guide RNA as described herein, or a composition as described herein. In some aspects, the present disclosure provides a method of inducing a double-strand break within a PCSK9 gene in a cell, or reducing expression of a PCSK9 protein in a cell, comprising contacting a cell with a guide RNA as described herein, or a composition as described herein. In some embodiments, the cell is in a subject. In some embodiments, the level of PCSK9 protein is measured in a subject sample selected from blood or serum.

In some aspects, the present disclosure provides use of a guide RNA as described herein, or a composition as described herein, in the preparation of a medicament for practicing any of the methods as described herein, e.g., for inducing a double-strand break within a PCSK9 gene in a cell.

In some aspects, the present disclosure provides kits comprising the compositions as described herein.

The following is a non-exhaustive listing of embodiments provided herein.

Embodiment 1 is a guide RNA comprising:
A. a targeting sequence comprising a sequence at least 95%, 90%, 85%, or 80% identical to or complementary to the nucleotide sequence of SEQ ID NOs: 9, 1, 2, 7, 13-15, 17, 18, or 20;
B. a targeting sequence comprising a sequence identical to or complementary to at least 17, 18, 19, or 20 contiguous nucleotides of the nucleotide sequence of SEQ ID NOs: 9, 1, 2, 7, 13-15, 17, 18, or 20; or
C. a targeting sequence comprising a targeting sequence identical to the nucleotide sequence of SEQ ID NOs: 9, 1, 2, 7, 13-15, 17, 18, or 20.

Embodiment 2 is the guide of Embodiment 1, comprising a sequence a targeting sequence identical to the nucleotide sequence of SEQ ID NOs: 9, 14, or 18.

Embodiment 3 is the guide RNA of Embodiment 1 or 2, further comprising one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1; or
      b. one or more of positions H1-6 through H1-10 is substituted relative to Exemplary SpyCas9 sgRNA-1; or
   3. the shortened hairpin 1 region lacks 5-10 nucleotides, preferably 5-6 nucleotides, and one or more of positions N18, H1-12, or n is substituted relative to Exemplary SpyCas9 sgRNA-1; or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1; or
C. a substitution relative to Exemplary SpyCas9 sgRNA—at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 with an upper stem region, wherein the upper stem modification comprises a modification to any one or more of US1-US12 in the upper stem region.

Embodiment 4 is the guide RNA of Embodiment 3, wherein the guide RNA lacks 6 nucleotides in shortened hairpin 1.

Embodiment 5 is the guide RNA of Embodiment 3, wherein the guide RNA lacks 8 nucleotides in shortened hairpin 1.

Embodiment 6 is the guide RNA of any one of Embodiments 3-5, wherein H-1 and H-3 are deleted.

Embodiment 7 is the guide RNA of any one of Embodiments 3-6, wherein the guide RNA further comprises a 3′ tail.

Embodiment 8 is the guide RNA of Embodiment 7, wherein the 3′ tail is 1˜4 nucleotides in length, optionally 1 nucleotide in length.

Embodiment 9 is the guide RNA of any one of Embodiments 3-8, wherein the guide RNA comprises an upper stem region comprising a modification to any one or more of US1-US12 in the upper stem region.

Embodiment 10 is the guide RNA of Embodiment 1 or 2, comprising a modified nucleotide sequence according to the pattern (mN*)3(N)13-17, wherein “m” is indicative of a 2′-O-methyl modification, * is indicative of a phosphorothioate bond, and N is indicative of a 2′-OH and a phosphodiester bond.

Embodiment 11 is the guide RNA of Embodiment 1, wherein the guide RNA comprises a modified nucleotide sequence selected from a sequence in Table 4A (SEQ ID NO: 501-512, optionally SEQ ID NO: 507 or 512), wherein the modified nucleotide sequence is 3′ of the guide sequence.

Embodiment 12 is the guide RNA of Embodiment 11, modified according to the pattern of nucleotide sequence selected from a sequence in Table 4B (SEQ ID NO: 601-612, optionally SEQ ID NO: 607 or 612), wherein the (mN*)3N17 refers to the targeting sequence of Embodiment 1 or 2.

Embodiment 13 is the guide RNA of any one of Embodiments 1-12, wherein the guide RNA comprises the nucleotide sequence selected from SEQ ID NOs: 121, 109, 101, 102, 107, 113-115, 117, 118, 120, 122, or 123, optionally SEQ ID NOs: 109, 114, 118, 121, 122, or 123 as provided in Table 2.

Embodiment 14 is the guide RNA of Embodiment 13, wherein each nucleotide is any natural or non-natural nucleotide.

Embodiment 15 is the guide RNA of Embodiment 14, wherein the guide RNA comprises the modified nucleotide sequence selected from SEQ ID Nos: 221, 209, 201, 202, 207, 213-215, 217, 218, 220, 222, or 223, optionally SEQ ID NOs: 209, 214, 218, 221, 222, or 223 as provided in Table 2.

Embodiment 16 is a composition comprising a guide RNA of any one of Embodiments 1-15.

Embodiment 17 is the composition of Embodiment 16, further comprising an RNA-guided DNA binding agent or a nucleic acid encoding an RNA-guided DNA binding agent.

Embodiment 18 is the composition of Embodiment 17, wherein the nucleic acid encoding the RNA-guided DNA binding agent comprises an mRNA comprising an open reading frame (ORF) encoding the RNA-guided DNA binding agent.

Embodiment 19 is the composition of Embodiment 17 or 18, wherein the RNA-guided DNA binding agent is a Cas9 nuclease.

Embodiment 20 is the composition of Embodiment 19, wherein the Cas9 is S. pyogenes Cas9.

Embodiment 21 is the composition of Embodiment 20, wherein the S. pyogenes Cas9 comprises an amino acid sequence having at least 90% identity to SEQ ID NOs: 1001, 1004, 1007, or 1010, or an ORF encoding a S. pyogenes Cas9 having at least 90% identity to a sequence selected from SEQ ID NOs: 1003, 1006, and 1009.

Embodiment 22 is the composition of Embodiment 21, wherein the ORF encoding the amino acid sequence has at least 95% identity to SEQ ID NOs: 1003, 1006, or 1009.

Embodiment 23 is the composition of any one of Embodiments 19-22, wherein the nuclease has double-stranded endonuclease activity.

Embodiment 24 is the composition of any one of Embodiments 18-23, wherein the ORF is a modified ORF.

Embodiment 25 is the composition of Embodiment 21, wherein the guide RNA comprises a targeting sequence identical to the nucleotide sequence of SEQ ID NO: 9 and the S. pyogenes Cas9 comprises an amino acid sequence having at least 95% identity to SEQ ID NOs: 1001, wherein the S. pyogenes Cas9 wherein the nuclease has double stranded endonuclease activity.

Embodiment 26 is the composition of Embodiment 21, wherein the guide RNA comprises a targeting sequence comprising a sequence identical to the nucleotide sequence of SEQ ID NO: 9 and the S. pyogenes Cas9 comprises an amino acid sequence comprising the amino acid sequence of SEQ ID NOs: 1001.

Embodiment 27 is the composition of Embodiment 21, wherein the guide RNA comprises a targeting sequence comprising a sequence identical to the nucleotide sequence of SEQ ID NO: 9 and wherein the S. pyogenes Cas9 an ORF encoding a S. pyogenes Cas9 having at least 90% identity to a sequence selected from SEQ ID NOs: 1003, wherein the S. pyogenes Cas9 wherein the nuclease has double stranded endonuclease activity.

Embodiment 28 is the composition of any one of Embodiments 25-27, wherein the ORF is a modified ORF.

Embodiment 29 is the composition of any one of Embodiments 25-28, wherein the guide RNA comprises the nucleotide sequence of SEQ ID NO: 121 or 109.

Embodiment 30 is the composition of any one of Embodiments 25-28, wherein the guide RNA comprises the modified nucleotide sequence of SEQ ID NO: 221 or 209.

Embodiment 31 is the composition of any one of Embodiments 16-30, further comprising a pharmaceutical excipient.

Embodiment 32 is the composition of any one of Embodiments 16-31, wherein the guide RNA is associated with a lipid nanoparticle (LNP).

Embodiment 33 is the composition of Embodiment 32, wherein the LNP comprises a cationic lipid.

Embodiment 34 is the composition of Embodiment 33, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 35 is the composition of any one of Embodiments 32-34, wherein the LNP comprises a helper lipid.

Embodiment 36 is the composition of Embodiment 35, wherein the helper lipid is cholesterol.

Embodiment 37 is the composition of any one of Embodiments 32-36, wherein the LNP comprises a neutral lipid.

Embodiment 38 is the composition of Embodiment 37, wherein the neutral lipid is 1,2-distearoyl-sn-glycero-3-phosphocholine (DSPC).

Embodiment 39 is the composition of any one of Embodiments 32-38, wherein the LNP comprises a stealth lipid.

Embodiment 40 is the composition of Embodiment 39, wherein the stealth lipid is 1,2-dimyristoyl-rac-glycero-3-methoxypolyethylene glycol-2000 (PEG2k-DMG).

Embodiment 41 is the composition of Embodiment 32, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate, DSPC, cholesterol, and PEG2k-DMG.

Embodiment 42 is a pharmaceutical composition comprising the guide RNA of any one of Embodiments 1-15 or the composition of any one of Embodiments 16-41.

Embodiment 43 is a pharmaceutical composition comprising, or use of, the guide RNA of any one of Embodiments 1-15 or the composition of any one of Embodiments 16-41 for inducing a double-strand break or a single-strand break within a PCSK9 gene in a cell or reducing expression of a PCSK9 gene in a cell.

Embodiment 44 is the pharmaceutical composition or use of Embodiment 43, wherein the cell is a liver cell.

Embodiment 45 is the pharmaceutical composition or use of Embodiment 44, wherein the cell is in a subject.

Embodiment 46 is a pharmaceutical composition comprising, or use of, the guide RNA of any one of Embodiments 1-15 or the composition of any one of Embodiments 16-41 for treating a subject having a PCSK9 related disease.

Embodiment 47 is a method of inducing a double-strand break or a single-strand break within a PCSK9 gene in a cell or reducing expression of a PCSK9 protein in a cell comprising contacting a cell with the guide RNA of any one of Embodiments 1-15 and an RNA-guided DNA binding agent or a nucleic acid encoding an RNA-guided DNA binding agent, or the composition of any one of Embodiments 16-41.

Embodiment 48 is the use of the guide RNA of any one of Embodiments 1-15 or the composition of any one of Embodiments 16-41 in the preparation of a medicament for practicing the method of Embodiment 47.

Embodiment 49 is a human liver cell comprising an indel in a nucleotide sequence selected from a genomic locus in Table 1.

Embodiment 50 is the human liver cell of Embodiment 49, comprising an indel in a nucleotide sequence selected from a genomic locus selected from the genomic locus of SEQ ID NO: 9, 1, 2, 7, 13-15, 17, 18, or 20.

Embodiment 51 is a method of modifying a genomic locus in a human liver cell, the method comprising contacting a human liver cell with the guide RNA of any one of Embodiments 1-15 and an RNA-guided DNA binding agent or a nucleic acid encoding an RNA-guided DNA binding agent, or the composition of any one of Embodiments 16-41.

Embodiment 52 is the method of Embodiment 51, wherein the method is performed in vivo.

Embodiment 53 is the pharmaceutical composition, method, or cell of any one of Embodiments 44, 45, 49-52, wherein the liver cell is a hepatocyte.

Embodiment 54 is the pharmaceutical composition, method, or cell of Embodiment 53, wherein the cell is in a subject with a PCSK9 related disease.

Embodiment 55 is a method of treating a PCSK9 related disease in a subject, the method comprising administering to the subject the guide RNA of any one of Embodiments 1-15 and an RNA-guided DNA binding agent or a nucleic acid encoding an RNA-guided DNA binding agent, or the composition of any one of Embodiments 16-41, or the pharmaceutical composition of Embodiment 42.

Embodiment 56 is the pharmaceutical composition, method, or cell of any one of Embodiments 42-55, further comprising determining the PCSK9 protein level in a subject blood or serum sample.

Embodiment 57 is the use of the guide RNA of any one of Embodiments 1-15 or the composition of any one of Embodiments 16-41, or the pharmaceutical composition of Embodiment 42 in the preparation of a medicament for practicing any of the methods of Embodiments 47 or 51-56.

Embodiment 58 is a kit comprising the guide RNA of any one of Embodiments 1-15 and an RNA-guided DNA binding agent or a nucleic acid encoding an RNA-guided DNA binding agent, the composition of any one of Embodiments 16-41, or the pharmaceutical composition of any one of Embodiments 42-46.

Embodiment 59 is a kit for use or for practicing the method of any one of Embodiments 47 or 51-56.

### FIGURE LEGENDS

FIG. 1 shows dose dependent curves of mean percent editing at the PCSK9 locus in primary human hepatocytes (PHH) treated with various sgRNAs and Cas9 mRNA.

FIG. 2 shows dose dependent curves of secreted PCSK9 serum levels for PHH treated with Cas9 mRNA and various sgRNA targeting the PCSK9 locus.

FIG. 3A shows mean percent editing at the inserted human PCSK9 locus in mouse liver following treatment with Cas9 mRNA and the indicated sgRNAs.

FIG. 3B shows percent knockdown (KD) of human PCSK9 serum levels in mice treated with Cas9 mRNA and the indicated sgRNAs.

FIG. 4A shows the mean percent editing at the inserted human PCSK9 locus in mouse liver following treatment with Cas9 mRNA and the indicated sgRNAs.

FIG. 4B shows the human PCSK9 serum levels in mice treated with Cas9 mRNA and the indicated sgRNAs.

FIG. 4C shows percent knockdown (KD) of human PCSK9 serum levels in mice treated with Cas9 mRNA and the indicated sgRNAs.

FIGS. 5A-5B show dose dependent curves of mean percent editing at the PCSK9 locus in primary cynomolgus hepatocytes (PCH) treated with Cas9 mRNA and the indicated sgRNAs.

FIGS. 6A-6C show dose dependent curves of mean percent editing at the PCSK9 locus in PHH treated with Cas9 mRNA and the indicated sgRNAs.

FIGS. 7A-7C show DRC of mean percent editing at the PCSK9 locus in PHH treated with Cas9 mRNA and the indicated sgRNAs.

FIG. 8A shows mean percent editing at the inserted human PCSK9 locus in mouse liver following treatment with Cas9 mRNA and the indicated sgRNAs.

FIG. 8B shows human PCSK9 serum levels in mice treated with Cas9 mRNA and the indicated sgRNAs.

FIG. 8C shows percent knockdown (KD) of human PCSK9 serum levels in mice treated with Cas9 mRNA and the indicated sgRNAs.

BRIEF DESCRIPTION OF DISCLOSED SEQUENCES

| SEQ ID NO | Description |
|---|---|
| 1-20 | Exemplary PCSK9 guide sequences and chromosomal |
| | coordinates |
| 101-123 | Exemplary unmodified sgRNA sequences targeting PCSK9 |
| 201-223 | Exemplary modified sgRNA sequences targeting PCSK9 |
| 301-309 | Exemplary unmodified SpyCas9 scaffold sequences |
| 401-408 | Exemplary unmodified SpyCas9 guide RNA sequences |
| 501-512 | Exemplary modified SpyCas9 guide scaffold sequences |
| 601-612 | Exemplary modified SpyCas9 guide sequences |
| 1001 | Cas9 amino acid sequence with 1x NLS (RNP) |
| 1002 | mRNA encoding Spy Cas9 |
| 1003 | Open reading frame for Spy Cas9 |
| 1004 | Amino acid sequence for Spy Cas9 |
| 1005 | mRNA encoding Spy Cas9 |
| 1006 | Open reading frame for Spy Cas9 |
| 1007 | Amino acid sequence for Spy Cas9 |
| 1008 | mRNA encoding Spy Cas9 with HiBiT tag |
| 1009 | Open reading frame for Spy Cas9 with HiBiT tag |
| 1010 | Amino acid sequence for SpyCas9 with HiBiT tag |
| 1011 | Exemplary EMX1 guide RNA (G000644) |
| 1012 | Exemplary VEGFA guide RNA (G000645) |
| 1013 | Exemplary SV40 NLS |
| 1014 | Exemplary SV40 NLS |
| 1015 | Exemplary nucleoplasmin NLS |

### DETAILED DESCRIPTION

Reference will now be made in detail to certain embodiments of the disclosure, examples of which are illustrated in the accompanying drawings. While the present teachings are described in conjunction with various embodiments, it is not intended to limit the present teachings to those embodiments. On the contrary, the present teachings encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended claims, the singular form “a”, “an”, and “the” include plural references unless the context clearly dictates otherwise. Thus, for example, reference to “a conjugate” includes a plurality of conjugates and reference to “a cell” includes a plurality of cells (e.g., a population of cells) and the like.

Numeric ranges are inclusive of the numbers defining the range. Measured and measurable values are understood to be approximate, taking into account significant digits and the error associated with the measurement.

The use of “comprise”, “comprises”, “comprising”, “contain”, “contains”, “containing”, “include”, “includes”, and “including” is not intended to be limiting. It is to be understood that both the foregoing general description and detailed description are exemplary and explanatory only and are not restrictive of the teachings. Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense in the specification, i.e., equivalent to “and/or,” unless the context clearly indicates otherwise.

The term “about”, when used before a list, modifies each member of the list. The term “about” is understood to encompass tolerated variation or error within the art, e.g., 2 standard deviations from the mean, or the sensitivity of the method used to take a measurement. When “about” is present before the first value of a series, it can be understood to modify each value in the series.

Ranges are understood to include the numbers at the end of the range and all logical values therebetween. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

In the event of a conflict between a sequence in the application and an indicated accession number or position in an accession number, the sequence in the application predominates.

As used herein, “detecting an analyte” and the like is understood as performing an assay in which the analyte can be detected, if present, wherein the analyte is present in an amount above the level of detection of the assay.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

The section headings used herein are for organizational purposes only and are not to be construed as limiting the desired subject matter in any way. In the event that any material incorporated by reference contradicts any term defined in this specification or any other express content of this specification, this specification controls.

### I. DEFINITIONS

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy, 2′ halide, or 2′-O-(2-methoxyethyl) (2′-O-moe) substitutions. An RNA may comprise one or more deoxyribose nucleotides, e.g. as modifications, and similarly a DNA may comprise one or more ribonucleotides. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, 06-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analog containing one or more LNA nucleotide monomers with a bicyclic furanose unit locked in an RNA mimicking sugar conformation, which enhances hybridization affinity toward complementary RNA and DNA sequences (Vester and Wengel, 2004, Biochemistry 43(42):13233-41). Nucleic acid includes “unlocked nucleic acid” which enables the modulation of the thermodynamic stability and also provide nuclease stability. RNA and DNA have different sugar moieties and can differ by the presence of uridine or analogs thereof in RNA and thymine or analogs thereof in DNA.

“Polypeptide” as used herein refers to a multimeric compound comprising amino acid residues that can adopt a three-dimensional conformation. Polypeptides include but are not limited to enzymes, enzyme precursor proteins, regulatory proteins, structural proteins, receptors, nucleic acid binding proteins, antibodies, etc. Polypeptides may, but do not necessarily, comprise post-translational modifications, non-natural amino acids, prosthetic groups, and the like.

“Guide RNA”, “gRNA”, and simply “guide” are used herein interchangeably to refer to, for example, either a single guide RNA or the combination of a crRNA and a trRNA (also known as tracrRNA). The crRNA and trRNA may be associated as a single RNA strand (as a single guide RNA, sgRNA) or, for example, in two separate RNA strands (dual guide RNA, dgRNA). “Guide RNA” or “gRNA” refers to either a sgRNA or a dgRNA. The trRNA may be a naturally-occurring sequence, or may comprise modifications or variations. Such modifications or variations may be chemically induced.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be about 20 nucleotides in length, for example when used in combination with an RNA-guided DNA binding agent such as Streptococcus pyogenes (i.e., “Spy”) Cas9. Preferred guide sequence lengths for related Cas9 homologs/orthologs, including shorter or longer sequences can also be used and are known in the art.

For example, Spy Cas9 guides can be 16, 17, preferably 18, 19, or 20 nucleotides in length such that, in some embodiments, the Spy Cas9 guide sequence comprises 16, 17, 18, 19, or 20 contiguous nucleotides of a sequence selected from SEQ ID NOs: 1-20, optionally SEQ ID NOs: 1, 2, 7, 9, 13-15, 17, 18, or 20, optionally SEQ ID NOs: 9, 14, or 18. In some embodiments, the target sequence is in a gene or on a chromosome, for example, and is complementary to the guide sequence. In some embodiments, the degree of complementarity or identity between a guide sequence and its corresponding target sequence is at least 80%, 85%, preferably 90%, or 95%, or is 100%. For example, in some embodiments, the guide sequence comprises a sequence of at least 16, 17, preferably 18, 19, or 20 contiguous nucleotides of a sequence selected from SEQ ID NOs: 1, 2, 7, 9, 13-15, 17, 18, or 20, optionally SEQ ID NOs: 9, 14, or 18. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1, 2, 3, or 4 mismatches within the duplex formed by the guide and the target sequence, where the total length of the target sequence is 16, 17, 18, 19, 20 nucleotides, or more. In some embodiments, the guide sequence and the target region may contain 1, 2, 3 or 4 mismatches where the guide sequence comprises at least 20 nucleotides. In some embodiments, the guide sequence and the target region may contain 1, 2, 3, or 4 mismatches where the guide sequence comprises 20 nucleotides. That is, the guide sequence and the target region may form a duplex region having 16, 17, 18, 19, 20 base pairs, or more. In certain embodiments, the duplex region may include 1, 2, 3, or 4 mismatches such that guide strand and target sequence are not fully complementary. For example, a guide strand and target sequence may be complementary over a 20 nucleotide region, including 2 mismatches, such that the guide sequence and target sequence are 90% complementary providing a duplex region of 18 base pairs out of 20. Tolerated mismatch positions are known in the art. For example, protospacer adjacent motif (PAM)-distal mismatches tend to be better tolerated than PAM-proximal matches, and mismatch tolerances at other positions have been characterized (see, e.g., Sternberg et al., 2015, Nature: 527:110-113).

Target sequences for RNA-guided DNA binding agents, as defined by the targeting sequence of a guide RNA, may be present on either the positive or negative strand. Tables and other disclosures provided herein may recite genomic coordinates as a target sequence. It is understood that the guide can be complementary to either the positive or negative strand of the DNA as defined by the genomic coordinates. The sequence to which the guide is complementary depends on the presence of an appropriate PAM for the RNA guided DNA binding agent on the opposite strand. Thus, in some embodiments, where the guide sequence binds the reverse complement of a target sequence, i.e., the guide sequence is identical to certain nucleotides of the sense (positive) strand of the target sequence, when the PAM is present in the sense strand, when the PAM is present in the sense strand, except for the substitution of U for T in the guide sequence.

As used herein, an “RNA-guided DNA binding agent” or “RNA-guided DNA binding protein” means a polypeptide or complex of polypeptides having RNA and DNA binding activity, or a DNA-binding subunit of such a complex, wherein the DNA binding activity is sequence-specific and depends on the presence of a PAM and the sequence of the guide RNA. Exemplary RNA-guided DNA binding agents include Cas cleavases/nickases and inactivated forms thereof (e.g., “dCas DNA binding agents”). “Cas nuclease”, as used herein, encompasses Cas cleavases, Cas nickases, and dCas DNA binding agents. Cas nickases include nucleases in which one of the RuvC or HNH domain of the Cas protein is mutated, such that only a single strand is cleaved by the nuclease. The dCas DNA binding agent may be a dead nuclease comprising non-functional nuclease domains (i.e., a RuvC or HNH domain). In some embodiments the Cas cleavase or Cas nickase encompasses a dCas DNA binding agent modified to permit DNA cleavage, e.g., via fusion with a FokI domain.

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, or nucleic acid sequences encoding the amino acid sequences provided herein are also contemplated. In certain embodiments, the nucleotide sequence encoding the Cas9 amino acid sequence is not a naturally occurring Cas9 nucleotide sequence. Sequences with at least 95%, 96%, 97%, 98%, or 99% identity to any of the Cas9 amino acid sequences provided herein are also contemplated. In certain embodiments, the Cas9 amino acid sequence is not a naturally occurring Cas9 sequence.

Exemplary open reading frames for Cas9 are provided in Table 23 below.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein). Exemplary peptide linkers are disclosed elsewhere herein.

“Modified uridine” is used herein to refer to a nucleoside other than thymidine with the same hydrogen bond acceptors as uridine and one or more structural differences from uridine. In some embodiments, a modified uridine is a substituted uridine, i.e., a uridine in which one or more non-proton substituents (e.g., alkoxy, such as methoxy) takes the place of a proton. In some embodiments, a modified uridine is pseudouridine. In some embodiments, a modified uridine is a substituted pseudouridine, i.e., a pseudouridine in which one or more non-proton substituents (e.g., alkyl, such as methyl) takes the place of a proton. In some embodiments, a modified uridine is any of a substituted uridine, pseudouridine, or a substituted pseudouridine, e.g., N1-methyl-psuedouridine.

“Uridine position” as used herein refers to a position in a polynucleotide occupied by a uridine or a modified uridine. Thus, for example, a polynucleotide in which “100% of the uridine positions are modified uridines” contains a modified uridine at every position that would be a uridine in a conventional RNA (where all bases are standard A, U, C, or G bases) of the same sequence. Unless otherwise indicated, a U in a polynucleotide sequence of a sequence table or sequence listing in or accompanying this disclosure can be a uridine or a modified uridine.

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with the target sequence and the agent binds to the target sequence; in cases where the agent is a cleavase or nickase, binding can be followed by cleaving or nicking.

As used herein, a “control” is understood as an appropriate matched sample or subject for comparison. For example, a control can be a cell population treated in the same manner as the test population except that the treatment used for the control population lacks at least one active agent, e.g., a guide RNA, an mRNA encoding a nuclease, an insertion construct, or a lipid formulation. In certain embodiments, a control may be an internal control, e.g., a cell population or subject prior to treatment.

In certain embodiments, a “control” as in a control subject is a comparator for a measurement, e.g., a diagnostic measurement of a sign or symptom of a disease. In certain embodiments, a control can be a subject sample from the same subject at an earlier time point, e.g., before a treatment intervention. In certain embodiments, a control can be a measurement from a normal subject, i.e., a subject not having the disease of the treated subject, to provide a normal control, e.g., an enzyme concentration or activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. The ability to design or select appropriate controls is within the ability of those of skill in the art. It is understood when relative values are provided, they can be considered as relative values as compared to an appropriate control.

As used herein, “purified” such as in “purified composition,” “purified protein,” or “purified nucleic acid,” and the like, refers to a composition (or the like, e.g., protein or nucleic acid) where at least some non-composition (or the like) components have been removed by human intervention from an initial composition or the mixture in which it was made, e.g., a cell, a subject sample, or a reaction mixture. In certain embodiments, when the term “purified” is used, the composition (or the like, e.g., protein or nucleic acid) is the major component, such as comprising at least 80%, 85%, 90%, or 95% free of other components.

As used herein, “subject” includes primates, including human and non-human primates, mouse, and rat. In certain embodiments, the subject is a human subject. In certain embodiments, the subject is a non-human subject. In certain embodiments, the subject is a non-human subject expressing one or more human genes, e.g., a transgenic mouse expressing a human gene, or a mouse in which the liver has been repopulated with human hepatocytes. Such models are well known in the art.

As used herein, a “target sequence” refers to a sequence of nucleic acid in a target gene, in either the positive or the negative strand, that has complementarity to the guide sequence of the gRNA, i.e., that is sufficiently complementary to the guide sequence to permit specific binding of the guide sequence. The interaction of the target sequence and the guide sequence directs an RNA-guided DNA binding agent to bind, and potentially nick or cleave (depending on the activity of the agent), within the target sequence. The specific length of the target sequence and the number of mismatches possible between the target sequence and the guide sequence depend, for example, on the identity of the Cas nuclease being directed by the gRNA.

As used herein, a first sequence is considered to be “identical” or have “100% identity” with a second sequence if an alignment of the first sequence to the second sequence shows that all of the positions of the second sequence in its entirety are matched by the first sequence. For example, the sequence AAG has 100% identity to the sequence AAGA because an alignment would give 100% identity in that there are matches, without gaps, to all three positions of the first sequence. Less than 100% identity can be calculated using routine methods. For example, ACG would have 67% identity with AAGA as two of the three positions of the first sequence are matches to the second sequence (⅔=67%). The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate.

Similarly, as used herein, a first sequence is considered to be “fully complementary” or “100% complementary” to a second sequence when all of the nucleotides of a first sequence are complementary to a second sequence, without gaps. For example, the sequence UCU would be considered to be fully complementary to the sequence AAGA as each of the nucleobases from the first sequence basepair with the nucleotides of the second sequence, without gaps. The sequence UGU would be considered to be 67% complementary to the sequence AAGA as two of the three nucleobases of the first sequence basepair with nucleobases of the second sequence. One skilled in the art will understand that algorithms are available with various parameter settings to determine percent complementarity for any pair of sequences using, e.g., the NCBI BLAST interface (blast.ncbi.nlm.nih.gov/Blast.cgi) or the Needleman-Wunsch algorithm.

“Messenger RNA” or “mRNA” is used herein to refer to a polynucleotide that comprises an open reading frame that can be translated into a polypeptide (i.e., can serve as a substrate for translation by a ribosome and amino-acylated tRNAs). mRNA can comprise one or more chemically modified nucleosides such as 5-methyl-cytidine (5mC), 2-thio-uridine (2sU), N1-methylpseudouridine (m1 ψU) and pseudo-uridine (ψU), or a modified cap structure as provided below.

Exemplary guide sequences useful in the guide RNA compositions and methods described herein are shown in Table 1 and throughout the application. For example, where Table 1 shows a guide sequence, this guide sequence may be used in a guide RNA to direct a RNA-guided DNA binding agent, e.g., a nuclease, such as a Cas nuclease, such as Cas9, to a target sequence. Target sequences are provided in Table 1 as genomic coordinates, and include both the positive and negative strands of genomic DNA (i.e., the sequence given and the sequence's reverse complement). In some embodiments, where the guide sequence binds the reverse complement of a target sequence, the guide sequence is identical to certain nucleotides of the target sequence, except for the substitution of U for T in the guide sequence.

As used herein, “indels” refer to insertion/deletion mutations consisting of a number of nucleotides that are either inserted or deleted at the site of double-strand breaks (DSBs) in a target nucleic acid. As used herein, when indel formation results in an insertion, the insertion is a random insertion at the site of a double-strand break and is not directed by or based on a template sequence.

As used herein, “inhibit expression” and the like refer to a decrease in expression (e.g., knockdown or knockout) of a particular gene product (e.g., protein, mRNA, or both). Expression of a protein (i.e., gene product) can be measured by detecting total cellular amount of the protein from a tissue sample, e.g., biopsy, or cell population of interest by detecting expression of a protein in individual members of a population of cells, e.g., by cell sorting to define percent of cells expressing a protein, or expression of a protein in cells in aggregate, e.g., by ELISA or western blot. Inhibition of expression can result from genetic modification of a gene sequence, e.g., a genomic sequence, such that the full-length gene product, or any gene product, is no longer detected, e.g., knockdown of the gene. Certain genetic modifications can result in the introduction of frameshift or nonsense mutations that prevent translation of the full-length gene product. Genetic modifications at a splice site, e.g., at a position sufficiently close to a splice acceptor site or a splice donor site to disrupt splicing, can prevent translation of the full-length protein. Inhibition of expression can result from a genetic modification in a regulatory sequence within the genomic sequence required for the expression of the gene product, e.g., a promoter sequence, a 3′ UTR sequence, e.g., a capping sequence, a 5′ UTR sequence, e.g., a poly A sequence. Inhibition of expression may also result from disrupting expression or activity of regulatory factors required for translation of the gene product, e.g., production of no gene product. For example, a genetic modification in a transcription factor sequence, inhibiting expression of the full-length transcription factor, can have downstream effects and inhibit expression of one or more gene products controlled by the transcription factor. Inhibition of expression can be predicted by changes in genomic or mRNA sequences. Mutations expected to result in inhibition of expression can be detected by known methods including next generation sequencing of DNA isolated from a tissue sample or cell population of interest. Inhibition of expression can be determined as the percent of cells in a population having a predetermined level of expression of a protein, i.e., a reduction of the percent or number of cells in a population expressing a protein of interest at least a certain level. Inhibition of expression can also be assessed by determining a decrease in overall protein level, e.g., in a cell or tissue sample, e.g., a biopsy sample. In certain embodiments, inhibition of expression of a secreted protein can be assessed in a fluid sample, e.g., cell culture media or a body fluid. Proteins may be present in a body fluid, e.g., blood or urine, to permit analysis of protein level. In certain embodiments, protein level may be determined by protein activity or the level of a metabolic product, e.g., in urine or blood. In some embodiments, “inhibition of expression” may refer to some loss of expression of a particular gene product, for example a decrease in the amount of an mRNA or a protein expressed in a tissue sample or by a population of cells. In some embodiments, “inhibition” may refer to some loss of expression of a particular gene product, for example at the cell surface or secreted into a bodily fluid, e.g., blood. In some embodiments, “inhibition” may refer to some loss of expression in one, or more, cell or tissue types, but not all cell or tissue types, e.g., inhibition of expression in liver, but not in other organs. It is understood that the level of inhibition of expression is relative to a starting level, a reference level, or a control level, in the same type of subject sample. For example, routine monitoring of a protein level may be performed in a fluid sample from a subject, e.g., blood or urine, or in a tissue sample, e.g., a biopsy sample. In certain embodiments, a correlation is known, or established, wherein the level of a biomarker, e.g., in blood or urine, is correlated with the level of inhibition of expression of a target gene. It is understood that the level of inhibition of expression is for the sample being assayed. Similarly, in animal studies where serial tissue samples may be obtained, e.g., liver tissue, the target may be expressed in other tissues. Therefore, the level of inhibition of expression is not necessarily the level of inhibition of expression systemically, but within the tissue, cell type, or fluid being sampled.

As used herein, a “genetic modification” is a change at the DNA level, e.g., induced by a CRISPR/Cas9 gRNA and Cas9 system. A genetic modification may comprise an insertion, deletion, or substitution (i.e., base sequence substitution, i.e., mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g., contiguous nucleotides. A genetic modification can be in a coding sequence, e.g., an exon sequence. A genetic modification can be at a splice site, i.e., sufficiently close to a splice acceptor site or a splice donor site to disrupt splicing. A genetic modification can include insertion of a nucleotide sequence not endogenous to the genomic locus, e.g., insertion of a coding sequence of a heterologous open reading frame or gene. As used herein, a genetic modification can be used to prevent translation of an endogenous full-length protein having an amino acid sequence of the full-length protein prior to genetic modification of the genomic locus. Prevention of translation of a full-length protein or gene product includes prevention of translation of a protein or gene product of any length. Translation of an endogenous full-length protein can be prevented, for example, by a frameshift mutation that results in the generation of a premature stop codon or by generation of a nonsense mutation. Translation of an endogenous full-length protein can be prevented by disruption of splicing. Translation of a full-length protein can be prevented by the insertion of a heterologous coding sequence. Translation of an endogenous full-length protein, e.g., when the endogenous full-length protein contains an unwanted mutation, can be prevented by making a change at one or more positions to change an endogenous full-length protein coding sequence to provide a modified full-length coding sequence different from the endogenous sequence present in the cell, e.g., correction of a point mutation. Translation of an endogenous full-length protein can be prevented by altering the splicing of the endogenous full-length protein to produce a different protein by alternative splicing.

“Treatment” as used herein is understood as reducing at least one sign or symptom of the disease or indication. Reduction can include to a frequency or severity such that the sign or symptom of the disease is no longer detectable. Treatment can include administration of more than one dose of the agent. Treatment can include administration with other agents. Effective treatment does not require a cure or complete elimination of the disease or indication. The rate of progression or development of a disease can be compared to the progression or development of a disease in an appropriately matched control, e.g., a population control, a control from a natural history study. As used herein, “delivering” and “administering” are used interchangeably.

Co-administration, as used herein, means that a plurality of substances are administered sufficiently close together in time so that the agents act together. Co-administration encompasses administering substances together in a single formulation and administering substances in separate formulations close enough in time so that the agents act together.

As used herein, the phrase “pharmaceutically acceptable” means that which is useful in preparing a pharmaceutical composition that is generally non-toxic and is not biologically undesirable and that are not otherwise unacceptable for pharmaceutical use. Pharmaceutically acceptable generally refers to substances that are non-pyrogenic. Pharmaceutically acceptable can refer to substances that are sterile, especially for pharmaceutical substances that are for injection or infusion.

As used herein, “PCSK9” refers to the nucleic acid sequence or protein sequence of “proprotein convertase subtilisin kexin 9” or “proprotein convertase subtilisin kexin type 9.” The human wild-type PCSK9 sequence is available at NCBI Gene ID: 255738 (worldwide web at ncbi.nlm.nih.gov/gene?cmd-retrieve&dopt=default&rn=1&list_uids=255738, in the version available on the date of filing the instant application); Ensembl: ENSG00000169174 MIM:607786, chr1:55039548-chr1:55064852. Synonyms for PCSK9 include NARC1, FH3, HCHOLA3, PC9, FHCL3 and LDLCQ1. The PCSK9 gene encodes a member of the subtilisin-like proprotein convertase family, which includes proteases that process protein and peptide precursors trafficking through regulated or constitutive branches of the secretory pathway. The encoded protein undergoes an autocatalytic processing event with its prosegment in the ER and is constitutively secreted as an inactive protease into the extracellular matrix and trans-Golgi network. It is expressed in liver, intestine, and kidney tissues and escorts specific receptors for lysosomal degradation. The PCSK9 protease is involved in regulating circulating LDL cholesterol levels, and plays a role in cholesterol and fatty acid metabolism. Certain mutations or excess production of PCSK9 have been associated with cardiovascular disease and chronic liver injury. Single nucleotide polymorphisms and other variations of the human PCSK9 sequence can be found, for example, at www.ncbi.nlm.nih.gov/SNP/snp_ref.cgi?locusId=255738.

As used herein, the term “within the genomic coordinates” includes the boundaries of the genomic coordinate range given. For example, if chr1: 55039548-chr1:55064852 is given, the coordinates chr1: 55039548 and chr1:55064852 are encompassed. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

### II. COMPOSITIONS

Compositions Comprising Guide RNA (gRNAs)

Provided herein are compositions useful for altering a DNA sequence, e.g., inducing a single-strand (SSB) or double-strand break (DSB), within a PCSK9 gene, e.g., using a guide RNA with an RNA-guided DNA binding agent (e.g., a CRISPR/Cas system). Guide sequences targeting a PCSK9 gene are shown in Table 1 at SEQ ID NOs: 1-20, as are the genomic coordinates that such guide RNA targets.

Each of the guide sequences shown in Table 1 at SEQ ID NOs: 1-20 may further comprise additional nucleotides to form a crRNA, e.g., with the following exemplary nucleotide sequence following the guide sequence at its 3′ end: GUUUUAGAGCUAUGCUGUUUUG (SEQ ID NO: 301) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGC (SEQ ID NO: 303) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 302) in 5′ to 3′ orientation.

In the case of a sgRNA, the guide sequences may be integrated into the following modified motif: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 601), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage to the adjacent nucleotide residue; and wherein the N's are collectively the nucleotide sequence of a guide sequence. In the context of a modified sequence, A, C, G, N, and U are an unmodified RNA nucleotide, i.e., a 2′-OH sugar moiety with a phosphodiesterase linkage to the adjacent nucleotide residue, or a 5′-terminal PO₄.

In the case of a sgRNA, the guide sequences may further comprise a SpyCas9 sgRNA sequence. An example of a SpyCas9 sgRNA sequence is shown in the table below (SEQ ID NO: 303: GUUUUAGAGCUAGAAAUAGCAAGUUAAAAUAAGGCUAGUC CGUUAUCAACUUGAAAAAGUGGCACCGAGU CGGUGC-“Exemplary SpyCas9 sgRNA-1”), included at the 3′ end of the guide sequence, and provided with the domains as shown in Table A below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, or an inverted abasic modified nucleotide; or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 303 (“Exemplary SpyCas9 sgRNA-1” as shown in Table A) as an example, the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 303) or
      b. one or more of positions H1-6 through H1-10 is substituted relative to Exemplary SpyCas9 sgRNA-1 (SEQ ID NO: 303); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 303); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 303); or
C. a substitution relative to Exemplary SpyCas9 sgRNA-1 (SEQ ID NO: 303) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 (SEQ ID NO: 303) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-OMe modified nucleotide.

In certain embodiments, the Exemplary SpyCas9 sgRNA-1 lacks 6 nucleotides in shortened hairpin 1.

In certain embodiments, the Exemplary SpyCas9 sgRNA-1 lacks 8 nucleotides in shortened hairpin 1.

In certain embodiments, in the Exemplary SpyCas9 sgRNA-1 H-1 and H-3 are deleted.

In certain embodiments, the Exemplary SpyCas9 sgRNA-1 further comprises a 3′ tail. In certain embodiments, the 3′ tail is 1˜4 nucleotides in length, optionally 1 nucleotide in length.

In certain embodiments, the Exemplary SpyCas9 sgRNA-1 comprises an upper stem region comprising a modification to any one or more of US1-US12 in the upper stem region.

In certain embodiments, Exemplary SpyCas9 sgRNA-1, or an sgRNA, such as an sgRNA comprising an Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a 2′ deoxy (2′H—) modified nucleotide, an abasic nucleotide, a locked nucleic acid (LNA) nucleotide, an unlocked nucleic acid (UNA) nucleotide, or a phosphorothioate (PS) linkage between nucleotides, a terminal inverted abasic modified nucleotide; or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

TABLE A
Exemplary spyCas9 sgRNA-1 (SEQ ID NO: 303)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B3-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 1
PCSK9 guide sequences and chromosomal coordinates

| Guide | | Guide Target | SEQ | Genomic Coordinates |
|---|---|---|---|---|
| ID | Strand | Sequence | ID NO | (hg38) |
| G016649 | + | GGUGCUAGCCUUGCGUU | 1 | chr1: 55039959-55039979 |
| | | CCG | | |
| G016650 | + | GCUAGCCUUGCGUUCCG | 2 | chr1: 55039962-55039982 |
| | | AGG | | |
| G016654 | − | CCCGCACCUUGGCGCAG | 3 | chr1: 55040031-55040051 |
| | | CGG | | |
| G016657 | − | UCUUGGUGAGGUAUCCC | 4 | chr1: 55043946-55043966 |
| | | CGG | | |
| G016660 | + | GAAGAUGAGUGGCGACC | 5 | chr1: 55044006-55044026 |
| | | UGC | | |
| G016661 | − | GUCGACAUGGGGCAACU | 6 | chr1: 55046526-55046546 |
| | | UCA | | |
| G016662 | + | GCCCCAUGUCGACUACA | 7 | chr1: 55046533-55046553 |
| | | UCG | | |
| G016674 | − | CUUGGCAGUUGAGCACG | 8 | chr1: 55052741-55052761 |
| | | CGC | | |
| G016675 | + | CGUGCUCAACUGCCAAG | 9 | chr1: 55052744-55052764 |
| | | GGA | | |
| G016687 | − | CCUCAGCACAGGCGGCU | 10 | chr1: 55061413-55061433 |
| | | UGU | | |
| G016689 | − | CACUGGUUGGGCUGACC | 11 | chr1: 55061436-55061456 |
| | | UCG | | |
| G016690 | − | UCCUGCGCACGGGCGCC | 12 | chr1: 55039912-55039932 |
| | | CGC | | |
| G016696 | + | GUUGCCUGGCACCUACG | 13 | chr1: 55043853-55043873 |
| | | UGG | | |
| G016704 | + | UACCCCUCCACGGUACC | 14 | chr1: 55046605-55046625 |
| | | GGG | | |
| G016707 | − | UCAUCCGCCCGGUACCG | 15 | chr1: 55046612-55046632 |
| | | UGG | | |
| G016709 | + | AUGUCGCCUUGGAAAGA | 16 | chr1: 55052261-55052281 |
| | | CGG | | |
| G016714 | − | GGGCCAUCACUUACCUA | 17 | chr1: 55052785-55052805 |
| | | UGA | | |
| G016723 | + | CAGCACACUCGGGGCCU | 18 | chr1: 55058528-55058548 |
| | | ACA | | |
| G016730 | − | UGUCUACGGCGUAGGCC | 19 | chr1: 55063439-55063459 |
| | | CCC | | |
| G016735 | − | UGCCUGUAGUGCUGACG | 20 | chr1: 55063481-55063501 |
| | | UCC | | |

TABLE 2
Exemplary unmodified and modified sgRNA sequences targeting PCSK9

| Guide | Unmodified | SEQ | | SEQ |
|---|---|---|---|---|
| ID | guide | ID NO | Modified guide | ID NO |
| G016649 | GGUGCUAGCCUU | 101 | mG*mG*mU*GCUAGCCUUGC | 201 |
| | GCGUUCCGGUUU | | GUUCCGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016650 | GCUAGCCUUGCG | 102 | mG*mC*mU*AGCCUUGCGUU | 202 |
| | UUCCGAGGGUUU | | CCGAGGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016654 | CCCGCACCUUGG | 103 | mC*mC*mC*GCACCUUGGCG | 203 |
| | CGCAGCGGGUUU | | CAGCGGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016657 | UCUUGGUGAGGU | 104 | mU*mC*mU*UGGUGAGGUA | 204 |
| | AUCCCCGGGUUU | | UCCCCGGGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016660 | GAAGAUGAGUGG | 105 | mG*mA*mA*GAUGAGUGGC | 205 |
| | CGACCUGCGUUU | | GACCUGCGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016661 | GUCGACAUGGGG | 106 | mG*mU*mC*GACAUGGGGC | 206 |
| | CAACUUCAGUUU | | AACUUCAGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016662 | GCCCCAUGUCGA | 107 | mG*mC*mC*CCAUGUCGACU | 207 |
| | CUACAUCGGUUU | | ACAUCGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016674 | CUUGGCAGUUGA | 108 | mC*mU*mU*GGCAGUUGAG | 208 |
| | GCACGCGCGUUU | | CACGCGCGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016675 | CGUGCUCAACUG | 109 | mC*mG*mU*GCUCAACUGCC | 209 |
| | CCAAGGGAGUUU | | AAGGGAGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016687 | CCUCAGCACAGG | 110 | mC*mC*mU*CAGCACAGGCG | 210 |
| | CGGCUUGUGUUU | | GCUUGUGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016689 | CACUGGUUGGGC | 111 | mC*mA*mC*UGGUUGGGCU | 211 |
| | UGACCUCGGUUU | | GACCUCGGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016690 | UCCUGCGCACGG | 112 | mU*mC*mC*UGCGCACGGGC | 212 |
| | GCGCCCGCGUUU | | GCCCGCGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016696 | GUUGCCUGGCAC | 113 | mG*mU*mU*GCCUGGCACCU | 213 |
| | CUACGUGGGUUU | | ACGUGGGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016704 | UACCCCUCCACG | 114 | mU*mA*mC*CCCUCCACGGU | 214 |
| | GUACCGGGGUUU | | ACCGGGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016707 | UCAUCCGCCCGG | 115 | mU*mC*mA*UCCGCCCGGUA | 215 |
| | UACCGUGGGUUU | | CCGUGGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016709 | AUGUCGCCUUGG | 116 | mA*mU*mG*UCGCCUUGGA | 216 |
| | AAAGACGGGUUU | | AAGACGGGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016714 | GGGCCAUCACUU | 117 | mG*mG*mG*CCAUCACUUAC | 217 |
| | ACCUAUGAGUUU | | CUAUGAGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016723 | CAGCACACUCGG | 118 | mC*mA*mG*CACACUCGGGG | 218 |
| | GGCCUACAGUUU | | CCUACAGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016730 | UGUCUACGGCGU | 119 | mU*mG*mU*CUACGGCGUA | 219 |
| | AGGCCCCCGUUU | | GGCCCCCGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G016735 | UGCCUGUAGUGC | 120 | mU*mG*mC*CUGUAGUGCU | 220 |
| | UGACGUCCGUUU | | GACGUCCGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCAmAmCmUmU | |
| | CGUUAUCAACUU | | mGmAmAmAmAmAmGmUmG | |
| | GAAAAAGUGGCA | | mGmCmAmCmCmGmAmGmU | |
| | CCGAGUCGGUGC | | mCmGmGmUmGmCmU*mU*m | |
| | UUUU | | U*mU | |
| G028716 | CGUGCUCAACUG | 121 | mC*mG*mU*GCUCAACUGCC | 221 |
| | CCAAGGGAGUUU | | AAGGGAGUUUUAGAmGmC | |
| | UAGAGCUAGAAA | | mUmAmGmAmAmAmUmAmG | |
| | UAGCAAGUUAAA | | mCAAGUUAAAAUAAGGCUA | |
| | AUAAGGCUAGUC | | GUCCGUUAUCACGAAAGGG | |
| | CGUUAUCACGAA | | CACCGAGUCGGmU*mG*mC* | |
| | AGGGCACCGAGU | | mU | |
| | CGGUGCU | | | |
| G028718 | UACCCCUCCACG | 122 | mU*mA*mC*CCCUCCACGGU | 222 |
| | GUACCGGGGUUU | | ACCGGGGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCACGAAAGGGC | |
| | CGUUAUCACGAA | | ACCGAGUCGGmU*mG*mC* | |
| | AGGGCACCGAGU | | mU | |
| | CGGUGCU | | | |
| G028717 | CAGCACACUCGG | 123 | mC*mA*mG*CACACUCGGGG | 223 |
| | GGCCUACAGUUU | | CCUACAGUUUUAGAmGmCm | |
| | UAGAGCUAGAAA | | UmAmGmAmAmAmUmAmGm | |
| | UAGCAAGUUAAA | | CAAGUUAAAAUAAGGCUAG | |
| | AUAAGGCUAGUC | | UCCGUUAUCACGAAAGGGC | |
| | CGUUAUCACGAA | | ACCGAGUCGGmU*mG*mC* | |
| | AGGGCACCGAGU | | mU | |
| | CGGUGCU | | | |

Within the above tables, in the context of an unmodified sequence, A, C, G, U, and N are, independently, any natural or non-natural adenine, cytosine, guanine, uridine, and any nucleotide (e.g., A, C, G, or U), respectively. In the context of a modified sequence, m is indicative of s 2′-O-methyl modified nucleotide; * is indicative of a phosphorothioate internucleotide linkage; and A, C, G, U, and N are RNA nucleotides, i.e., 2′-OH and phosphodiesterase linkage to the 3′ nucleotide, when present.

In some embodiments, a composition comprising one or more guide RNAs (gRNA) comprising guide sequences that direct an RNA-guided DNA binding agent, which can be a nuclease (e.g., a Cas nuclease such as Cas9, such as SpyCas9 cleavase), to a target DNA sequence in PCSK9 is provided. In some embodiments, an engineered cell comprising a genetic modification in a human PCSK9 sequence within genomic coordinates of chr1: 55039548 . . . 55064852 is provided. In some embodiments, an engineered cell comprising a genetic modification in a human PCSK9 sequence is provided, wherein the genetic modification comprises a modification of at least one nucleotide within the genomic coordinates corresponding to PCSK9 guide sequence selected from SEQ ID NOs: 1-20, optionally SEQ ID NOs: 1, 2, 7, 9, 13-15, 17, 18, or 20, optionally SEQ ID NOs: 9, 14, or 18. In some embodiments, an engineered cell comprising a genetic modification in a human PCSK9 sequence is provided, wherein the genetic modification comprises a modification of at least one nucleotide within the genomic coordinates selected from Table 1.

In some embodiments comprising a gRNA, the gRNA may comprise a crRNA comprising a guide sequence shown in Table 1 as a guide sequence. In some embodiments, the gRNA comprises a guide sequence shown in Table 1, e.g., as an sgRNA. In some embodiments, the gRNA may comprise a guide sequence selected from SEQ ID NOs: 1, 2, 7, 9, 13-15, 17, 18, or 20. In some embodiments, the gRNA may comprise a guide sequence selected from SEQ ID NOs: 9, 14, or 18.

The gRNA may comprise a guide sequence comprising 16, 17, preferably 18, 19, or 20 contiguous nucleotides of a guide sequence shown in Table 1, e.g., SEQ ID NOs: 1, 2, 7, 9, 13-15, 17, 18, or 20, optionally SEQ ID NOs: 9, 14, or 18. In some embodiments, the gRNA comprises a guide sequence with at least 80%, 85%, preferably 90%, or 95%, or 100% identity to a guide sequence shown in Table 1 SEQ ID NOs: 1, 2, 7, 9, 13-15, 17, 18, or 20, optionally SEQ ID NOs: 9, 14, or 18. In each embodiment described herein, the gRNA may comprise a crRNA and trRNA associated as a single RNA (sgRNA) or on separate RNAs (dgRNA). In the context of sgRNAs, the crRNA and trRNA components may be covalently linked, e.g., via a phosphodiester bond or other covalent bond.

In each embodiment described herein, the gRNA may comprise a crRNA and trRNA associated as a single RNA (sgRNA) or on separate RNAs (dgRNA). In the context of sgRNAs, the crRNA and trRNA components may be covalently linked, e.g., via a phosphodiester bond or other covalent bond.

In each embodiment described herein, the guide RNA may comprise two non-covalently linked RNA strands as a “dual guide RNA” or “dgRNA.” The dgRNA comprises a first RNA molecule comprising a crRNA comprising, e.g., a guide sequence shown in Table 1, and a second RNA molecule comprising a trRNA. The first and second RNA molecules may not be covalently linked, but may form an RNA duplex via the base pairing between portions of the crRNA and the trRNA.

In each embodiment described herein, the guide RNA may comprise a single RNA molecule as a “single guide RNA” or “sgRNA”. The sgRNA may comprise a crRNA (or a portion thereof) comprising a guide sequence shown in Table 1, or a guide sequence selected from SEQ ID NOs: 1, 2, 7, 9, 13-15, 17, 18, or 20, optionally SEQ ID NOs: 9, 14, or 18 covalently linked to a trRNA.

The sgRNA may comprise 16, 17, preferably 18, 19, or 20 contiguous nucleotides of a guide sequence shown in Table 1, or a guide sequence selected from SEQ ID NOs: 1, 2, 7, 9, 13-15, 17, 18, or 20, optionally SEQ ID NOs: 9, 14, or 18. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 55, 60, 65, 70, 75, 80, 90, 100, or more than 100 nucleotides. In some embodiments, the trRNA may comprise certain secondary structures, such as, for example, one or more hairpin or stem-loop structures, or one or more bulge structures.

In some embodiments, a composition comprising one or more guide RNAs comprising a guide sequence of any one of SEQ ID NOs: 1-20, optionally SEQ ID NOs: 1, 2, 7, 9, 13-15, 17, 18, or 20, optionally SEQ ID NOs: 9, 14, or 18 is provided.

In some embodiments, a composition comprising one or more sgRNAs comprising any one of SEQ ID NOs: 1, 2, 7, 9, 13-15, 17, 18, or 20, optionally SEQ ID NOs: 9, 14, or 18 is provided.

In one aspect, a composition comprising a gRNA that comprises a guide sequence that is at least 90% or 95% identical to any of the nucleic acids of SEQ ID NOs 1-20, is provided. In some embodiments, a composition comprising a gRNA that comprises a guide sequence that is at least 90% or 95% identical to any of SEQ ID NOs: 1-20, optionally SEQ ID NOs: 1, 2, 7, 9, 13-15, 17, 18, or 20, optionally SEQ ID NOs: 9, 14, or 18 is provided.

In some embodiments, a composition is provided comprising at least one, e.g., at least two gRNAs, comprising guide sequences selected from any one or two or more of the guide sequences of SEQ ID NOs: 1-20, optionally SEQ ID NOs: 1, 2, 7, 9, 13-15, 17, 18, or 20, optionally SEQ ID NOs: 9, 14, or 18. In some embodiments, the composition comprises at least two gRNAs that each comprise a guide sequence that is at least 90% or 95% identical to any of the nucleic acids of SEQ ID NOs: 1-20, optionally SEQ ID NOs: 1, 2, 7, 9, 13-15, 17, 18, or 20, optionally SEQ ID NOs: 9, 14, or 18.

The guide RNA compositions provided herein are designed to recognize (e.g., hybridize to) a target sequence in a PCSK9 gene. For example, the PCSK9 target sequence may be recognized and cleaved by a provided Cas cleavase comprising a guide RNA. In some embodiments, an RNA-guided DNA binding agent, such as a Cas cleavase, e.g., a SpyCas9 cleavase, may be directed by a guide RNA to a target sequence of a PCSK9 gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within a PCSK9 gene.

Without being bound by any particular theory, mutations (e.g., frameshift mutations resulting from indels, i.e., insertions or deletions, occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary to or having complementarity to a target sequence within PCSK9 is used to direct the RNA-guided DNA binding agent to a particular location in the appropriate PCSK9 gene.

In some embodiments, the Spy guide sequence is at least 90% or 95%; or 100% identical to the reverse complement of a target sequence present in a human PCSK9 gene. In some embodiments, the target sequence is complementary to the guide sequence of the guide RNA. In some embodiments, the degree of complementarity or identity between a guide sequence of a Spy guide RNA and its corresponding target sequence is at least 80%, 85%, preferably 90%, or 95%; or 100%. In some embodiments, the target sequence and the guide sequence of the Spy gRNA may be 100% complementary or identical.

In some embodiments, the target sequence and the guide sequence of the Spy gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, the Spy guide sequence comprises a sequence of at least 16, 17, preferably 18, 19, or 20 contiguous nucleotides of a sequence selected from SEQ ID NOs: 1-20, optionally SEQ ID NOs: 1, 2, 7, 9, 13-15, 17, 18, or 20, optionally SEQ ID NOs: 9, 14, or 18.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA that is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) modification or replacement of a naturally occurring nucleobase, including with a non-canonical nucleobase (an exemplary base modification); and (iv) modification of nucleotides at the 3′ end or 5′ end of the oligonucleotide, e.g., to provide exonuclease stability, e.g., with 2′ O-me, 2′ halide, or 2′ deoxy substituted ribose; or inverted abasic terminal nucleotide, or replacement of phosphodiester with phosphothioate.

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In certain embodiments, phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, 10%, 15%, preferably at least 20%, 25%, 30%, 35%, 40%, 45%, or 50%) of the positions in a modified gRNA are modified nucleosides or nucleotides. In some embodiments, at least 5% of the positions in the modified guide RNA are modified nucleotides or nucleosides. In some embodiments, at least 10% of the positions in the modified guide RNA are modified nucleotides or nucleosides. In some embodiments at least 15% of the positions in the modified gRNA are modified nucleotides or nucleosides. In some embodiments, preferably at least 20% of the positions in the modified gRNA are modified nucleotides or nucleosides. In some embodiments, no more than 65% of the positions in the modified gRNA are modified nucleotides. In some embodiments, no more than 55% of the positions in the modified gRNA are modified nucleotides. In some embodiments, no more than 50% of the positions in the modified gRNA are modified nucleotides. In some embodiments, 10-70% of the positions in the modified gRNA are modified nucleotides. In some embodiments, 20-70% of the positions in the modified gRNA are modified nucleotides. In some embodiments, 20-80% of the positions in the modified gRNA are modified nucleotides. In some embodiments, 20-50% of the positions in the modified gRNA are modified nucleotides and the nuclease is a Spy Cas9 nuclease.

Unmodified nucleic acids can be prone to degradation by, e.g., intracellular nucleases or nucleases found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, borano phosphate esters, methyl phosphonates, phosphoroamidates, phosphodithioate, alkyl or aryl phosphonates, and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications, e.g., an amide linkage. In some embodiments, the charged phosphate group can be replaced by a neutral moiety. Examples of moieties which can replace the phosphate group can include, without limitation, e.g., methyl phosphonate, carboxymethyl, carbamate, amide, and thioether. Further examples of moieties which can replace the phosphate group can include, without limitation, e.g., ethylene oxide linker, sulfonate, sulfonamide, thioformacetal, formacetal, methyleneimino, methylenemethylimino, methylenehydrazo, methylenedimethylhydrazo, and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine, and peptide nucleic acid (PNA) nucleoside surrogates.

The modified nucleosides and modified nucleotides can include one or more modifications to the sugar group, i.e., a sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative). 2′ modifications can include hydrogen (i.e. deoxyribose sugars); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g., L- nucleosides. As used herein, a single abasic sugar is not understood to result in a discontinuity of a duplex.

In certain embodiments, 2′ modifications include, for example, 2′-OMe, 2′-F, or 2′-H, optionally 2′-O-Me.

The modified nucleosides and modified nucleotides described herein, which can be incorporated into a modified nucleic acid, can include a modified base, also called a modified nucleobase. Examples of nucleobases include, but are not limited to, adenine (A), guanine (G), cytosine (C), and uridine (U). These nucleobases can be modified or wholly replaced to provide modified residues that can be incorporated into modified nucleic acids. The nucleobase of the nucleotide can be independently selected from a purine, a pyrimidine, a purine analog, or a pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the sgRNA may be chemically modified throughout. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the modification patterns disclosed in WO2018/107028, the contents of which are herein incorporated by reference in their entirety. In some embodiments, the guide RNAs disclosed herein comprise one of the structures/modification patterns disclosed in US20170114334, the contents of which are herein incorporated by reference in their entirety. In some embodiments, the guide RNAs disclosed herein comprise one of the structures/modification patterns disclosed in WO2017/136794, the contents of which are herein incorporated by reference in their entirety. In some embodiments, the guide RNAs disclosed herein comprise one of the structures/modification patterns disclosed in WO2019/237069, the contents of which are herein incorporated by reference in their entirety. In some embodiments, the guide RNAs disclosed herein comprise one of the structures/modification patterns disclosed in WO2021/119275, the contents of which are herein incorporated by reference in their entirety.

In some embodiments, the sgRNA comprises any of the modification patterns shown herein, where N is any natural or non-natural nucleotide, and wherein the totality of the N's comprise a PCSK9 guide sequence as described herein in Table 1. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 601), wherein the totality of N's comprise a PCSK9 guide sequence as described in Table 1, for example, where the N's are replaced with any of the guide sequences disclosed herein in Table 1, optionally wherein the N's are replaced with SEQ ID NOs: 1-20, optionally SEQ ID NOs: 1, 2, 7, 9, 13-15, 17, 18, or 20, optionally SEQ ID NOs: 9, 14, or 18.

In some embodiments, the sgRNA comprises any of the modification patterns shown herein, where N is any natural or non-natural nucleotide, and wherein the totality of the N's comprise a PCSK9 guide sequence as described herein in Table 1. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG G*mU*mG*mC (SEQ ID NO: 607), wherein the totality of N's comprise a PCSK9 guide sequence as described in Table 1, for example, where the N's are replaced with any of the guide sequences disclosed herein in Table 1, optionally wherein the N's are replaced with PCSK9 no 1-20, optionally SEQ ID NOs: 1, 2, 7, 9, 13-15, 17, 18, or 20, optionally SEQ ID NOs: 9, 14, or 18.

In some embodiments, the sgRNA comprises any of the modification patterns shown herein, where N is any natural or non-natural nucleotide, and wherein the totality of the N's comprise a PCSK9 guide sequence as described herein in Table 1. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCACGAAAGGGCACCGAGUCG GmU*mG*mC*mU (SEQ ID NO: 612), wherein the totality of N's comprise a PCSK9 guide sequence as described in Table 1, for example, where the N's are replaced with any of the guide sequences disclosed herein in Table 1, optionally wherein the N's are replaced with SEQ ID NOs: 1-20, optionally SEQ ID NOs: 1, 2, 7, 9, 13-15, 17, 18, or 20, optionally SEQ ID NOs: 9, 14, or 18.

Any of the modifications described below may be present in the gRNAs and mRNAs described herein.

In the context of chemically modified sequences, “A,” “C,” “G,” “N,” and “U” denote an RNA nucleotide, i.e., 2′-OH with a phosphodiesterase linkage to the 3′ nucleotide.

The terms “mA,” “mC,” “mU,” or “mG” are used to denote an adenine, cytosine, uridine, or guanidine nucleotide, respectively, that has been modified with 2′-O-Me.

Modification of 2′-Omethyl can be depicted as follows:

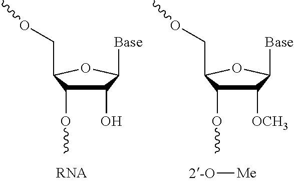

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” are used to denote a nucleotide that has been substituted with 2′-F.

Substitution of 2′-F can be depicted as follows:

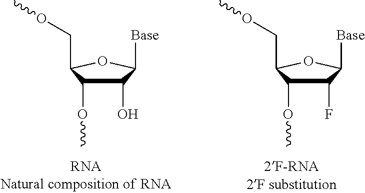

Phosphorothioate (PS) linkage or bond refers to a bond where a sulfur is substituted for one non-bridging phosphate oxygen in a phosphodiester linkage, for example in the bonds between nucleotide bases. When phosphorothioates are used to generate oligonucleotides, the modified oligonucleotides may also be referred to as S-oligos.

A “*” is used to denote a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” are used to denote a nucleotide that has been substituted with 2′-O-Me and that is linked to the next (e.g., 3′) nucleotide with a PS bond.

The diagram below shows the substitution of S— into a non-bridging phosphate oxygen, generating a PS bond in lieu of a phosphodiester bond:

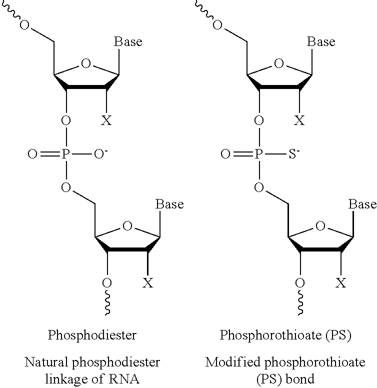

Abasic nucleotides refer to those which lack nitrogenous bases. The figure below depicts an oligonucleotide with an abasic (also known as apurinic) site that lacks a base. As used herein, the presence of a single abasic site is not considered to disrupt a duplex, e.g., a duplex formed between the targeting sequence of a guide RNA and a target site in the genome:

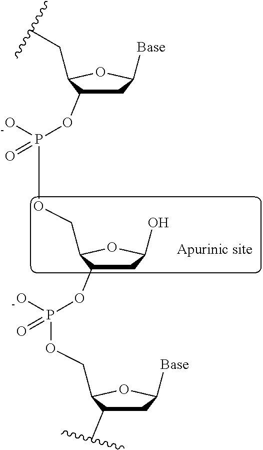

Inverted bases refer to those with linkages that are inverted from the normal 5′ to 3′ linkage (i.e., either a 5′ to 5′ linkage or a 3′ to 3′ linkage). Such inverted bases can only be present as a terminal nucleotide. In chemical synthesis methods performed 3′ to 5′, inverted bases do not have 5′ hydroxy available to grow the chain. For example:

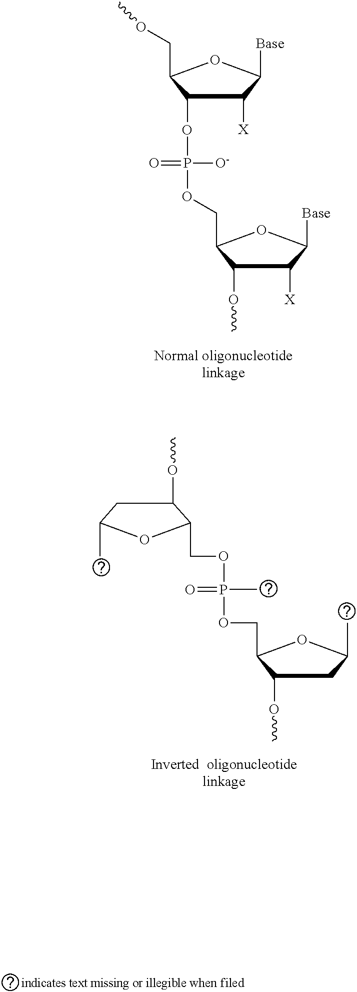

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 601), where N is any natural or non-natural nucleotide, and where the totality of the N's comprise a guide sequence that directs a nuclease to a target sequence in PCSK9, e.g., the genomic coordinates shown in Table 1, e.g., SEQ ID NOs: 1-20, optionally SEQ ID NOs: 1, 2, 7, 9, 13-15, 17, 18, or 20, optionally SEQ ID NOs: 9, 14, or 18.

In some embodiments, the guide RNA comprises a sgRNA comprising any one of the guide sequences of SEQ ID NOs: 1-20, optionally SEQ ID NOs: 1, 2, 7, 9, 13-15, 17, 18, or 20, optionally SEQ ID NOs: 9, 14, or 18 and a conserved portion of an sgRNA, for example, the conserved portion of sgRNA shown as Exemplary SpyCas9 sgRNA-1 or the conserved portions of the gRNAs shown in Tables 3-4 and throughout the specification. In some embodiments, the guide RNA comprises a sgRNA comprising any one of the guide sequences of SEQ ID NOs: 1-20, optionally SEQ ID NOs: 1, 2, 7, 9, 13-15, 17, 18, or 20, optionally SEQ ID NOs: 9, 14, or 18 and the nucleotides of GUUUUAGAGCUAGAAAUAGCAAGUUAAAAUAAGGCUAGUCCGUUAUCAACUU GAAAAAGUGGCACCGAGUCGGUGCUUUU (SEQ ID NO: 302) wherein the nucleotides are on the 3′ end of the guide sequence, and wherein the sgRNA may be modified as shown herein or in the sequence mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 601). In some embodiments, the sgRNA comprises Exemplary SpyCas9 sgRNA-1 or the modified versions thereof provided herein, or a version as provided in Table 3B or 4B, where the totality of the N's comprise a guide sequence that directs a nuclease to a target sequence. Each N is independently modified or unmodified. In certain embodiments, in the absence of an indication of a modification, the nucleotide is an unmodified RNA nucleotide residue, i.e., a ribose sugar and a phosphodiester backbone.

TABLE 3A
Exemplary unmodified Spy Cas9
scaffold sequences

| | | SEQ |
|---|---|---|
| | | ID |
| #mer | Unmodified nucleotide sequence | NO |
| | GUUUUAGAGCUAUGCUGUUUUG | 301 |
| 100 | GUUUUAGAGCUAGAAAUAGCAAGUUAAAAUAAG | 302 |
| | GCUAGUCCGUUAUCAACUUGAAAAAGUGGCACCG | |
| | AGUCGGUGCUUUU | |
| 96 | GUUUUAGAGCUAGAAAUAGCAAGUUAAAAUAAG | 303 |
| | GCUAGUCCGUUAUCAACUUGAAAAAGUGGCACCG | |
| | AGUCGGUGC | |
| 97 | GUUUUAGAGCUAGAAAUAGCAAGUUAAAAUAAG | 304 |
| | GCUAGUCCGUUAUCAACUUGAAAAAGUGGCACCG | |
| | AGUCGGUGCU | |
| 88 | GUUUUAGAGCUAGAAAUAGCAAGUUAAAAUAAG | 305 |
| | GCUAGUCCGUUAUCAACUUGGCACCGAGUCGGUG | |
| | C | |
| 88 | GUUUUAGAGCUAGAAAUAGCAAGUUAAAAUAAG | 306 |
| | GCUAGUCCGUUAUCAAAAUGGCACCGAGUCGGUG | |
| | C | |
| 88 | GGUUUUAGAGCUAGAAAUAGCAAGUUAAAAUAA | 307 |
| | GGCUAGUCCGUUAUCAAAAUGGCACCGAGUCGGU | |
| | GC | |
| 90 | GUUUUAGAGCUAGAAAUAGCAAGUUAAAAUAAG | 308 |
| | GCUAGUCCGUUAUCACGAAAGGGCACCGAGUCGG | |
| | UGC | |
| 91 | GUUUUAGAGCUAGAAAUAGCAAGUUAAAAUAAG | 309 |
| | GCUAGUCCGUUAUCACGAAAGGGCACCGAGUCGG | |
| | UGCU | |

TABLE 3B
Exemplary unmodified Spy Cas9
guide RNA sequences

| | | SEQ |
|---|---|---|
| | | ID |
| #mer | Unmodified nucleotide sequence | NO |
| 100 | (N)20GUUUUAGAGCUAGAAAUAGCAAGUUAAAAU | 401 |
| | AAGGCUAGUCCGUUAUCAACUUGAAAAAGUGGC | |
| | ACCGAGUCGGUGCUUUU | |
| 96 | (N)20GUUUUAGAGCUAGAAAUAGCAAGUUAAAAU | 402 |
| | AAGGCUAGUCCGUUAUCAACUUGAAAAAGUGGC | |
| | ACCGAGUCGGUGC | |
| 97 | (N)20GUUUUAGAGCUAGAAAUAGCAAGUUAAAAU | 403 |
| | AAGGCUAGUCCGUUAUCAACUUGAAAAAGUGGC | |
| | ACCGAGUCGGUGCU | |
| 88 | (N)20GUUUUAGAGCUAGAAAUAGCAAGUUAAAAU | 404 |
| | AAGGCUAGUCCGUUAUCAACUUGGCACCGAGUCG | |
| | GUGC | |
| 88 | (N)20GUUUUAGAGCUAGAAAUAGCAAGUUAAAAU | 405 |
| | AAGGCUAGUCCGUUAUCAAAAUGGCACCGAGUCG | |
| | GUGC | |
| 88 | (N)20GGUUUUAGAGCUAGAAAUAGCAAGUUAAAA | 406 |
| | UAAGGCUAGUCCGUUAUCAAAAUGGCACCGAGUC | |
| | GGUGC | |
| 90 | (N)20GUUUUAGAGCUAGAAAUAGCAAGUUAAAAU | 407 |
| | AAGGCUAGUCCGUUAUCACGAAAGGGCACCGAGU | |
| | CGGUGC | |
| 91 | (N)20GUUUUAGAGCUAGAAAUAGCAAGUUAAAAU | 408 |
| | AAGGCUAGUCCGUUAUCACGAAAGGGCACCGAGU | |
| | CGGUGCU | |

Wherein the Ns collectively are a guide sequence provided herein. Within the table, in the context of an unmodified sequence, A, C, G, U, and N are, independently, any natural or non-natural adenine, cytosine, guanine, uridine, and any nucleotide (e.g., A, C, G, or U), respectively.

TABLE 4A
Exemplary modified Spy Cas9 guide
scaffold sequences

| | | SEQ |
|---|---|---|
| | | ID |
| #mer | Modified sequence | NO |
| 100 | GUUUUAGAmGmCmUmAmGmAmAmAmUmAmGm | 501 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU*mU*mU*m | |
| | U | |
| 96 | GUUUUAGAmGmCmUmAmGmAmAmAmUmAmGm | 502 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmC | |
| 97 | GUUUUAGAmGmCmUmAmGmAmAmAmUmAmGm | 503 |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU | |
| 88 | GUUUUAGAmGmCmUmAmGmAmAmAmUmAmGm | 504 |
| | CAAGUUAAAAUAAGGCUAGUCCGUUAUCAACU | |
| | UGGCACCGAGUCGG*mU*mG*mC | |
| 88 | GUUUUAGAmGmCmUmAmGmAmAmAmUmAmGm | 505 |
| | CAAGUUAAAAUAAGGCUAGUCCGUUAUCAAAA | |
| | UGGCACCGAGUCGG*mU*mG*mC | |
| 88 | GUUUUAGAmGmCmUmAmGmAmAmAmUmAmGm | 506 |
| | CAAGUUAAAAUAAGGCUAGUCCGUUAUCAmAm | |
| | AmAmUmGmGmCmAmCmCmGmAmGmUmCmGmG | |
| | *mU*mG*mC | |
| 90 | GUUUUAGAmGmCmUmAmGmAmAmAmUmAmGm | 507 |
| | CAAGUUAAAAUAAGGCUAGUCCGUUAUCACGA | |
| | AAGGGCACCGAGUCGG*mU*mG*mC | |
| 90 | GUUUUAGAmGmCmUmAmGmAmAmAmUmAmGm | 508 |
| | CAAGUUAAAAUAAGGCUAGUCCGUUAUCAmCm | |
| | GmAmAmAmGmGmGmCmAmCmCmGmAmGmUmC | |
| | mGmG*mU*mG*mC | |
| 91 | GUUUUAGAmGmCmUmAmGmAmAmAmUmAmGm | 509 |
| | CAAGUUAAAAUAAGGCUAGUCCGUUAUCACGA | |
| | AAGGGCACCGAGUCGGU*mG*mC*mU | |
| 91 | GUUUUAGAmGmCmUmAmGmAmAmAmUmAmGm | 510 |
| | CAAGUUAAAAUAAGGCUAGUCCGUUAUCACGA | |
| | AAGGGCACCGAGUCGGmUmGmC*mU | |
| 91 | GUUUfUAGmAmGmCmUmAmGmAmAmAmUmAmG | 511 |
| | mCmAmAGUfUmAfAmAfAmUAmAmGmGmCmUmA | |
| | GUmCmCGUfUAmUmCAmCmGmAmAmAmGmGmG | |
| | mCmAmCmCmGmAmGmUmCmGmGmU*mG*mC*m | |
| | U | |
| 91 | GUUUUAGAmGmCmUmAmGmAmAmAmUmAmGm | 512 |
| | CAAGUUAAAAUAAGGCUAGUCCGUUAUCACGA | |
| | AAGGGCACCGAGUCGGmU*mG*mC*mU | |

TABLE 4B
Exemplary modified Spy Cas9
guide sequences

| | | SEQ |
|---|---|---|
| | | ID |
| #mer | Modified sequence | NO |
| 100 | mN*mN*mN*NNNNNNNNNNNNNNNNNGUUUUAG | 601 |
| | AmGmCmUmAmGmAmAmAmUmAmGmCAAGUUA | |
| | AAAUAAGGCUAGUCCGUUAUCAmAmCmUmUmG | |
| | mAmAmAmAmAmGmUmGmGmCmAmCmCmGmAm | |
| | GmUmCmGmGmUmGmCmU*mU*mU*mU | |
| 96 | mN*mN*mN*NNNNNNNNNNNNNNNNN | 602 |
| | GUUUUAGAmGmCmUmAmGmAmAmAmUmAmGm | |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmC | |
| 97 | mN*mN*mN*NNNNNNNNNNNNNNNNN | 603 |
| | GUUUUAGAmGmCmUmAmGmAmAmAmUmAmGm | |
| | CAAGUUAAAAUAAGGCUAGUCCGUUAUCAmAm | |
| | CmUmUmGmAmAmAmAmAmGmUmGmGmCmAmC | |
| | mCmGmAmGmUmCmGmGmUmGmCmU | |
| 88 | mN*mN*mN*NNNNNNNNNNNNNNNNN | 604 |
| | GUUUUAGAmGmCmUmAmGmAmAmAmUmAmGm | |
| | CAAGUUAAAAUAAGGCUAGUCCGUUAUCAACU | |
| | UGGCACCGAGUCGG*mU*mG*mC | |
| 88 | mN*mN*mN*NNNNNNNNNNNNNNNNN | 605 |
| | GUUUUAGAmGmCmUmAmGmAmAmAmUmAmGm | |
| | CAAGUUAAAAUAAGGCUAGUCCGUUAUCAAAA | |
| | UGGCACCGAGUCGG*mU*mG*mC | |
| 88 | mN*mN*mN*NNNNNNNNNNNNNNNNN | 606 |
| | GUUUUAGAmGmCmUmAmGmAmAmAmUmAmGm | |
| | CAAGUUAAAAUAAGGCUAGUCCGUUAUCAmAm | |
| | AmAmUmGmGmCmAmCmCmGmAmGmUmCmGmG | |
| | *mU*mG*mC | |
| 90 | mN*mN*mN*NNNNNNNNNNNNNNNNNGUUUUAG | 607 |
| | AmGmCmUmAmGmAmAmAmUmAmGmCAAGUUA | |
| | AAAUAAGGCUAGUCCGUUAUCACGAAAGGGCA | |
| | CCGAGUCGG*mU*mG*mC | |
| 90 | mN*mN*mN*NNNNNNNNNNNNNNNNN | 608 |
| | GUUUUAGAmGmCmUmAmGmAmAmAmUmAmGm | |
| | CAAGUUAAAAUAAGGCUAGUCCGUUAUCAmCm | |
| | GmAmAmAmGmGmGmCmAmCmCmGmAmGmUmC | |
| | mGmG*mU*mG*mC | |
| 91 | mN*mN*mN*NNNNNNNNNNNNNNNNN | 609 |
| | GUUUUAGAmGmCmUmAmGmAmAmAmUmAmGm | |
| | CAAGUUAAAAUAAGGCUAGUCCGUUAUCACGA | |
| | AAGGGCACCGAGUCGGU*mG*mC*mU | |
| 91 | mN*mN*mN*NNNNNNNNNNNNNNNNN | 610 |
| | GUUUUAGAmGmCmUmAmGmAmAmAmUmAmGm | |
| | CAAGUUAAAAUAAGGCUAGUCCGUUAUCACGA | |
| | AAGGGCACCGAGUCGGmUmGmC*mU | |
| 91 | mN*mN*mN*NNNNNNNNNNNNNNNNN | 611 |
| | GUUUfUAGmAmGmCmUmAmGmAmAmAmUmAmG | |
| | mCmAmAGUfUmAfAmAfAmUAmAmGmGmCmUmA | |
| | GUmCmCGUfUAmUmCAmCmGmAmAmAmGmGmG | |
| | mCmAmCmCmGmAmGmUmCmGmGmU*mG*mC*m | |
| | U | |
| 91 | mN*mN*mN*NNNNNNNNNNNNNNNNNGUUUUAG | 612 |
| | AmGmCmUmAmGmAmAmAmUmAmGmCAAGUUA | |
| | AAAUAAGGCUAGUCCGUUAUCACGAAAGGGCA | |
| | CCGAGUCGGmU*mG*mC*mU | |

wherein “m” indicates a 2′-O-Me modification, “f” indicates a 2′-fluoro modification, a “*” indicates a phosphorothioate linkage between nucleotides, and no modification in the context of a modified sequence indicates an RNA (2′-OH) and a phosphodiesterase linkage to the 3′ nucleotide when one is present.

In certain embodiments, the chemically modified scaffold sequences of Table 4A further comprise a chemically modified targeting sequence. In certain embodiments, the chemically modified guide sequence is (mN*)3(N)13-17. In certain embodiments, the guide sequence is (mN*)3(N)17, i.e., mN*mN*mN*NNNNNNNNNNNNNNNNN. In certain embodiments, each N of the (N)13-17 or the (N)17 is unmodified. In certain embodiments, the each N in the (N)13-17 or the (N)17 is independently modified, e.g., independently modified with a 2′-O-methyl modification.

As noted above, in some embodiments, a composition or formulation disclosed herein comprises an mRNA comprising an open reading frame (ORF) encoding an RNA-guided DNA binding agent, such as a Cas nuclease, e.g., Cas9 nuclease, as described in Table 23. In some embodiments, an mRNA comprising an ORF encoding an RNA-guided DNA binding agent, such as a Cas nuclease, e.g., Cas9 nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the mRNA or modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g., with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111 (33): 12025-30; and Abbas et al. (2017) Proc Natl Acad Sci USA 114 (11): E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl (3′-O-methyl) GpppG and 7-methyl (3′deoxy) GpppG,” RNA 7:1486-1495. The ARCA structure is shown below.

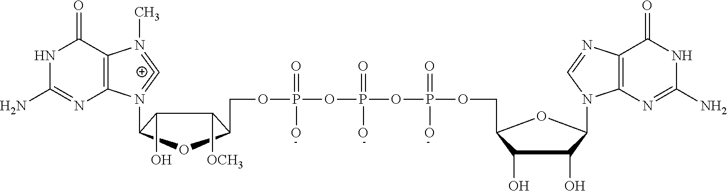

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively, or CleanCap AU: TriLink Biotechnologies as Cat. Nos. N-7114. The CleanCap™ AG structure is shown below.

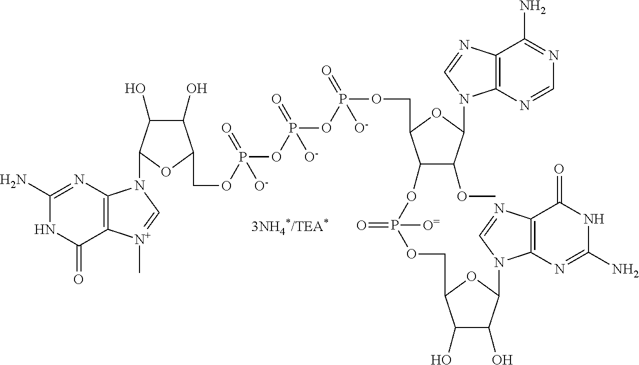

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; and Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides. In some embodiments, the poly-A tail includes non-adenine nucleotides, i.e., is an interrupted poly-A tail. In certain embodiments, the poly-A tail is interrupted by a non-adenine nucleotide about every 40, 50, 60, 70, 80, or 90 nucleotides. In certain embodiments, the poly-A tail is interrupted by a non-adenine nucleotide about every 50 nucleotides.

### Ribonucleoprotein Complex

In some embodiments, a composition is encompassed comprising one or more gRNAs comprising one or more guide sequences from Table 1 or one or more sgRNAs from Table 2 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, and other prokaryotes as known in the art, and modified (e.g., engineered or mutant) versions thereof.

In some embodiments, the Cas nuclease is the Cas9 nuclease from Streptococcus pyogenes. In some embodiments, the Cas nuclease is the Cas9 nuclease from Streptococcus pyogenes wherein the nuclease induces a double-strand break, i.e., is a cleavase.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the Cas nuclease is the Cas9 protein from the Spy CRISPR/Cas system. In some embodiments, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the wild type Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163 (3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Exemplary amino acid substitutions in the HNH or HNH-like nuclease domain or RuvC or RuvC-like domains for N. meningitidis include Nme2Cas9D16A (HNH nickase) and Nme2Cas9H588A (RuvC nickase).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US20140186958; US20150166980; and US20190338308.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-5 NLS(s). In some embodiments, the RNA-guided DNA-binding agent may be fused with 1, 2, or 3NLS(s). In some embodiments, the RNA-guided DNA-binding agent may be fused with two NLS(s). In some embodiments, the RNA-guided DNA-binding agent may be fused with one NLS. Where one NLS is used, the NLS may be linked at the N-terminus or the C-terminus of the RNA-guided DNA-binding agent sequence. In some embodiments, the NLS is not linked to the C-terminus. In some embodiments, the NLS is inserted within the RNA-guided DNA binding agent sequence. In certain circumstances, at least two NLSs comprised in the RNA-guided DNA-binding agent are the same (e.g., two SV40 NLSs). In certain embodiments, at least two different NLSs are present the RNA-guided DNA binding agent. In some embodiments, the RNA-guided DNA-binding agent is fused to two SV40 NLS sequences linked at the C-terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 1013) or PKKKRRV (SEQ ID NO: 1014). In some embodiments, the NLS may be a bipartite sequence, such as the NLS of nucleoplasmin, KRPAATKKAGQAKKKK (SEQ ID NO: 1015). In a specific embodiment, a single PKKKRKV NLS (SEQ ID NO: 1013) may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), orange fluorescent proteins (mOrange, mKO, Kusabira-Orange, Monomeric Kusabira-Orange, mTangerine, tdTomato), and any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly (NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, and fluorescent proteins.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); and Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase.

Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g., Cas9. In some embodiments, the gRNA is delivered to or expressed in a cell line that already stably expresses an RNA-guided DNA nuclease, such as a Cas nuclease or nickase, e.g., Cas9 nuclease or nickase. In some embodiments, the gRNA is delivered to a cell as part of an RNP. In some embodiments, the gRNA is delivered to a cell along with a mRNA encoding an RNA-guided DNA nuclease, such as a Cas nuclease or nickase, e.g. Cas9 nuclease or nickase.

As described herein, use of an RNA-guided DNA binding nuclease and a guide RNA disclosed herein can lead to double-strand breaks in the DNA which can produce errors in the form of insertion/deletion (indel) mutations upon repair by cellular machinery. Many mutations due to indels alter the reading frame or introduce premature stop codons and, therefore, produce a non-functional protein. In some embodiments, the efficacy of particular gRNAs is determined based on in vitro models. In some embodiments, the in vitro model is HEK293 cells stably expressing Cas9 (HEK293_Cas9). In some embodiments, the in vitro model is a primary cell line, e.g., a primary liver cell line, e.g., primary hepatocytes. In some embodiments, the primary hepatocytes are primary human hepatocytes. With respect to using primary cells, commercially available primary cells can be used to provide greater consistency between experiments. In some embodiments, the number of off-target sites at which a deletion or insertion occurs in an in vitro model (e.g., in a primary hepatocyte) is determined, e.g., by analyzing genomic DNA from cells transfected in vitro with Cas9 mRNA and the guide RNA. In some embodiments, such a determination comprises analyzing genomic DNA from the cells transfected in vitro with Cas9 mRNA, the guide RNA, and a donor oligonucleotide. Exemplary procedures for such determinations are provided in the working examples in which HEK293 cells or primary hepatocytes are used.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of a guide RNA is measured by percent indels or percent genetic modifications of PCSK9. In some embodiments, the efficacy of a guide RNA is measured by percent indels or percent genetic modifications at a PCSK9 locus. In some embodiments, the efficacy of a guide RNA is measured by percent indels or percent genetic modifications of PCSK9 at genomic coordinates of Table 1. In some embodiments, the percent editing of PCSK9 is compared to the percent indels or genetic modifications necessary to achieve reduction, e.g., knockdown, of the PCSK9 protein products. In some embodiments, the efficacy of a guide RNA is measured by reduced expression of PCSK9 protein. In embodiments, said reduced expression of PCSK9 protein is as measured by ELISA, e.g., as described herein.

In some embodiments, the PCSK9 protein expression is reduced in a population of cells using the methods and compositions disclosed herein. In some embodiments, the level of protein as determined, e.g., by ELISA, is reduced by at least 55%, 60%, 65%, 70%, 75%, preferably at least 80%, 85%, 90%, or 95% relative to a control population of unmodified cells.

An “unmodified cell” (or “unmodified cells”) refers to a control cell (or cells) of the same type of cell in an experiment or test, wherein the “unmodified” control cell has not been contacted with a PCSK9 guide. Therefore, an unmodified cell (or cells) may be a cell that has not been contacted with a guide RNA, or a cell that has been contacted with a guide RNA that does not target PCSK9.

In some embodiments, the efficacy of a guide RNA is measured by the number or frequency of indels or genetic modifications at off-target sequences within the genome of the target cell type, such as a primary hepatocyte cell. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., a primary hepatocyte cell), or which produce a frequency of off-target indel formation of <5% in a cell population or relative to the frequency of indel creation at the target site. In some embodiments, the disclosure provides guide RNAs which do not exhibit any off target indel formation in the target cell type (e.g., primary hepatocyte cell) as compared to a control cell. In some embodiments, guide RNAs are provided which produce indels at less than 5 validated off-target sites, e.g., as evaluated by one or more methods provided herein. In some embodiments, guide RNAs are provided which produce indels at less than or equal to 4, 3, 2, or 1 validated off-target site(s), e.g., as evaluated by one or more methods provided herein. In some embodiments, the off-target site(s) does not occur in a protein coding region in the target cell (e.g., hepatocyte) genome.

In some embodiments, the efficacy of a guide RNA is measured in vivo, e.g., in an animal or animal model having a DNA sequence susceptible to cleavage by a nuclease targeted by the guide RNA, i.e., having a DNA sequence sufficiently complementary to the targeting sequence in the guide RNA proximal to a cognate PAM for the guide and nuclease. In certain embodiments, the animal has an endogenous DNA sequence susceptible to cleavage by a nuclease targeted by the guide RNA. In certain embodiments, the animal model is a transgenic model, e.g., a mouse model having an inserted DNA sequence susceptible to cleavage by a nuclease targeted by the guide RNA, e.g., a mouse having an inserted human DNA sequence, e.g., a transgenic mouse having an inserted human PCSK9 sequence. The inserted sequence may or may not include one or more intron sequence or regulatory sequence, e.g., 3′ UTR, 5′ UTR, present in the human gene in its native context. In certain embodiments, the human DNA sequence may replace the homologous endogenous DNA sequence, e.g., the mouse PCSK9 gene is replaced by the human PCSK9 gene. In certain embodiments, the human gene is present in the mouse in the context of a human hepatocyte, e.g., a mouse with a humanized liver, e.g., as available from PheonixBio.

In some embodiments, the animal model is a rodent. In some embodiments, the rodent is a mouse or a rat. In some embodiments, the animal model is an animal expressing human PCSK9, e.g., a mouse expressing human PCSK9 from an expression construct, e.g., a viral vector, or a transgenic mouse expressing a human PCSK9. In some embodiments, the animal model is a high-fat fed, or hyperlipidimic animal, optionally an animal expressing a human PCSK9, e.g., a mouse expressing human PCSK9.

In some embodiments, detecting gene editing events, such as the formation of insertion/deletion (“indel”) mutations and insertion or homology directed repair (HDR) events in target DNA utilize linear amplification with a tagged primer and isolating the tagged amplification products (hereinafter referred to as “LAM-PCR,” or “Linear Amplification (LA)” method). In some embodiments, the efficacy of a guide RNA is measured by the levels of functional protein complexes comprising the expressed protein product of the gene. In some embodiments, the efficacy of a guide RNA is measured by ELISA.

### Genetic Modification for Inhibition of Target Gene Expression

Engineered cells or population of cells comprise a genetic modification, e.g., of an endogenous nucleic acid sequence encoding PCSK9.

In some embodiments, the engineered cells or population of cells comprise a genetic modification of a PCSK9 gene as assessed by sequencing, e.g., NGS, wherein at least 50%, 55%, 60%, 65%, 70%, 75%, preferably at least 80%, 85%, or 90% of cells comprise an insertion, deletion, or substitution in the endogenous PCSK9 sequence. In some embodiments, at least 50% of cells in the population comprise a modification selected from an insertion, a deletion, and a substitution in the endogenous PCSK9 sequence. In some embodiments, at least 80% of cells in the population comprise a modification selected from an insertion, a deletion, and a substitution in the endogenous PCSK9 sequence. In some embodiments, at least 85% of cells in the population comprise a modification selected from an insertion, a deletion, and a substitution in the endogenous PCSK9 sequence. In some embodiments, at least 90% of cells in the population comprise a modification selected from an insertion, a deletion, and a substitution in the endogenous PCSK9 sequence. In some embodiments, the cells in a population comprise hepatocytes in a liver. In some embodiments, PCSK9 expression is decreased by at least 50%, 55%, 60%, 65%, 70%, 75%, preferably at least 80%, 85%, or 90%, as compared to a suitable control, e.g., wherein the PCSK9 gene has not been modified. In some embodiments, expression of PCSK9 is decreased by at least 70%, or to below the limit of detection of the assay as compared to a suitable control, e.g., wherein the PCSK9 gene has not been modified. In some embodiments, expression of PCSK9 is decreased by at least 75%, or to below the limit of detection of the assay as compared to a suitable control, e.g., wherein the PCSK9 gene has not been modified. In some embodiments, expression of PCSK9 is decreased by at least 80%, or to below the limit of detection of the assay as compared to a suitable control, e.g., wherein the PCSK9 gene has not been modified. In some embodiments, expression of PCSK9 is decreased by at least 85%, or to below the limit of detection of the assay as compared to a suitable control, e.g., wherein the PCSK9 gene has not been modified. In some embodiments, expression of PCSK9 is decreased by at least 90%, or to below the limit of detection of the assay as compared to a suitable control, e.g., wherein the PCSK9 gene has not been modified. In some embodiments, expression of PCSK9 is decreased by no more than 95%, as compared to a suitable control, e.g., wherein the PCSK9 gene has not been modified. Assays for PCSK9 protein and mRNA expression are known in the art.

It is understood that in some embodiments, in vivo, the level of expression may be inhibited in one, but not in all, tissues where the target gene is expressed. For example, many genes are expressed predominantly in the liver, but may also be expressed in other tissues. In certain embodiments, the level of inhibition of expression may be for a particular tissue or cell type, but not inhibition of expression systemically, e.g., inhibition of hepatic expression rather than systemic expression. In certain embodiments, surrogate markers can be used to monitor changes in expression. For example, many proteins made in the liver are secreted into circulation; therefore, the level of inhibition of expression may be determined by or correlated with a decrease in levels of the protein in the blood. In certain embodiments, inhibition of expression in the liver can result in a change in a metabolite or other biomarker in a body fluid, e.g., blood or urine. The change in the level of the metabolite can be correlated with the level of inhibition of expression. Such correlations can be useful for monitoring the level of inhibition of expression in lieu of, e.g., serial biopsies which are not practical for monitoring in human subjects, or often in animal models. The level of inhibition of expression, or absolute level of a protein in blood or serum after treatment with an agent to reduce expression of a protein in the liver have been correlated with a therapeutic outcome.

In some embodiments, the target gene is genetically modified using a guide RNA with an RNA-guided DNA binding agent, resulting in inhibition of expression in a cell. In some embodiments, disclosed herein are cells engineered by inducing a break (e.g., double-strand break (DSB) or single-strand break (nick)) within target genes in the cells, e.g., using a guide RNA with an RNA-guided DNA-binding agent (e.g., a CRISPR/Cas system). The methods may be used in vitro, e.g., for screening guides, or in vivo, e.g., to provide a therapeutic benefit.

In some embodiments, the guide RNAs mediate a target-specific cutting by an RNA-guided DNA-binding agent (e.g., Cas nuclease, e.g., a SpyCas9 nuclease) at a site described herein within a target gene. It will be appreciated that, in some embodiments, the guide RNAs comprise guide sequences that bind to, or are capable of binding to, said regions.

### III. METHODS AND USES INCLUDING THERAPEUTIC METHODS AND USES OF GENOME EDITING AGENTS

The gRNAs and associated methods and compositions disclosed herein are useful for making genome editing therapeutic agents.

In some embodiments, the gRNAs comprising the guide sequences of Table 1 together with an RNA-guided DNA nuclease such as a Cas nuclease induce DSBs, and non-homologous end joining (NHEJ) during repair leads to a modification, e.g., a mutation, in a PCSK9 gene. In some embodiments, NHEJ leads to a deletion or insertion of a nucleotide(s), which induces a frame shift or nonsense mutation in a PCSK9 gene. In certain embodiments, gRNAs comprising guide sequences targeted to target genomic sequences are also delivered to the cell together with RNA-guided DNA nuclease such as a Cas nuclease, either together or separately, to make a genetic modification in a target genomic sequence to inhibit the expression of a full-length expression product from the target gene. In certain embodiments, the gRNAs are sgRNAs.

In some embodiments, the guide RNAs, compositions, and formulations are used to produce a cell in vivo, e.g., liver cell, e.g., a hepatocyte with a genetic modification in a PCSK9 gene. In some embodiments, the cell is in a subject.

In some embodiments, the subject is mammalian. In some embodiments, the subject is human. In some embodiments, the subject is a non-human primate.

In some embodiments, the subject has or is at risk of having a PCSK9-related disease or condition. A “PCSK9-associated disease or condition”, or a “PCSK9-related disease or condition” as used herein, is intended to include any disease or condition associated with the PCSK9 gene or protein expression or activity. Such a disease or condition may be caused, for example, by excess production of the PCSK9 protein, by PCSK9 gene mutations, by abnormal cleavage of the PCSK9 protein, by abnormal interactions between PCSK9 and other proteins or other endogenous or exogenous substances. Exemplary PCSK9-associated diseases include lipidemias, e.g., hyperlipidemias, and other forms of lipid imbalance such as hypercholesterolemia, hypertriglyceridemia and the pathological conditions associated with these disorders such as heart and circulatory diseases. In some embodiments, a PCSK9-related disease or condition is selected from the group consisting of a cardiovascular disease or a chronic liver injury. In some embodiments, a PCSK9-related disease or condition includes, but is not limited to, hypercholesterolemia (e.g., total blood cholesterol levels >190 mg/dl, or LDL-cholesterol levels >100 mg/dl), familial hypercholesterolemia (FH), autosomal dominant hypercholesterolemia (ADH), autosomal recessive hypercholesterolemia (ARH), hyperlipidemia, hypertriglyceridemia, coronary artery disease, stroke, myocardial infarction, obesity, xanthoma, atherosclerosis, aortic stenosis, liver steatosis, high blood pressure, type 2 diabetes, and insulin resistance.

Familial hypercholesterolemia (FH) is characterized by severely elevated LDL cholesterol (LDL-C) levels (e.g., over 190 mg/dL in adults, or over 160 mg/dL in children) that lead to atherosclerotic plaque deposition in the coronary arteries and proximal aorta at an early age, leading to an increased risk for cardiovascular disease, which may manifest as angina, myocardial infarction, or stroke. FH is a genetic disease that is passed on through family, and is caused by a pathogenic mutation in one of three genes (APOB, LDLR, and PCSK9). Patients can be heterozygous or homozygous for the mutation.

Autosomal dominant hypercholesterolemia (ADH) and autosomal recessive hypercholesterolemia (ARH) are other forms of hypercholesterolemia, also characterized by excessive blood cholesterol levels (Cohen, J. C, (2003) Curr. Opin. Lipidol. 14, 121-127). ADH is due to defects in LDL uptake by the liver, which may be caused by LDLR mutations that prevent LDL uptake, or by mutations in the protein on LDL, apolipoprotein B, which is responsible for LDL binding to LDLR. ARH is caused by mutations in the ARH protein that are necessary for endocytosis of the LDLR-LDL complex via its interaction with clathrin.

It is understood that subjects with a PCSK9-associated disease may be treated with one or more additional therapeutic agents within the standard of care for treatment of lipid disorders, or conditions associated with lipid disorders, e.g., cardiovascular disease, e.g., high blood pressure; type 2 diabetes, insulin resistance. In certain embodiments, the subject is treated with the additional agent until a reduction of serum PCSK9 level is observed. In certain embodiments, the subject is treated with an additional agent until a change is observed in a sign or symptom associated with the PCSK9-associated disease, e.g., a reduction of blood pressure prior to discontinuation of treatment with one or more agents to reduce hypertension; a normalization of blood sugar or blood sugar regulation prior to discontinuation of treatment with one or more agents to normalize blood sugar or blood sugar regulation. In some embodiments, the subject is treated with one or more PCSK9 inhibitors. In some embodiments, the subject is treated with one or more anti-PCSK9 monoclonal antibodies. In some embodiments, the subject is treated with evolocumab. In some embodiments, the subject is treated with alirocumab. In some embodiments, the subject is treated with vutrisiran.

Examples of additional therapeutic agents include those known to treat a lipid disorder, such as hypercholesterolemia, atherosclerosis or dyslipidemia. For example, a gRNA featured in the invention can be administered with, e.g., an HMG-COA reductase inhibitor (e.g., a statin), a fibrate, a bile acid sequestrant, niacin, an antiplatelet agent, an angiotensin converting enzyme inhibitor, an angiotensin II receptor antagonist (e.g., losartan potassium), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a glycoprotein Ilb/IIIa inhibitor, aspirin or an aspirinlike compound, an IB AT inhibitor, a squalene synthase inhibitor, or a monocyte chemoattractant protein (MCP)-I inhibitor. Exemplary HMG-COA reductase inhibitors include atorvastatin, pravastatin, simvastatin, lovastatin, fluvastatin, cerivastatin, rosuvastatin, and pitivastatin. Exemplary fibrates include, e.g., bezafibrate, clofibrate, fenofibrate, gemfibrozil, and ciprofibrate. Exemplary bile acid sequestrants include, e.g., cholestyramine, colestipol, and colesevelam. Exemplary niacin therapies include, e.g., immediate release and extended release formulation. Exemplary antiplatelet agents include, e.g., aspirin, clopidogrel, and ticlopidine. Exemplary angiotensin-converting enzyme inhibitors include, e.g., ramipril and enalapril. Exemplary acyl CoA cholesterol acetyltransferase (AC AT) inhibitors include, e.g., avasimibe and eflucimibe. Exemplary cholesterol absorption inhibitors include, e.g., ezetimibe and pamaqueside. Exemplary CETP inhibitors include, e.g., Torcetrapib, JTT-705, and CETi-I. Exemplary microsomal triglyceride transfer protein (MTTP) inhibitors include, e.g., implitapide, R-103757, and CP-346086.

Exemplary bile acid modulators include, e.g., HBS-107 (Hisamitsu/Banyu), Btg-511 (British Technology Group), BARI-1453 (Aventis), S-8921 (Shionogi), SD-5613 (Pfizer), and AZD-7806 (AstraZeneca). Exemplary peroxisome proliferation activated receptor (PPAR) agonists include, e.g., tesaglitazar, netoglitazone. Exemplary Glycoprotein Ilb/Illa inhibitors include, e.g., roxifiban, gantofiban, and cromafiban. The anti-atherosclerotic agent BO-653 (Chugai Pharmaceuticals), and the nicotinic acid derivative Nyclin are also appropriate for administering in combination with a gRNA featured in the invention. Exemplary combination therapies suitable for administration with a gRNA targeting PCSK9 include, e.g., advicor, amlodipine/atorvastatin, and ezetimibe/simvastatin. Agents for treating hypercholesterolemia, and suitable for administration in combination with a gRNA targeting PCSK9 include, e.g., lovastatin, amlodipine besylate, atorvastatin, rosuvastatin, fluvastatin, niacin, pravastatin, fenofibrate, ezetimibe, simvastatin, colesevelam, and ezetimibe.

In certain embodiments, methods comprise instructing an end user, e.g., a healthcare provider, a subject, to administer an additional agent, such as that provided above, in conjunction with administration of a gRNA provided herein. In certain embodiments, an additional agent, i.e., one or more additional agents, is administered in conjunction with, e.g., before, at the time of, or after administration of the gRNA, for example, until a desired clinical outcome is reached, e.g., reduction of blood pressure or serum cholesterol or lipid; normalization of blood sugar.

In one aspect, the invention provides a method of treating a patient by selecting a patient on the basis that the patient is in need of LDL lowering, LDL lowering without lowering of HDL, ApoB lowering, or total cholesterol lowering. The method includes administering to the patient a gRNA in an amount sufficient to lower the patient's LDL levels or ApoB levels, e.g., without substantially lowering HDL levels.

Genetic predisposition plays a role in the development of target gene associated diseases, e.g., hyperlipidemia. However, the lack of functional evidence for most variants detected during the molecular screening of patients with clinical familial hypercholesterolemia (FH) can make the definitive diagnosis difficult (see, e.g., Di Costanzo et al. (2021) J Clin Lipidol, 15:822-831). Therefore, a patient in need of a gRNA can be identified by taking a family history, or, for example, screening for one or more genetic markers or variants, typically in conjunction with, or prompted by, signs for hyperlipidemia. Examples of genes involved in hyperlipidemia include but are not limited to, e.g., LDL receptor (LDLR), the apoliproteins (ApoA1, ApoB, ApoE, and the like), Cholesteryl ester transfer protein (CETP), Lipoprotein lipase (LPL), hepatic lipase (LIPC), Endothelial lipase (EL), Lecithinxholesteryl acyltransferase (LCAT). It is expected that population based genomic studies, e.g., UK Biobank, will further define genetic markers associated with familial hypercholesterolemia and other hyperlipidemias.

A healthcare provider, such as a doctor, nurse, or geneticist can take a family history before prescribing or administering a gRNA agent of the invention. In addition, a test may be performed to determine a genotype or phenotype. For example, a DNA test may be performed on a sample from the patient, e.g., a blood sample, to identify the PCSK9 genotype or phenotype before a PCSK9 gRNA is administered to the patient. Variants in PCSK9, both pathogenic and benign, can be found, for example in the NCBI SNP database at www.ncbi.nlm.nih.gov/snp/?LinkName=gene_snp&from_uid=255738. In another embodiment, a test is performed to identify a related genotype or phenotype, e.g., an LDLR genotype. Examples of genetic variants with the LDLR gene can be found in the art, e.g., in the following publications which are incorporated by reference: Costanza et al (2005) Am J Epidemiol. 15; 161 (8): 714-24; Yamada et al. (2008) J Med Genet. January; 45 (1): 22-8, Epub 2007 Aug. 31; and Boes et al (2009) Exp. Gerontol 44:136-160, Epub 2008 Nov. 17.

Delivery of gRNA Compositions

Lipid nanoparticles (LNPs) are a well-known means for delivery of nucleotide and protein cargo, and may be used for delivery of the guide RNAs and compositions disclosed herein in vivo and in vitro. In some embodiments, the LNPs deliver nucleic acid cargo, protein cargo, or nucleic acid together with protein cargo.

In some embodiments, a method for delivering any one of the cells or populations of cells disclosed herein to a subject is provided, wherein the gRNA is delivered via an LNP in vivo. In some embodiments, the gRNA/LNP is also associated with a Cas9 or an mRNA encoding Cas9.

In some embodiments, a composition comprising any one of the gRNAs disclosed herein and an LNP is provided. In some embodiments, the composition further comprises a Cas9 or an mRNA encoding Cas9.

In some embodiments, LNPs associated with the gRNAs disclosed herein are for use in preparing a medicament for treating a disease or disorder.

In some embodiments, a method for delivering any one of the gRNAs disclosed herein in vivo is provided, wherein the gRNA is associated with an LNP. In some embodiments, the gRNA is not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle (LNP); see e.g., WO2017/173054 and WO2021/222287, the contents of each of which are herein incorporated by reference in their entirety.

In certain embodiments, DNA or RNA vectors encoding any of the guide RNAs comprising any one or more of the guide sequences described herein are provided. In some embodiments, in addition to guide RNA sequences, the vectors further comprise nucleic acids that do not encode guide RNAs. Nucleic acids that do not encode guide RNAs include, but are not limited to, promoters, enhancers, regulatory sequences, and nucleic acids encoding an RNA-guided DNA binding nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA binding nuclease, which can be a Cas nuclease, such as a Cas9 nuclease, such as a SpyCas9 cleavase. In some embodiments, the vector comprises one or more nucleotide sequence(s) encoding a crRNA, a trRNA, and an mRNA encoding an RNA-guided DNA binding nuclease, which can be a Cas protein, such as, Cas9. In one embodiment, the Cas9 is from Streptococcus pyogenes (i.e., Spy Cas9), for example a SpyCas9 cleavase. In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced into a cell as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid: nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained and tolerances accepted within the art. At the very least, and not as an attempt to limit the application of the doctrine of equivalents to the scope of the claims, each numerical parameter should at least be construed in light of the number of reported significant digits and by applying ordinary rounding techniques.

### IV. EXAMPLES

The following examples are provided to illustrate certain disclosed embodiments and are not to be construed as limiting the scope of this disclosure in any way.

### Example 1. Materials and Methods

In Vitro Transcription (“IVT”) of Nuclease mRNA

Capped and polyadenylated mRNA containing N1-methyl pseudo-U was generated by in vitro transcription using routine methods. Typically, a DNA plasmid containing a T7 promoter, a sequence for transcription, and a polyadenylation region was linearized with XbaI per manufacturer's protocol. The XbaI was inactivated by heating. The linearized plasmid was purified from enzyme and buffer salts. The IVT reaction to generate modified mRNA was performed by incubating at 37° C.: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase; 1 U/μL murine RNase inhibitor (NEB); 0.004 U/μL inorganic E. coli pyrophosphatase (NEB); and 1× reaction buffer. TURBO DNase (Thermo Fisher) was added to a final concentration of 0.01 U/μL, and the reaction was incubated at 37° C. to remove the DNA template.

The mRNA was purified using a MegaClear Transcription Clean-up kit (Thermo Fisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA was purified using LiCl precipitation, ammonium acetate precipitation, and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 el42). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanalyzer (Agilent).

Streptococcus pyogenes (“Spy”) Cas9 mRNA was generated from plasmid DNA encoding an open reading frame according to SEQ ID Nos: 1003, 1006, and 1009 (see sequences in Table 23). When the sequences cited in this paragraph are referred to below with respect to RNAs, it is understood that Ts should be replaced with Us (which can be modified nucleosides as described above). Messenger RNAs used in the Examples include a 5′ cap and a 3′ polyadenylation sequence, e.g., up to 100 nts. Guide RNAs were chemically synthesized by commercial vendors or using standard in vitro synthesis techniques with modified nucleotides.

Preparation of LNP Formulation Containing sgRNA and Cas9 mRNA

In general, the lipid nanoparticle components were dissolved in 100% ethanol at various molar ratios. The RNA cargos (e.g., Cas9 mRNA and sgRNA) were dissolved in 25 mM citrate, 100 mM NaCl, pH 5.0, resulting in a concentration of RNA cargo of approximately 0.45 mg/mL. The LNPs used contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate), also called herein Lipid A, cholesterol, 1,2-distearoyl-sn-glycero-3-phosphocholine (DSPC), and 1,2-dimyristoyl-rac-glycero-3-methoxypolyethylene glycol-2000 (PEG2K-DMG) (e.g., catalog #GM-020 from NOF, Tokyo, Japan) in a molar ratio of 50% Lipid A, 38% cholesterol, 9% DSPC, and 3% PEG2k-DMG. The LNPs were formulated with a lipid amine to RNA phosphate (N:P) molar ratio of about 6, and a ratio of gRNA to mRNA of 1:2 by weight. The LNPs used comprise a single RNA species such as Cas9 mRNA or a sgRNA. LNPs are similarly prepared with a mixture of Cas9 mRNA and a guide RNA.

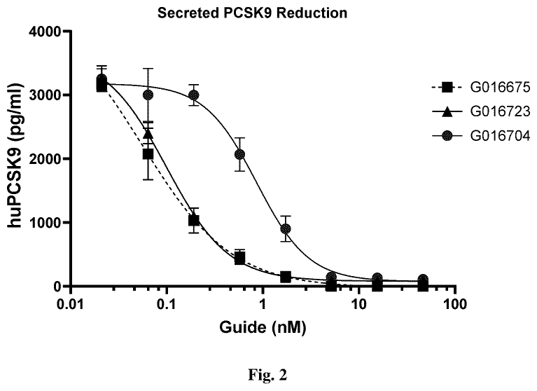

The LNPs were prepared using a cross-flow technique utilizing impinging jet mixing of the lipid in ethanol with two volumes of RNA solution and one volume of water. First, the lipid in ethanol was mixed through a mixing cross with the two volumes of RNA solution. Then, a fourth stream of water was mixed with the outlet stream of the cross through an inline tee (See WO2016010840 FIG. 2). The LNPs were held for 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNPs were characterized to determine the encapsulation efficiency, polydispersity index, and average particle size. The final LNP was stored at 4° C. or −80° C. until further use.

### Hepatocyte Cell Preparation

Primary human hepatocytes (PHH) and primary cynomolgus hepatocytes (PCH) were prepared as follows. Cells were thawed and resuspended in 50 mL Cryopreserved Hepatocyte Recovery Media (CHRM) (Invitrogen, CM7000) followed by centrifugation. Cells were resuspended in hepatocyte medium with plating supplements: William's E Medium Plating Supplements with fetal bovine serum (FBS) content (Gibco, Cat. A13450). Cells were pelleted by centrifugation, resuspended in media and plated on Bio-coat collagen I coated 96-well plates (Corning #354407). Plated cells were allowed to settle and adhere for 4-6 hours in a tissue culture incubator at 37° C. and 5% CO₂ atmosphere. After incubation cells were checked for monolayer formation and were washed once and plated with 100 μL hepatocyte maintenance medium: William's E Medium (Gibco, Cat. A12176-01) plus supplement pack (Gibco, Cat. CM3000).

### DNA Isolation

Cells were harvested post-transfection at 72 hours. DNA was extracted from each well of a 96-well plate using 50 μL/well QuickExtract DNA Extraction solution (Epicentre, Cat. QE09050) or Quick Extract (Lucigen, Cat. SS000035-D2) according to manufacturer's protocol. Alternatively, DNA was isolated by methods known in the art.

### Next-Generation Sequencing (“NGS”) and Analysis for Editing Efficiency

To quantitatively determine the efficiency of editing at the target location in the genome, sequencing was utilized to identify the presence of insertions and deletions introduced by gene editing. PCR primers were designed around the target site within the gene of interest (e.g., PCSK9), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion or deletion (“indel”) was calculated.

The editing percentage (e.g., the “editing efficiency,” “percent editing,”, or “percent indel”) is defined as the total number of sequence reads with insertions or deletions (“indels”) over the total number of sequence reads, including wild type.

### Example 2. In Vitro Editing in Primary Hepatocytes

sgRNAs respectively targeting the human PCSK9 gene with various targeting sequences were designed as shown in Table 1 and lipofected into primary human (PHH) hepatocytes. Lipofection of Cas9 mRNA and gRNAs used pre-mixed lipid formulations. The lipofection reagent contained ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate), also called herein Lipid A, cholesterol, DSPC, and PEG2k-DMG in a molar ratio of 50% Lipid A, 38% cholesterol, 9% DSPC, and 3% PEG2k-DMG. This mixture was reconstituted in 100% ethanol then mixed with RNA cargos (e.g., Cas9 mRNA (SEQ ID NO: 1002) and gRNA) at a lipid amine to RNA phosphate (N:P) molar ratio of about 6 to produce lipid nucleic acid mixtures. An mRNA comprising a Cas9 ORF of Table 23 was produced by in vitro transcription (IVT) as described in WO2019/067910, see e.g., ¶ 354, using a 2 hour IVT reaction time and purifying the mRNA by LiCl precipitation followed by tangential flow filtration.

PHH (Gibco, Lot #9396) cells were used and plated at densities of 40,000 and 33,000 cells/well, respectively. Lipofection samples were prepared using an N:P molar ratio of about 7 and a gRNA:mRNA ratio of 6.5:1 by weight. Cells were incubated at 37° C., 5% CO₂ for 24 hours prior to treatment with the lipid nucleic acid mixtures. Lipid nucleic acid mixtures were incubated in media containing 10% fetal bovine serum (FBS) at 37° C. for 10 minutes. Post-incubation, the lipid nucleic acid mixtures comprising 50 ng of Cas9 mRNA were added to the cells. The cells were lysed 72 hours post-treatment for NGS analysis as described in Example 1. Mean editing results with standard deviation (SD) are shown in Table 5 for PHH. Samples were run in duplicate.

TABLE 5
In vitro editing in PHH

| Guide ID | Mean % Edit | SD |
|---|---|---|
| G016707 | 75.50 | 4.4 |
| G016675 | 69.55 | 1.7 |
| G016723 | 61.35 | 6.8 |
| G016704 | 58.85 | 2.1 |
| G016649 | 55.00 | 1.4 |
| G016696 | 52.90 | 6.9 |
| G016735 | 52.50 | 6.5 |
| G016650 | 52.10 | 7.6 |
| G016714 | 50.25 | 7.7 |
| G016662 | 49.20 | 0.8 |
| G016689 | 48.70 | 2.2 |
| G016730 | 48.10 | 2.0 |
| G016660 | 47.05 | 11.6 |
| G016709 | 46.70 | 8.3 |
| G016674 | 45.15 | 7.9 |
| G016657 | 45.05 | 5.5 |
| G016687 | 43.25 | 4.9 |
| G016661 | 41.50 | 0.0 |
| G016690 | 41.00 | 4.5 |
| G016654 | 40.20 | 1.6 |

### Example 3. In Vitro Editing in Primary Hepatocytes with Dilution Curve

Guide RNAs targeting PCSK9 were tested for editing efficacy in primary human hepatocytes (PHH) (Gibco, Lot: Hu8284).

### Example 3.1. Cell Preparation

PHH were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells resuspended in hepatocyte plating medium plus supplement pack (Invitrogen, Cat. A1217601 and Gibco, Cat. CM3000). Cells were counted and plated with a density of 33,000 cells/well on Bio-coat collagen I coated 96-well plates (Thermo Fisher, Cat. 877272). Plated cells were allowed to settle and adhere for 4-6 hours in a tissue culture incubator at 37° C. and 5% CO2 atmosphere. After incubation cells were checked for monolayer formation and were washed once with hepatocyte maintenance medium (Invitrogen, Cat. A1217601 and Gibco, Cat. CM4000).

### Example 3.2. LNP Treatment

LNPs were generally prepared as described in Example 1. The LNPs contained 50% Lipid A, 38% cholesterol, 9% DSPC, and 3% PEG2k-DMG by molar ratio. The LNPs were formulated with a lipid amine to RNA phosphate (N:P) molar ratio of about 6, and a ratio of gRNA to mRNA (SEQ ID NO: 1002) of 1:2 by weight. Each LNP was applied to cells using an 8-point 3-fold dilution curve starting at 300 ng mRNA/100 μl as shown in Table 6.

TABLE 6
Concentrations of guide and mRNA for dose response curve

| Guide concentration (nM) | mRNA (ng) |
|---|---|
| 46.5 | 300 |
| 15.5 | 100 |
| 5.167 | 33.33 |
| 1.722 | 11.11 |
| 0.574 | 3.70 |
| 0.191 | 1.23 |
| 0.064 | 0.412 |
| 0.021 | 0.137 |

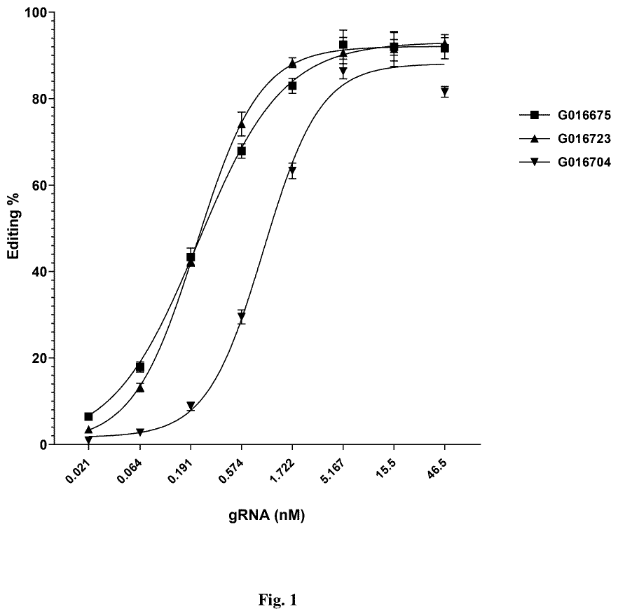

Upon treatment with LNPs, cells were incubated for 24 hours at 37° C. in William's E Medium (Gibco, A1217601) with maintenance supplements and 3% fetal bovine serum. After 72 hours, cells were harvested and analyzed by NGS as described in Example 1. EC50 values and mean editing results are shown in Table 7. Dose response curves are plotted in FIG. 1

TABLE 7
The editing efficiency and EC50 (nM) for selected PCSK9 guides

| Guide | G016675 | | | | G016723 | | | | G016704 | | | |
|---|---|---|---|---|---|---|---|---|---|---|---|---|
| concentration | Indel | | | EC50 | Indel | | | EC50 | Indel | | | EC50 |
| (nM) | (%) | SD | N | (nM) | (%) | SD | N | (nM) | (%) | SD | N | (nM) |
| 46.5 | 91.6 | 2.5 | 6 | 0.22 | 92.8 | 2.0 | 6 | 0.21 | 81.6 | 1.2 | 6 | 0.92 |
| 15.5 | 92.0 | 3.3 | 6 | | 91.5 | 4.0 | 6 | | 91.9 | 1.8 | 6 | |
| 5.167 | 92.5 | 3.4 | 6 | | 90.6 | 3.5 | 6 | | 86.3 | 1.8 | 6 | |
| 1.722 | 83.0 | 1.8 | 6 | | 88.3 | 1.1 | 6 | | 63.3 | 1.8 | 6 | |
| 0.574 | 67.9 | 1.7 | 6 | | 74.1 | 2.8 | 6 | | 29.5 | 1.6 | 6 | |
| 0.191 | 43.4 | 2.1 | 6 | | 42.4 | 0.9 | 6 | | 8.8 | 1.0 | 6 | |
| 0.064 | 17.9 | 1.2 | 6 | | 13.2 | 1.0 | 6 | | 2.7 | 0.6 | 6 | |
| 0.021 | 6.5 | 0.6 | 6 | | 3.5 | 0.5 | 6 | | 0.9 | 0.3 | 6 | |

### Example 3.3. Proprotein Convertase 9 (PCSK9) ELISA Analysis Used in PHH

The plated cells were cultured about 10 days with Cellartis Power Primary HEP Medium (Takara, Y20020). Media was changed every other day. Tissue culture media was collected 48 h after the last media change on day 10. Secreted PCSK9 serum levels were determined using a Human Proprotein Convertase 9 (PCSK9) DuoSet ELISA Kit (R&D systems, Cat. DY3888) according to the manufacturers' protocol using 2 μg/ml final concentration of capture antibody. The plates were read on a Clariostar plate reader at an absorbance of 450 nm and a wavelength correction of 570 nm. Serum PCSK9 levels were calculated by using a four-parameter logistic curve fit off the standard curve. Dose response curve for reduction of PCSK9 protein (pg/ml) are shown in FIG. 2, and the data are presented in Table 8. Final maximum protein reduction is shown in Table 9. Samples were run in triplicate. Percent protein knockdown (% KD) values were determined relative to untreated control group.

TABLE 8
Dose response curve for reduction of serum PCSK9

| Guide | G016675 | | | G016723 | | | G016704 | | |
|---|---|---|---|---|---|---|---|---|---|
| (nM) | Mean | SD | N | Mean | SD | N | Mean | SD | N |
| 46.5 | 0.0 | 0.0 | 3 | 81.5 | 6.7 | 3 | 108.5 | 21.1 | 3 |
| 15.5 | 0.0 | 0.0 | 3 | 81.2 | 4.7 | 3 | 128.8 | 29.4 | 3 |
| 5.167 | 0.0 | 0.0 | 3 | 83.5 | 5.3 | 3 | 144.9 | 26.2 | 3 |
| 1.722 | 155.0 | 69.8 | 3 | 138.0 | 43.5 | 3 | 900.0 | 199.3 | 3 |
| 0.574 | 459.2 | 116.6 | 3 | 434.3 | 58.6 | 3 | 2068.2 | 261.3 | 3 |
| 0.191 | 1031.0 | 194.8 | 3 | 1095.7 | 76.5 | 3 | 2997.3 | 164.1 | 3 |
| 0.064 | 2075.7 | 404.9 | 3 | 2400.8 | 164.9 | 3 | 3000.7 | 415.9 | 3 |
| 0.021 | 3165.8 | 87.3 | 3 | 3256.8 | 200.8 | 3 | 3253.2 | 158.3 | 3 |

TABLE 9
Max protein reduction of selected guides in PHH

| | Max protein | Protein reduction |
|---|---|---|
| Guide ID | reduction (% KD) | EC50 (nM) |
| G016675 | >95% | 0.06 |
| G016723 | >95% | 0.1 |
| G016704 | >95% | 0.9 |

### Example 4. In Vivo Editing in Mouse Liver Using Lipid Nanoparticles (LNPs)

The LNPs used in all in vivo studies were formulated as described in Example 1. Transport and storage solution (TSS) used in LNP preparation was dosed in the experiment as a vehicle-only negative control. The nucleotide sequences of the sgRNA contained in the LNPs each target a different sequence in the PCSK9 gene as indicated in Table 2.

### Example 4.1. In Vivo Editing in a Humanized PCSK9 Mouse Model

Selected guide designs from Table 2 were tested for editing efficiency in vivo. Male and female transgenic mice comprising a human PCSK9 gene sequence (hPCSK9) in their genomes, were used in each study involving mice. The hPCSK9 mice were generated on a hybrid C57B6/129 background, then backcrossed once to B6, and then intercrossed for cohort expansion. The hPCSK9 mice had the mouse PCSK9 gene excised out of their genomes. Animals were about 6 weeks old and were weighed pre-dose. LNPs were dosed via the lateral tail vein at 0.3 milligrams per kilogram body weight (e.g., 0.3 mg/kg, or 0.3 mpk). The animals were observed at approximately 24 hours post dose for adverse effects. Animals were euthanized at 14 days post dose by exsanguination under isoflurane anesthesia and cervical dislocation. Blood was collected via cardiac puncture into serum separator tubes or into tubes containing buffered sodium citrate for plasma as described herein. For studies involving in vivo editing, liver tissue was collected from the left lateral lobe of each animal for DNA extraction and analysis.

For the in vivo studies, genomic DNA was extracted from 10 mg of liver tissue using a bead-based extraction kit, e.g., the Zymo Quick-DNA 96 kit (Zymo Research, Cat. #D3010) according to the manufacturer's protocol, which includes homogenizing the tissue in lysis buffer (approximately 600 μL/10 mg tissue). All DNA samples were normalized to 100 ng/μL concentration for PCR and subsequent NGS analysis, as described in Example 1.

### Example 4.2. PCSK9 ELISA Analysis Used in Animal Studies

Blood was collected, and the serum was isolated as described above. The total PCSK9 serum levels were determined using a human PCSK9 ELISA Kit (Abcam, Cat. ab209884). Kit reagents and standards were prepared according to the manufacturer's protocol. Mouse serum was diluted between 5 to 10-fold. Both standard curve dilutions (100 μL each) and diluted serum samples were added to each well of the ELISA plate pre-coated with capture antibody. The plate was incubated at room temperature for 30 minutes before washing. Enzyme-antibody conjugate (100 μL per well) was added for a 20-minute incubation. Unbound antibody conjugate was removed, and the plate was washed again before the addition of the chromogenic substrate solution. The plate was incubated for 10 minutes before adding 100 μL of the stop solution, e.g., sulfuric acid (approximately 0.3 M). The plate was read on a SpectraMax M5 or Clariostar plate reader at an absorbance of 450 nm. Serum hPCSK9 levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to controls, which generally were animals sham-treated with vehicle (TSS) unless otherwise indicated.

### Example 4.3. In Vivo Editing and Serum hPCSK9 Knockdown

LNPs were generally prepared as described in Example 1. LNP formulations were analyzed for average particle size, polydispersity (pdi), total RNA content and encapsulation efficiency of RNA as described in Example 1.

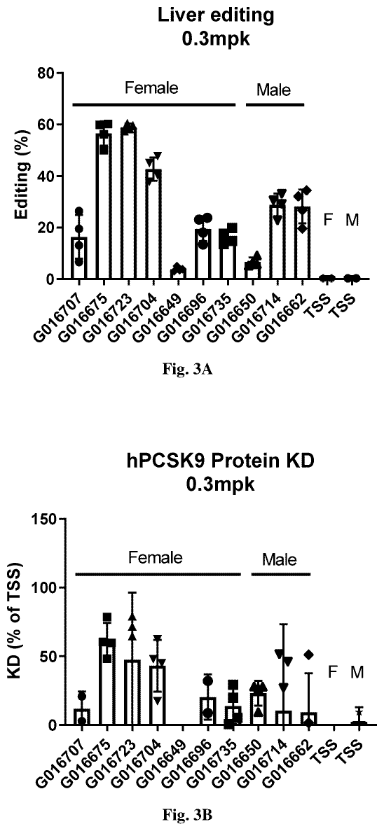

LNPs containing the sgRNAs indicated in Table 10 were administered to transgenic hPCSK9 mice (n=4 for all groups) at a dose of 0.3 mg/kg of animal body weight as described above. The editing efficiency, and percent hPCSK9 knockdown (% KD) compared to a TSS vehicle-only negative control for LNPs containing the indicated sgRNAs are shown in Table 10 and editing efficiency and hPCSK9 KD levels are illustrated in FIGS. 3A and 3B.

TABLE 10
Liver editing and hPCSK9 protein knockdown

| | | | Mean | |
|---|---|---|---|---|
| | Mean | | hPCSK9 | |
| Guide ID | % indel | SD | % KD | SD |
| TSS | 0.3 | 0.0 | 0.0 | 0.0 |
| TSS | 0.3 | 0.0 | 0.6 | 12.4 |
| G016707 | 16.4 | 8.5 | 11.8 | 12.7 |
| G016675 | 56.6 | 4.7 | 61.8 | 12.5 |
| G016723 | 59.0 | 1.4 | 47.5 | 49.0 |
| G016704 | 42.7 | 4.5 | 43.2 | 18.9 |
| G016649 | 3.9 | 0.9 | 0.0 | 66.9 |
| G016696 | 19.6 | 4.9 | 20.4 | 16.5 |
| G016735 | 16.5 | 2.8 | 13.8 | 13.2 |
| G016650 | 6.7 | 1.7 | 23.1 | 9.1 |
| G016714 | 28.8 | 4.4 | 10.4 | 62.9 |
| G016662 | 28.2 | 6.6 | 9.3 | 28.4 |

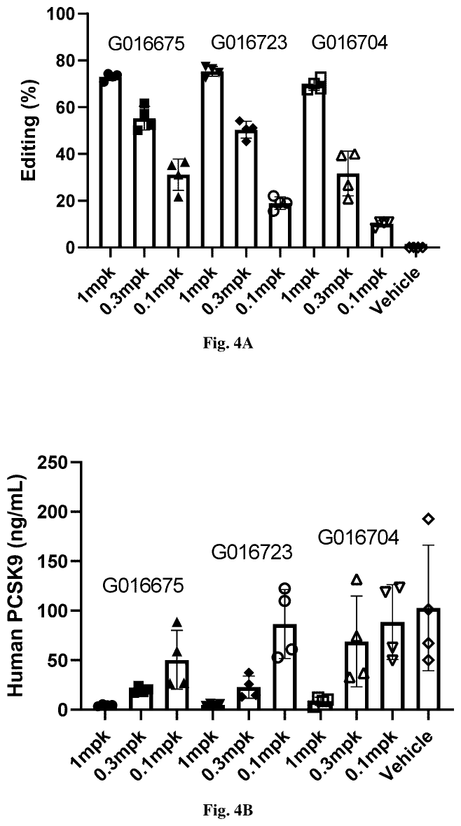

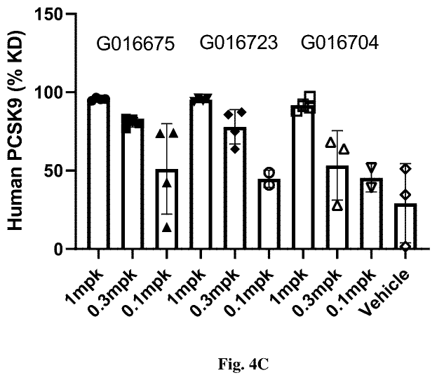

LNPs comprising selected guides from Table 10, G016675, G016723 and G016704, were administered to male and female transgenic hPCSK9 mice (n=2 male and n=2 female for each group) at 1 mg/kg, 0.3 mg/kg and 0.1 mg/kg of animal body weight as described above. Table 11 shows the editing efficiency, hPCSK9 protein levels, and percent hPCSK9 knockdown compared to a TSS vehicle-only negative control, respectively, for LNPs containing the indicated sgRNAs. Editing efficiency, hPCSK9 protein levels, and percent hPCSK9 KD levels are shown in FIGS. 4A-4C, respectively.

TABLE 11
Liver editing, serum hPCSK9 levels, and % KD serum hPCSK9

| | | | | Mean serum | | | |
|---|---|---|---|---|---|---|---|
| | Dose | Mean | | hPCSK9 | | Mean | |
| Guide | (mg/kg) | % indel | SD | (ng/ml) | SD | % KD | SD |
| TSS | 0 | 0.0 | 0.1 | 102.9 | 63.6 | 0 | 61.8 |
| G016675 | 1 | 73.0 | 1.5 | 4.5 | 0.8 | 95.6 | 0.8 |
| | 0.3 | 55.3 | 5.1 | 19.8 | 3.0 | 80.7 | 2.9 |
| | 0.1 | 31.1 | 6.7 | 50.3 | 29.7 | 51.1 | 28.9 |
| G016723 | 1 | 75.3 | 2.1 | 4.9 | 1.2 | 95.2 | 1.1 |
| | 0.3 | 50.3 | 3.6 | 22.8 | 11.2 | 77.9 | 10.9 |
| | 0.1 | 19.0 | 2.7 | 86.5 | 34.8 | 15.9 | 33.8 |
| G016704 | 1 | 69.6 | 2.4 | 8.6 | 4.0 | 91.7 | 3.9 |
| | 0.3 | 31.7 | 9.5 | 69.0 | 45.9 | 33.0 | 44.6 |
| | 0.1 | 10.1 | 1.3 | 88.6 | 37.8 | 13.9 | 36.7 |

### Example 5. Off-Target Analysis

### Example 5.1. Biochemical Off-Target Analysis

A biochemical method (See, e.g., Cameron et al., Nature Methods. 6, 600-606; 2017) was used to determine potential off-target genomic sites cleaved by Cas9 using specific guides targeting PCSK9. Single guide RNAs targeting human PCSK9 were screened using genomic DNA reference material NA24385 from the Coriell Institute alongside two control guides with known off-target profiles. The number of potential off-target sites was detected using a guide concentration of 48 nM and Cas9 protein concentration of 16 nM in the biochemical assay for which results are shown in Table 12.

TABLE 12
Biochemical Off-Target Analysis

| Guide ID | Target Gene | Sites |
|---|---|---|
| G016735 | PCSK9 | 5 |
| G016704 | PCSK9 | 11 |
| G016723 | PCSK9 | 28 |
| G016650 | PCSK9 | 31 |
| G016662 | PCSK9 | 31 |
| G016707 | PCSK9 | 33 |
| G016714 | PCSK9 | 55 |
| G016675 | PCSK9 | 77 |
| G016696 | PCSK9 | 87 |
| G016649 | PCSK9 | 152 |
| G000644 | EMX1 | 99 |
| G000645 | VEGFA | 1021 |

### Example 5.2. Targeted Sequencing for Validating Potential Off-Target Sites

Test guides were further evaluated for possible off-target indel formation using amplicon sequencing at potential off target sites following editing in cells. Each guide's respective potential off target sites were identified by biochemical assay described above or by in silico prediction.

In this experiment, 3 sgRNAs targeting human PCSK9 were evaluated in triplicates. Primary human hepatocytes (PHH, Gibco, Lot: Hu8284) were plated and transfected with LNPs comprising Cas9 mRNA and sgRNA. Each cell plate was treated via single dose transfection of 38.2 nM of guide (equivalent to 250 ng mRNA) to achieve dose saturation as required for further downstream off-target assays. DNA was isolated from the cells by lysing and subjected to NGS. Some potential off target sites failed quality metrics and are not counted in the “sites characterized” tally for Table 13. Repair structures were manually inspected at loci with statistically relevant indel rates at the off-target cleavage sites to confirm indel repair structures.

TABLE 13
Evaluation of potential off-target editing sites

| | Sites | Sites | Sites with statistically |
|---|---|---|---|
| Guide ID | evaluated | characterized | significant % indels |
| G016675 | 167 | 165 | 0 |
| G016723 | 142 | 137 | 2 |
| G016704 | 47 | 41 | 1 |

### Example 6. In Vitro Editing in Primary Hepatocytes with Dilution Curve

Guide RNAs targeting PCSK9 were tested for editing efficacy in primary cynomolgus hepatocytes (PCH) (Gibco, Lot: PCH-C423).

### Example 6.1. Cell Preparation

PCH were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells were resuspended in hepatocyte plating medium plus supplement pack (Invitrogen, Cat. A1217601 and Gibco, Cat. CM3000). Cells were counted and plated with a density of 40,000 cells/well on Bio-coat collagen I coated 96-well plates (Thermo Fisher, Cat. 877272). Plated cells were allowed to settle and adhere for 4-6 hours in a tissue culture incubator at 37° C. and 5% CO₂ atmosphere. After incubation cells were checked for monolayer formation and were washed once with hepatocyte maintenance medium (Invitrogen, Cat. A1217601 and Gibco, Cat. CM4000).

### Example 6.2. LNP Treatment

LNPs were generally prepared as described in Example 1. The LNPs contained 50% Lipid A, 38% cholesterol, 9% DSPC, and 3% PEG2k-DMG by molar ratio. The LNPs were formulated with a lipid amine to RNA phosphate (N:P) molar ratio of about 6, and a ratio of gRNA to mRNA (SEQ ID NO: 1002) of 1:2 by weight. Each LNP was applied to cells using an 8-point 3-fold dilution curve starting at 200 ng mRNA/100 μl as shown in Table 14.

TABLE 14
Concentrations of guide and mRNA for dose response curve

| Guide concentration (nM) | mRNA (ng) |
|---|---|
| 32.25 | 200 |
| 10.75 | 67 |
| 3.58 | 22.2 |
| 1.19 | 7.4 |
| 0.4 | 2.5 |
| 0.13 | 0.8 |
| 0.04 | 0.27 |
| 0.013 | 0.9 |

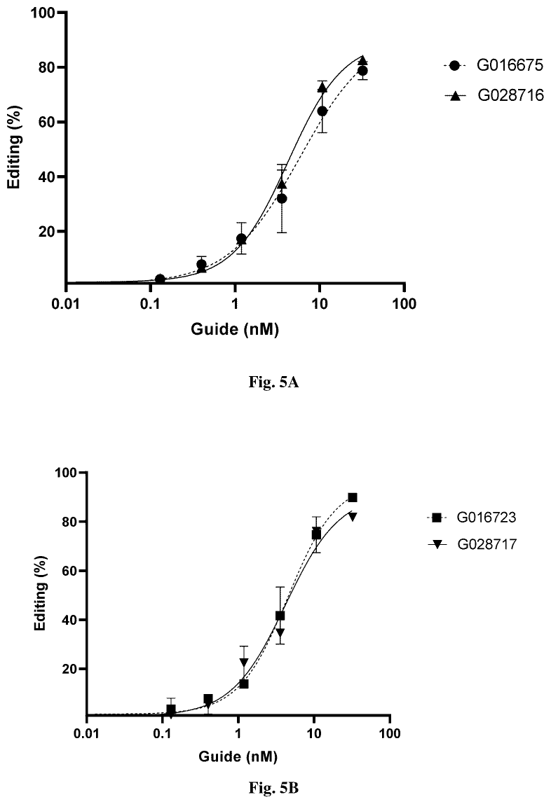

Upon treatment with LNPs, cells were incubated for 24 hours at 37° C. in William's E Medium (Gibco, A1217601) with maintenance supplements and 3% fetal bovine serum. Samples were run in duplicate. After 72 hours, cells were harvested and analyzed by NGS as described in Example 1. EC50 values and mean editing results are shown in Tables 15 and 16. Dose response curves are plotted in FIGS. 5A and 5B.

TABLE 15
The editing efficiency and EC50 (nM) for selected guides

| Guide | G016675 | | | | G028716 | | | |
|---|---|---|---|---|---|---|---|---|
| concentration | Indel | | | EC50 | Indel | | | EC50 |
| nM) | (%) | SD | N | (nM) | (%) | SD | N | (nM) |
| 32.25 | 78.8 | 3.3 | 2 | 5.85 | 82.7 | 0.2 | 2 | 4.26 |
| 10.75 | 64.0 | 7.9 | 2 | | 73.0 | 2.1 | 2 | |
| 3.58 | 32.1 | 12.5 | 2 | | 37.5 | 5.0 | 2 | |
| 1.19 | 17.5 | 5.7 | 2 | | 17.2 | 1.8 | 2 | |
| 0.4 | 8.0 | 2.9 | 2 | | 6.9 | 0.2 | 2 | |
| 0.13 | 2.6 | 0.9 | 2 | | 2.8 | 0.2 | 2 | |
| 0.04 | 0.4 | 0.1 | 2 | | 0.4 | 0.0 | 2 | |
| 0.013 | 0.3 | 0.0 | 2 | | 0.3 | 0.1 | 2 | |

TABLE 16
The editing efficiency and EC50 (nM) for selected guides

| Guide | G016723 | | | | G028717 | | | |
|---|---|---|---|---|---|---|---|---|
| concentration | Indel | | | EC50 | Indel | | | EC50 |
| (nM) | (%) | SD | N | (nM) | (%) | SD | N | (nM) |
| 32.25 | 89.9 | 0.4 | 2 | 4.48 | 81.8 | 1.5 | 2 | 4.20 |
| 10.75 | 74.7 | 7.3 | 2 | | 76.0 | 1.3 | 2 | |
| 3.58 | 41.7 | 11.7 | 2 | | 34.6 | 1.1 | 2 | |
| 1.19 | 13.8 | 1.4 | 2 | | 22.4 | 6.9 | 2 | |
| 0.4 | 7.9 | 0.5 | 2 | | 5.3 | 3.8 | 2 | |
| 0.13 | 3.6 | 4.5 | 2 | | 1.3 | 1.0 | 2 | |
| 0.04 | 0.1 | 0.0 | 2 | | 0.2 | 0.1 | 2 | |
| 0.013 | 0.1 | 0.0 | 2 | | 0.1 | 0.0 | 2 | |

### Example 7. In Vitro Editing in Primary Human Hepatocytes with Dilution Curve

Guide RNAs targeting PCSK9 were tested for editing efficacy in primary human hepatocytes (PHH) (Gibco/Thermo Fisher Lot: HU8381).

### Example 7.1. Cell Preparation

PHH were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells resuspended in hepatocyte plating medium plus supplement pack (Invitrogen, Cat. A1217601 and Gibco, Cat. CM3000). Cells were counted and plated with a density of 40,000 cells/well on Bio-coat collagen I coated 96-well plates (Thermo Fisher, Cat. 877272). Plated cells were allowed to settle and adhere for 4-6 hours in a tissue culture incubator at 37° C. and 5% CO2 atmosphere. After incubation cells were checked for monolayer formation and were washed once with hepatocyte maintenance medium (Invitrogen, Cat. A1217601 and Gibco, Cat. CM4000).

### Example 7.2. LNP Treatment

LNPs were generally prepared as described in Example 1. The LNPs contained 50% Lipid A, 38% cholesterol, 9% DSPC, and 3% PEG2k-DMG by molar ratio. The LNPs were formulated with a lipid amine to RNA phosphate (N:P) molar ratio of about 6, and a ratio of gRNA to mRNA (SEQ ID NO: 1002) of 1:2 by weight. Each LNP was applied to cells using an 8-point 3-fold dilution curve starting at 200 ng mRNA/100 μl as shown in Table 17.

TABLE 17
Concentrations of guide and mRNA for dose response curve.

| Guide concentration (nM) | mRNA (ng) |
|---|---|
| 32.25 | 200 |
| 10.75 | 67 |
| 3.58 | 22.2 |
| 1.19 | 7.4 |
| 0.4 | 2.5 |
| 0.13 | 0.8 |
| 0.04 | 0.27 |
| 0.013 | 0.9 |

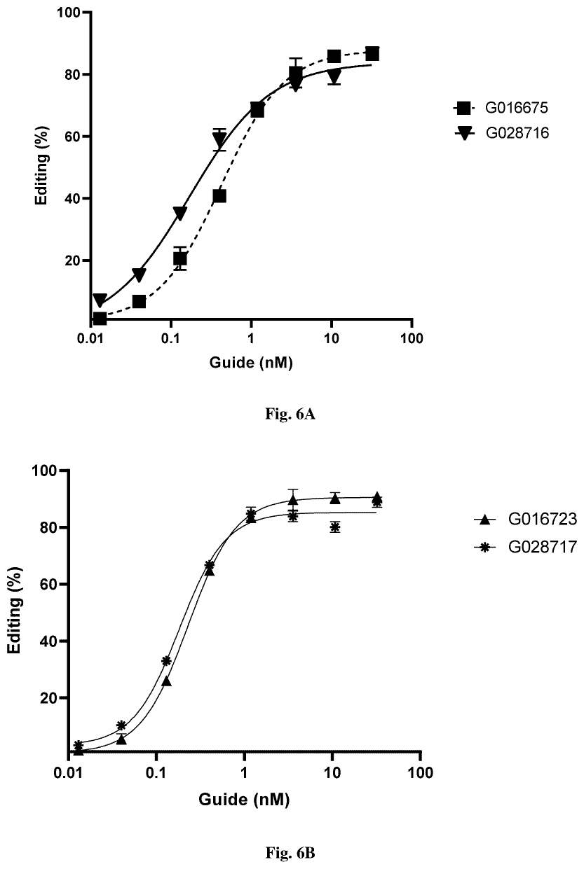

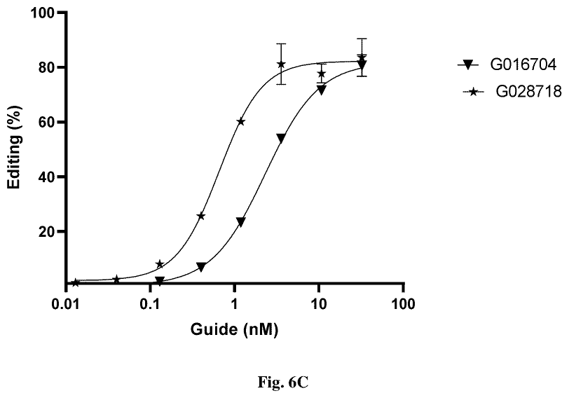

Upon treatment with LNPs, cells were incubated for 24 hours at 37° C. in William's E Medium (Gibco, A1217601) with maintenance supplements and 3% fetal bovine serum. Samples were run in duplicate. After 72 hours, cells were harvested and analyzed by NGS as described in Example 1. EC50 values and mean editing results are shown in Tables 18, 19 and 20. Dose response curves are plotted in FIGS. 6A-6C.

TABLE 18
The editing efficiency and EC50 (nM) for selected guides

| Guide | G016675 | | | | G028716 | | | |
|---|---|---|---|---|---|---|---|---|
| concentration | Indel | | | EC50 | Indel | | | EC50 |
| (nM) | (%) | SD | N | (nM) | (%) | SD | N | (nM) |
| 32.25 | 86.5 | 2.0 | 2 | 0.42 | 86.7 | 0.6 | 2 | 0.17 |
| 10.75 | 85.8 | 1.0 | 2 | | 79.1 | 2.3 | 2 | |
| 3.58 | 80.5 | 4.7 | 2 | | 76.5 | 1.3 | 2 | |
| 1.19 | 68.2 | 1.3 | 2 | | 68.9 | 0.6 | 2 | |
| 0.40 | 40.8 | 0.8 | 2 | | 58.9 | 3.5 | 2 | |
| 0.13 | 20.6 | 3.7 | 2 | | 35.0 | 2.1 | 2 | |
| 0.04 | 6.8 | 0.4 | 2 | | 15.1 | 0.9 | 2 | |
| 0.013 | 1.3 | 0.1 | 2 | | 7.0 | 0.5 | 2 | |

TABLE 19
The editing efficiency and EC50 (nM) for selected guides

| Guide | G016723 | | | | G028717 | | | |
|---|---|---|---|---|---|---|---|---|
| concentration | Indel | | | EC50 | Indel | | | EC50 |
| (nM) | (%) | SD | N | (nM) | (%) | SD | N | (nM) |
| 32.25 | 90.9 | 1.7 | 2 | 0.23 | 88.9 | 1.8 | 2 | 0.18 |
| 10.75 | 90.3 | 2.0 | 2 | | 80.2 | 1.9 | 2 | |
| 3.58 | 89.8 | 3.7 | 2 | | 83.9 | 1.9 | 2 | |
| 1.19 | 83.4 | 0.6 | 2 | | 84.9 | 2.3 | 2 | |
| 0.40 | 64.9 | 0.4 | 2 | | 66.8 | 1.6 | 2 | |
| 0.13 | 26.1 | 1.8 | 2 | | 33.0 | 0.1 | 2 | |
| 0.04 | 5.5 | 1.8 | 2 | | 10.3 | 0.1 | 2 | |
| 0.013 | 1.6 | 0.0 | 2 | | 3.4 | 0.1 | 2 | |

TABLE 20
The editing efficiency and EC50 (nM) for selected guides

| Guide | G016704 | | | | G028718 | | | |
|---|---|---|---|---|---|---|---|---|
| concentration | Indel | | | EC50 | Indel | | | EC50 |
| (nM) | (%) | SD | N | (nM) | (%) | SD | N | (nM) |
| 32.25 | 80.6 | 4.0 | 2 | 2.31 | 83.6 | 6.9 | 2 | 0.66 |
| 10.75 | 71.5 | 0.6 | 2 | | 77.8 | 3.5 | 2 | |
| 3.58 | 53.9 | 1.2 | 2 | | 81.1 | 7.4 | 2 | |
| 1.19 | 23.3 | 0.1 | 2 | | 60.1 | 1.0 | 2 | |
| 0.40 | 6.9 | 0.5 | 2 | | 25.7 | 1.2 | 2 | |
| 0.13 | 1.7 | 0.3 | 2 | | 8.1 | 0.9 | 2 | |
| 0.04 | 0.7 | 0.1 | 2 | | 2.5 | 0.6 | 2 | |
| 0.013 | 0.5 | 0.1 | 2 | | 1.3 | 0.1 | 2 | |

### Example 8. In Vitro Editing in Primary Human Hepatocytes with Dilution Curve

Guide RNAs targeting PCSK9 synthesized as two different guide formats were tested for editing efficacy in primary human hepatocytes (PHH) (Gibco/Thermo Fisher, Lot: HU8300, HU8373A, HU8284).

### Example 8.1. PHH Cell Preparation

PHH were thawed and resuspended in hepatocyte thawing medium with plating supplements (William's E Medium (Gibco, Cat. A12176-01)) with dexamethasone+cocktail supplement (Gibco, Cat. A15563, Lot 2019842) and Plating Supplements with FBS content (Gibco, Cat. A13450, Lot 1970698) followed by centrifugation. The supernatant was discarded, and the pelleted cells were resuspended in hepatocyte plating medium plus supplement pack (Invitrogen, Cat. A1217601 and Gibco, Cat. CM3000). Cells were counted and plated with a density of 33,000 cells/well on Bio-coat collagen I coated 96-well plates (Thermo Fisher, Cat. 877272). Plated cells were allowed to settle and adhere for 4-6 hours in a tissue culture incubator at 37° C. and 5% CO₂ atmosphere. After incubation cells were checked for monolayer formation and were washed once with hepatocyte maintenance medium (Invitrogen, Cat. A1217601 and Gibco, Cat. CM4000).

### Example 8.2. LNP Treatment and Editing

LNPs were generally prepared as described in Example 1. The LNPs contained 50% Lipid A, 38% cholesterol, 9% DSPC, and 3% PEG2k-DMG by molar ratio. The LNPs were formulated with a lipid amine to RNA phosphate (N:P) molar ratio of about 6, and a ratio of gRNA to mRNA (SEQ ID NO: 1002) of 1:2 by weight. Each LNP was applied to cells using a 12-point dose response curve starting with a LNP dose of 450 ng total RNA by weight.

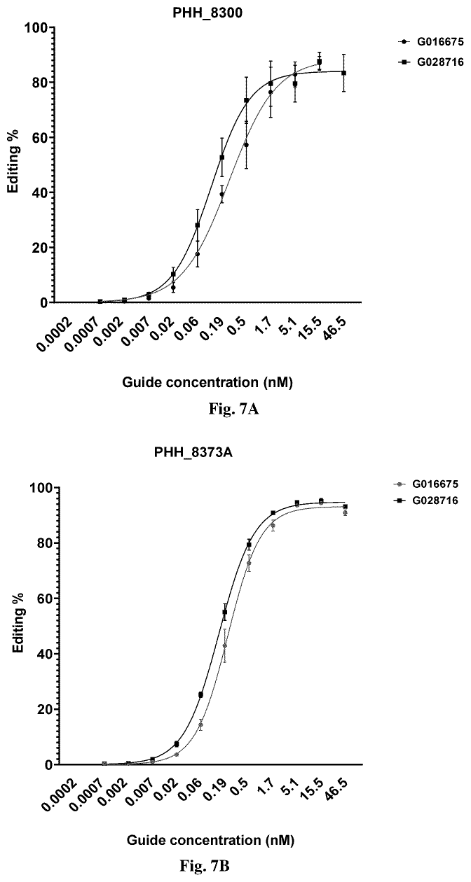

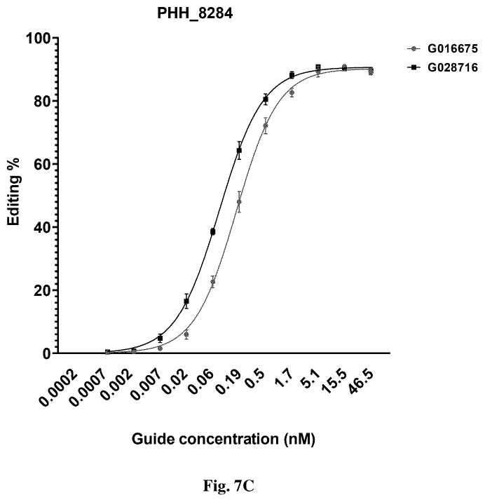

Upon treatment with LNPs, cells were incubated for 24 hours at 37° C. in William's E Medium (Gibco, A1217601) with maintenance supplements and 3% fetal bovine serum. Samples were run in duplicate. After 72 hours, cells were harvested and analyzed by NGS as described in Example 1. Dose response curves are plotted in FIGS. 7A-7C. EC50 values and mean percent editing results are shown in Tables 21A-21C.

TABLE 21A
Editing efficiency and EC50 (nM) for
selected guides in PHH Lot: HU8300

| Guide | | | | | | | | |
|---|---|---|---|---|---|---|---|---|
| Concen- | G016675 | | | | G028716 | | | |
| tration | Editing | | | EC50 | Editing | | | EC50 |
| (nM) | (%) | SD | N | (nM) | (%) | SD | N | (nM) |
| 46.55 | 80.75* | 9.4* | 4* | 0.27 | 83.4 | 6.7 | 4 | 0.12 |
| 15.51 | 87.1 | 2.4 | 4 | | 87.7 | 3.3 | 4 | |
| 5.17 | 82.9 | 4.6 | 4 | | 79.5 | 6.7 | 4 | |
| 1.72 | 76.4 | 9.2 | 4 | | 79.5 | 8.2 | 4 | |
| 0.57 | 57.3 | 8.6 | 4 | | 73.5 | 8.4 | 4 | |
| 0.19 | 39.4 | 3.1 | 4 | | 52.8 | 7.0 | 4 | |
| 0.064 | 17.6 | 4.6 | 4 | | 28.1 | 5.7 | 4 | |
| 0.021 | 5.4 | 1.8 | 4 | | 10.2 | 2.5 | 4 | |
| 0.0071 | 1.5 | 0.6 | 4 | | 2.8 | 0.9 | 4 | |
| 0.0024 | 0.4 | 0.2 | 4 | | 0.9 | 0.4 | 4 | |
| 0.0007 | 0.2 | 0.1 | 4 | | 0.3 | 0.2 | 4 | |
| 0.0002 | 0.3 | 0.1 | 4 | | 0.3 | 0.1 | 4 | |

The (*) editing results were excluded from EC50 calculation since the percent editing was significantly lower (≥5% difference) than the dose resulting in max editing.

TABLE 21B
Editing efficiency and EC50 (nM) for
selected guides in PHH Lot: HU8373A

| Guide | G016675 | | | | G028716 | | | |
|---|---|---|---|---|---|---|---|---|
| Concentration | Editing | | | EC50 | Editing | | | EC50 |
| (nM) | (%) | SD | N | (nM) | (%) | SD | N | (nM) |
| 46.55 | 90.9 | 1 | 4 | 0.22 | 93.2 | 0 | 4 | 0.15 |
| 15.51 | 94.4 | 0 | 4 | | 95.1 | 1 | 4 | |
| 5.17 | 93.6 | 0 | 4 | | 94.7 | 0 | 4 | |
| 1.72 | 86.3 | 2 | 4 | | 90.9 | 0 | 4 | |
| 0.57 | 72.7 | 3 | 4 | | 79.4 | 2 | 4 | |
| 0.19 | 42.9 | 6 | 4 | | 55.1 | 3 | 4 | |
| 0.064 | 14.4 | 2 | 4 | | 25.3 | 1 | 4 | |
| 0.021 | 3.6 | 0 | 4 | | 7.5 | 1 | 4 | |
| 0.0071 | 0.9 | 0 | 4 | | 1.9 | 0 | 4 | |
| 0.0024 | 0.3 | 0 | 4 | | 0.5 | 0 | 4 | |
| 0.0007 | 0.3 | 0 | 4 | | 0.4 | 0 | 4 | |
| 0.0002 | 0.3 | 0 | 4 | | 0.4 | 0 | 4 | |

TABLE 21C
Editing efficiency and EC50 (nM) for
selected guides in PHH Lot: HU8284

| Guide | G016675 | | | | G028716 | | | |
|---|---|---|---|---|---|---|---|---|
| Concentration | Editing | | | EC50 | Editing | | | EC50 |
| (nM) | (%) | SD | N | (nM) | (%) | SD | N | (nM) |
| 46.55 | 89.3 | 1.1 | 4 | 0.17 | 89.6 | 1.1 | 4 | 0.08 |
| 15.51 | 90.9 | 0.6 | 4 | | 90.3 | 0.5 | 4 | |
| 5.17 | 89.3 | 1.7 | 4 | | 90.8 | 0.6 | 4 | |
| 1.72 | 82.6 | 1.3 | 4 | | 88.3 | 1.0 | 4 | |
| 0.57 | 72.2 | 2.5 | 4 | | 80.5 | 1.7 | 4 | |
| 0.19 | 47.9 | 3.3 | 4 | | 64.3 | 2.8 | 4 | |
| 0.064 | 22.6 | 1.9 | 4 | | 38.6 | 1.0 | 4 | |
| 0.021 | 5.9 | 1.5 | 4 | | 16.5 | 2.3 | 4 | |
| 0.0071 | 1.5 | 0.4 | 4 | | 4.7 | 1.4 | 4 | |
| 0.0024 | 0.5 | 0.2 | 4 | | 0.8 | 0.3 | 4 | |
| 0.0007 | 0.3 | 0.1 | 4 | | 0.4 | 0.1 | 4 | |
| 0.0002 | 0.3 | 0.1 | 4 | | 0.2 | 0.1 | 4 | |

### Example 9. In Vivo Editing Using Two Different Guide Formats in a Humanized PCSK9 Mouse Model Using Lipid Nanoparticles (LNPs)

Selected modified guide designs from Table 2 were tested for editing efficiency in vivo. Male transgenic mice comprising a human PCSK9 gene sequence (hPCSK9) in their genomes, were used in each study involving mice. The hPCSK9 mice were generated on a hybrid C57B6/129 background, then backcrossed once to B6, and then intercrossed for cohort expansion. The hPCSK9 mice had the mouse PCSK9 gene excised out of their genomes. Animals were about 6 weeks old and were weighed pre-dose. LNPs were dosed via the lateral tail vein at 0.1, 0.3 and 1 milligrams per kilogram body weight (e.g., 0.1 mg/kg, or 0.1 mpk) respectively. The animals were observed at approximately 24 hours post dose for adverse effects. Animals were euthanized at 7 days post dose by exsanguination under isoflurane anesthesia and cervical dislocation. Blood was collected via cardiac puncture into serum separator tubes or into tubes containing buffered sodium citrate for plasma as described herein. For studies involving in vivo editing, liver tissue was collected for DNA extraction and analysis.

For the in vivo studies, genomic DNA was extracted from liver tissue using a bead-based extraction kit, e.g., the Zymo Quick-DNA 96 kit (Zymo Research, Cat. #D3010) according to the manufacturer's protocol, which includes homogenizing the tissue in lysis buffer (approximately 600 μL/10 mg tissue). All DNA samples were normalized to 100 ng/μL concentration for PCR and subsequent NGS analysis, as described in Example 1.

### Example 9.1. In Vivo Editing and Serum hPCSK9 Knockdown

Blood was collected, and the serum was isolated as described above. The total PCSK9 serum levels were determined using a human PCSK9 ELISA Kit (Abcam, Cat. ab209884). Kit reagents and standards were prepared according to the manufacturer's protocol. Mouse serum was diluted between 5 to 10-fold. Both standard curve dilutions and diluted serum samples were added to each well of the ELISA plate. An antibody cocktail containing both the capture and detection antibody was added to every well containing standard or sample. The plate was incubated at room temperature for 60 minutes with shaking before washing. Chromogenic solution was added to the plate and incubated in the dark on a shaker plate for 10 minutes before adding stop solution, e.g., sulfuric acid (approximately 0.3 M). The plate was read on a SpectraMax M5 or Clariostar plate reader at an absorbance of 450 nm. Serum hPCSK9 levels were calculated by SoftMax Pro software ver. 6.4.2 or Mars software ver. 3.31 using a four-parameter logistic curve fit off the standard curve. Final serum values were adjusted for the assay dilution. Percent protein knockdown (% KD) values were determined relative to predose levels.

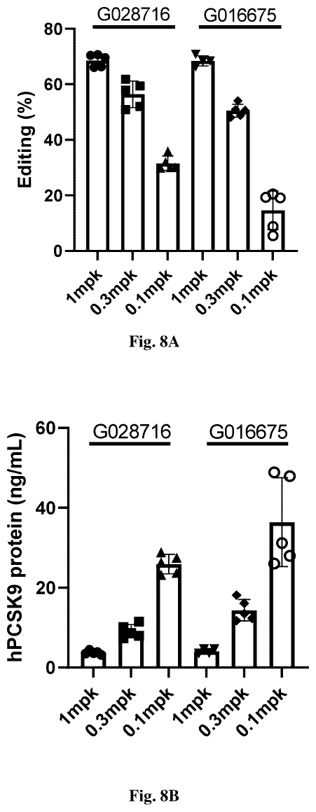

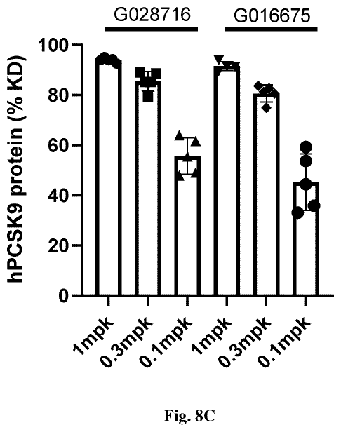

The average editing efficiency, hPCSK9 serum protein levels, and percent serum hPCSK9 knockdown (% KD) compared to predose hPCSK9 protein levels are shown in Table 22. Liver editing, hPCSK9 protein levels and percent hPCSK9 KD levels are illustrated in FIGS. 8A-8C, respectively.

TABLE 22
Editing efficiency, hPCSK9 protein levels, and % knockdown for selected guides

| | | | | | hPCSK9 | | hPCSK9 | |
|---|---|---|---|---|---|---|---|---|
| | | Dose | Editing | | protein | | protein (% | |
| Guide | n | (mpk) | (%) | SD | (ng/ml) | SD | KD) | SD |
| G028716 | 5 | 1 | 68.6 | 2.2 | 3.7 | 0.5 | 94.0 | 0.9 |
| G028716 | 5 | 0.3 | 56.4 | 4.7 | 9.1 | 1.7 | 85.5 | 3.9 |
| G028716 | 5 | 0.1 | 31.6 | 2.6 | 25.9 | 2.4 | 55.7 | 7.2 |
| G016675 | 4 | 1 | 68.5 | 1.8 | 4.1 | 0.6 | 91.6 | 1.8 |
| G016675 | 5 | 0.3 | 50.5 | 2.3 | 14.3 | 2.7 | 80.7 | 3.4 |
| G016675 | 5 | 0.1 | 14.6 | 7.0 | 36.4 | 11.1 | 45.3 | 11.3 |

In the following table of sequences, the terms “mA,” “mC,” “mU,” or “mG” are used to denote a nucleotide that has been modified with 2′-O-Me. In the following table, each “N” is used to independently denote any nucleotide (e.g., A, U, T, C, G). In certain embodiments, the nucleotide is an unmodified RNA nucleotide residue, i.e., a ribose sugar and a phosphodiester backbone. In the following table, a “*” is used to denote a PS modification. In this application, the terms A*, C*, U*, or G* may be used to denote a nucleotide that is linked to the next (e.g., 3′) nucleotide with a PS bond. It is understood that if a DNA sequence (comprising Ts) is referenced with respect to an RNA, then Ts should be replaced with Us (which may be modified or unmodified depending on the context), and vice versa. In the following table, single amino acid letter code is used to provide peptide sequences.

TABLE 23
Additional Sequences

| SEQ | | |
|---|---|---|
| ID | | |
| NO | Description | Sequence |
| 1001 | Spy Cas9 amino | MDKKYSIGLDIGTNSVGWAVITDEYKVPSKKFKVLGNTDRHSIKKNLIGALLFDS |
| | acid sequence | GETAEATRLKRTARRRYTRRKNRICYLQEIFSNEMAKVDDSFFHRLEESFLVEED |
| | with 1x NLS | KKHERHPIFGNIVDEVAYHEKYPTIYHLRKKLVDSTDKADLRLIYLALAHMIKER |
| | (RNP) | GHFLIEGDLNPDNSDVDKLFIQLVQTYNQLFEENPINASGVDAKAILSARLSKSR |
| | | RLENLIAQLPGEKKNGLFGNLIALSLGLTPNEKSNEDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPFLKDNREKI |
| | | EKILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRENASLGTYHDLLKIIKDKD |
| | | ELDNEENEDILEDIVLTLTLFEDREMIEERLKTYAHLFDDKVMKQLKRRRYTGWG |
| | | RLSRKLINGIRDKQSGKTILDELKSDGFANRNEMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKEDNLTKAERGGLSELDKAGFIKRQLVETRQITKHVAQIL |
| | | DSRMNTKYDENDKLIREVKVITLKSKLVSDFRKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGFSKESILPKRNSDKLIARKKDWDPKKYGGFDSPTVAYSVLVVAKVEKGKSK |
| | | KLKSVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKK |
| | | KRKV |
| 1002 | mRNA encoding | GGGAAGCUCAGAAUAAACGCUCAACUUUGGCCGGAUCUGCCACCAUGGACAAGAA |
| | Spy Cas9 | GUACUCCAUCGGCCUGGACAUCGGCACCAACUCCGUGGGCUGGGCCGUGAUCACC |
| | | GACGAGUACAAGGUGCCCUCCAAGAAGUUCAAGGUGCUGGGCAACACCGACCGGC |
| | | ACUCCAUCAAGAAGAACCUGAUCGGCGCCCUGCUGUUCGACUCCGGCGAGACCGC |
| | | CGAGGCCACCCGGCUGAAGCGGACCGCCCGGCGGCGGUACACCCGGCUUCCUGGU |
| | | GGAGGAGGACAAGAAGCACGAGCGGCACCCCAUCUUCGGCAACAUCGUGGACGAG |
| | | GUGGCCUACCACGAGAAGUACCCCACCAUCUACCACCUGCGGAAGAAGCUGGUGG |
| | | ACUCCACCGACAAGGCCGACCUGCGGCUGAUCUACCUGGCCCUGGCCCACAUGAU |
| | | CAAGUUCCGGGGCCACUUCCUGAUCGAGGGCGACCUGAACCCCGACAACUCCGAC |
| | | GUGGACAAGCUGUUCAUCCAGCUGGUGCAGACCUACAACCAGCUGUUCGAGGAGA |
| | | ACCCCAUCAACGCCUCCGGCGUGGACGCCAAGGCCAUCCUGUCCGCCCGGCUGUC |
| | | CAAGUCCCGGCGGCUGGAGAACCUGAUCGCCCAGCUGCCCGGCGAGAAGAAGAAC |
| | | GGCCUGUUCGGCAACCUGAUCGCCCUGUCCCUGGGCCUGACCCCCAACUUCAAGU |
| | | CCAACUUCGACCUGGCCGAGGACGCCAAGCUGCAGCUGUCCAAGGACACCUACGA |
| | | CGACGACCUGGACAACCUGCUGGCCCAGAUCGGCGACCAGUACGCCGACCUGUUC |
| | | CUGGCCGCCAAGAACCUGUCCGACGCCAUCCUGCUGUCCGACAUCCUGCGGGUGA |
| | | ACACCGAGAUCACCAAGGCCCCCCUGUCCGCCUCCAUGAUCAAGCGGUACGACGA |
| | | GCACCACCAGGACCUGACCCUGCUGAAGGCCCUGGUGCGGCAGCAGCUGCCCGAG |
| | | AAGUACAAGGAGAUCUUCUUCGACCAGUCCAAGAACGGCUACGCCGGCUACAUCG |
| | | ACGGCGGCGCCUCCCAGGAGGAGUUCUACAAGUUCAUCAAGCCCAUCCUGGAGAA |
| | | GAUGGACGGCACCGAGGAGCUGCUGGUGAAGCUGAACCGGGAGGACCUGCUGCGG |
| | | AAGCAGCGGACCUUCGACAACGGCUCCAUCCCCCACCAGAUCCACCUGGGCGAGC |
| | | UGCACGCCAUCCUGCGGCGGCAGGAGGACUUCUACCCCUUCCUGAAGGACAACCG |
| | | GGAGAAGAUCGAGAAGAUCCUGACCUUCCGGAUCCCCUACUACGUGGGCCCCCUG |
| | | GCCCGGGGCAACUCCCGGUUCGCCUGGAUGACCCGGAAGUCCGAGGAGACCAUCA |
| | | CCCCCUGGAACUUCGAGGAGGUGGUGGACAAGGGCGCCUCCGCCCAGUCCUUCAU |
| | | CGAGCGGAUGACCAACUUCGACAAGAACCUGCCCAACGAGAAGGUGCUGCCCAAG |
| | | CACUCCCUGCUGUACGAGUACUUCACCGUGUACAACGAGCUGACCAAGGUGAAGU |
| | | ACGUGACCGAGGGCAUGCGGAAGCCCGCCUUCCUGUCCGGCGAGCAGAAGAAGGC |
| | | CAUCGUGGACCUGCUGUUCAAGACCAACCGGAAGGUGACCGUGAAGCAGCUGAAG |
| | | GAGGACUACUUCAAGAAGAUCGAGUGCUUCGACUCCGUGGAGAUCUCCGGCGUGG |
| | | AGGACCGGUUCAACGCCUCCCUGGGCACCUACCACGACCUGCUGAAGAUCAUCAA |
| | | GGACAAGGACUUCCUGGACAACGAGGAGAACGAGGACAUCCUGGAGGACAUCGUG |
| | | CUGACCCUGACCCUGUUCGAGGACCGGGAGAUGAUCGAGGAGCGGCUGAAGACCU |
| | | ACGCCCACCUGUUCGACGACAAGGUGAUGAAGCAGCUGAAGCGGCGGCGGUACAC |
| | | CGGCUGGGGCCGGCUGUCCCGGAAGCUGAUCAACGGCAUCCGGGACAAGCAGUCC |
| | | GGCAAGACCAUCCUGGACUUCCUGAAGUCCGACGGCUUCGCCAACCGGAACUUCA |
| | | UGCAGCUGAUCCACGACGACUCCCUGACCUUCAAGGAGGACAUCCAGAAGGCCCA |
| | | GGUGUCCGGCCAGGGCGACUCCCUGCACGAGCACAUCGCCAACCUGGCCGGCUCC |
| | | CCCGCCAUCAAGAAGGGCAUCCUGCAGACCGUGAAGGUGGUGGACGAGCUGGUGA |
| | | AGGUGAUGGGCCGGCACAAGCCCGAGAACAUCGUGAUCGAGAUGGCCCGGGAGAA |
| | | CCAGACCACCCAGAAGGGCCAGAAGAACUCCCGGGAGCGGAUGAAGCGGAUCGAG |
| | | GAGGGCAUCAAGGAGCUGGGCUCCCAGAUCCUGAAGGAGCACCCCGUGGAGAACA |
| | | CCCAGCUGCAGAACGAGAAGCUGUACCUGUACUACCUGCAGAACGGCCGGGACAU |
| | | GUACGUGGACCAGGAGCUGGACAUCAACCGGCUGUCCGACUACGACGUGGACCAC |
| | | AUCGUGCCCCAGUCCUUCCUGAAGGACGACUCCAUCGACAACAAGGUGCUGACCC |
| | | GGUCCGACAAGAACCGGGGCAAGUCCGACAACGUGCCCUCCGAGGAGGUGGUGAA |
| | | GAAGAUGAAGAACUACUGGCGGCAGCUGCUGAACGCCAAGCUGAUCACCCAGCGG |
| | | AAGUUCGACAACCUGACCAAGGCCGAGCGGGGCGGCCUGUCCGAGCUGGACAAGG |
| | | CCGGCUUCAUCAAGCGGCAGCUGGUGGAGACCCGGCAGAUCACCAAGCACGUGGC |
| | | CCAGAUCCUGGACUCCCGGAUGAACACCAAGUACGACGAGAACGACAAGCUGAUC |
| | | CGGGAGGUGAAGGUGAUCACCCUGAAGUCCAAGCUGGUGUCCGACUUCCGGAAGG |
| | | ACUUCCAGUUCUACAAGGUGCGGGAGAUCAACAACUACCACCACGCCCACGACGC |
| | | CUACCUGAACGCCGUGGUGGGCACCGCCCUGAUCAAGAAGUACCCCAAGCUGGAG |
| | | UCCGAGUUCGUGUACGGCGACUACAAGGUGUACGACGUGCGGAAGAUGAUCGCCA |
| | | AGUCCGAGCAGGAGAUCGGCAAGGCCACCGCCAAGUACUUCUUCUACUCCAACAU |
| | | CAUGAACUUCUUCAAGACCGAGAUCACCCUGGCCAACGGCGAGAUCCGGAAGCGG |
| | | CCCCUGAUCGAGACCAACGGCGAGACCGGCGAGAUCGUGUGGGACAAGGGCCGGG |
| | | ACUUCGCCACCGUGCGGAAGGUGCUGUCCAUGCCCCAGGUGAACAUCGUGAAGAA |
| | | GACCGAGGUGCAGACCGGCGGCUUCUCCAAGGAGUCCAUCCUGCCCAAGCGGAAC |
| | | UCCGACAAGCUGAUCGCCCGGAAGAAGGACUGGGACCCCAAGAAGUACGGCGGCU |
| | | UCGACUCCCCCACCGUGGCCUACUCCGUGCUGGUGGUGGCCAAGGUGGAGAAGGG |
| | | CAAGUCCAAGAAGCUGAAGUCCGUGAAGGAGCUGCUGGGCAUCACCAUCAUGGAG |
| | | CGGUCCUCCUUCGAGAAGAACCCCAUCGACUUCCUGGAGGCCAAGGGCUACAAGG |
| | | AGGUGAAGAAGGACCUGAUCAUCAAGCUGCCCAAGUACUCCCUGUUCGAGCUGGA |
| | | GAACGGCCGGAAGCGGAUGCUGGCCUCCGCCGGCGAGCUGCAGAAGGGCAACGAG |
| | | CUGGCCCUGCCCUCCAAGUACGUGAACUUCCUGUACCUGGCCUCCCACUACGAGA |
| | | AGCUGAAGGGCUCCCCCGAGGACAACGAGCAGAAGCAGCUGUUCGUGGAGCAGCA |
| | | CAAGCACUACCUGGACGAGAUCAUCGAGCAGAUCUCCGAGUUCUCCAAGCGGGUG |
| | | AUCCUGGCCGACGCCAACCUGGACAAGGUGCUGUCCGCCUACAACAAGCACCGGG |
| | | ACAAGCCCAUCCGGGAGCAGGCCGAGAACAUCAUCCACCUGUUCACCCUGACCAA |
| | | CCUGGGCGCCCCCGCCGCCUUCAAGUACUUCGACACCACCAUCGACCGGAAGCGG |
| | | UACACCUCCACCAAGGAGGUGCUGGACGCCACCCUGAUCCACCAGUCCAUCACCG |
| | | GCCUGUACGAGACCCGGAUCGACCUGUCCCAGCUGGGCGGCGACGGCGGCGGCUC |
| | | CCCCAAGAAGAAGCGGAAGGUGUGACUAGCACCAGCCUCAAGAACACCCGAAUGG |
| | | AGUCUCUAAGCUACAUAAUACCAACUUACACUUUACAAAAUGUUGUCCCCCAAAA |
| | | UGUAGCCAUUCGUAUCUGCUCCUAAUAAAAAGAAAGUUUCUUCACAUUCUCUCGA |
| | | GAAAAAAAAAAAAAAAAAAAAAAAAAAAAAAAAAAAAAAAAAAAAAAAAAAAAAA |
| | | AAAAAAAAAAAAAAAAAAAAAAAAAAAAAAAAAAAAAAAUCUAG |
| 1003 | Open reading | auggacaagaaguacuccaucggccuggacaucggcaccaacuccgugggcuggg |
| | frame for Spy | ccgugaucaccgacgaguacaaggugcccuccaagaaguucaaggugcugggcaa |
| | Cas9 | caccgaccggcacuccaucaagaagaaccugaucggcgcccugcuguucgacucc |
| | | ggcgagaccgccgaggccacccggcugaagcggaccgcccggcggcgguacaccc |
| | | ggcggaagaaccggaucugcuaccugcaggagaucuucuccaacgagauggccaa |
| | | gguggacgacuccuucuuccaccggcuggaggaguccuuccugguggaggaggac |
| | | aagaagcacgagcggcaccccaucuucggcaacaucguggacgagguggccuacc |
| | | acgagaaguaccccaccaucuaccaccugcggaagaagcugguggacuccaccga |
| | | caaggccgaccugcggcugaucuaccuggcccuggcccacaugaucaaguuccgg |
| | | ggccacuuccugaucgagggcgaccugaaccccgacaacuccgacguggacaagc |
| | | uguucauccagcuggugcagaccuacaaccagcuguucgaggagaaccccaucaa |
| | | cgccuccggcguggacgccaaggccauccuguccgcccggcuguccaagucccgg |
| | | cggcuggagaaccugaucgcccagcugcccggcgagaagaagaacggccuguucg |
| | | gcaaccugaucgcccugucccugggccugacccccaacuucaaguccaacuucga |
| | | ccuggccgaggacgccaagcugcagcuguccaaggacaccuacgacgacgaccug |
| | | gacaaccugcuggcccagaucggcgaccaguacgccgaccuguuccuggccgcca |
| | | agaaccuguccgacgccauccugcuguccgacauccugcgggugaacaccgagau |
| | | caccaaggccccccuguccgccuccaugaucaagcgguacgacgagcaccaccag |
| | | gaccugacccugcugaaggcccuggugcggcagcagcugcccgagaaguacaagg |
| | | agaucuucuucgaccaguccaagaacggcuacgccggcuacaucgacggcggcgc |
| | | cucccaggaggaguucuacaaguucaucaagcccauccuggagaagauggacggc |
| | | accgaggagcugcuggugaagcugaaccgggaggaccugcugcggaagcagcgga |
| | | ccuucgacaacggcuccaucccccaccagauccaccugggcgagcugcacgccau |
| | | ccugcggcggcaggaggacuucuaccccuuccugaaggacaaccgggagaagauc |
| | | gagaagauccugaccuuccggauccccuacuacgugggcccccuggcccggggca |
| | | acucccgguucgccuggaugacccggaaguccgaggagaccaucacccccuggaa |
| | | cuucgaggaggugguggacaagggcgccuccgcccaguccuucaucgagcggaug |
| | | accaacuucgacaagaaccugcccaacgagaaggugcugcccaagcacucccugc |
| | | uguacgaguacuucaccguguacaacgagcugaccaaggugaaguacgugaccga |
| | | gggcaugcggaagcccgccuuccuguccggcgagcagaagaaggccaucguggac |
| | | cugcuguucaagaccaaccggaaggugaccgugaagcagcugaaggaggacuacu |
| | | ucaagaagaucgagugcuucgacuccguggagaucuccggcguggaggaccgguu |
| | | caacgccucccugggcaccuaccacgaccugcugaagaucaucaaggacaaggac |
| | | uuccuggacaacgaggagaacgaggacauccuggaggacaucgugcugacccuga |
| | | cccuguucgaggaccgggagaugaucgaggagcggcugaagaccuacgcccaccu |
| | | guucgacgacaaggugaugaagcagcugaagcggcggcgguacaccggcuggggc |
| | | cggcugucccggaagcugaucaacggcauccgggacaagcaguccggcaagacca |
| | | uccuggacuuccugaaguccgacggcuucgccaaccggaacuucaugcagcugau |
| | | ccacgacgacucccugaccuucaaggaggacauccagaaggcccagguguccggc |
| | | cagggcgacucccugcacgagcacaucgccaaccuggccggcucccccgccauca |
| | | agaagggcauccugcagaccgugaaggugguggacgagcuggugaaggugauggg |
| | | ccggcacaagcccgagaacaucgugaucgagauggcccgggagaaccagaccacc |
| | | cagaagggccagaagaacucccgggagcggaugaagcggaucgaggagggcauca |
| | | aggagcugggcucccagauccugaaggagcaccccguggagaacacccagcugca |
| | | gaacgagaagcuguaccuguacuaccugcagaacggccgggacauguacguggac |
| | | caggagcuggacaucaaccggcuguccgacuacgacguggaccacaucgugcccc |
| | | aguccuuccugaaggacgacuccaucgacaacaaggugcugacccgguccgacaa |
| | | gaaccggggcaaguccgacaacgugcccuccgaggagguggugaagaagaugaag |
| | | aacuacuggcggcagcugcugaacgccaagcugaucacccagcggaaguucgaca |
| | | accugaccaaggccgagcggggcggccuguccgagcuggacaaggccggcuucau |
| | | caagcggcagcugguggagacccggcagaucaccaagcacguggcccagauccug |
| | | gacucccggaugaacaccaaguacgacgagaacgacaagcugauccgggagguga |
| | | aggugaucacccugaaguccaagcugguguccgacuuccggaaggacuuccaguu |
| | | cuacaaggugcgggagaucaacaacuaccaccacgcccacgacgccuaccugaac |
| | | gccguggugggcaccgcccugaucaagaaguaccccaagcuggaguccgaguucg |
| | | uguacggcgacuacaagguguacgacgugcggaagaugaucgccaaguccgagca |
| | | ggagaucggcaaggccaccgccaaguacuucuucuacuccaacaucaugaacuuc |
| | | uucaagaccgagaucacccuggccaacggcgagauccggaagcggccccugaucg |
| | | agaccaacggcgagaccggcgagaucgugugggacaagggccgggacuucgccac |
| | | cgugcggaaggugcuguccaugccccaggugaacaucgugaagaagaccgaggug |
| | | cagaccggcggcuucuccaaggaguccauccugcccaagcggaacuccgacaagc |
| | | ugaucgcccggaagaaggacugggaccccaagaaguacggcggcuucgacucccc |
| | | caccguggccuacuccgugcuggugguggccaagguggagaagggcaaguccaag |
| | | aagcugaaguccgugaaggagcugcugggcaucaccaucauggagcgguccuccu |
| | | ucgagaagaaccccaucgacuuccuggaggccaagggcuacaaggaggugaagaa |
| | | ggaccugaucaucaagcugcccaaguacucccuguucgagcuggagaacggccgg |
| | | aagcggaugcuggccuccgccggcgagcugcagaagggcaacgagcuggcccugc |
| | | ccuccaaguacgugaacuuccuguaccuggccucccacuacgagaagcugaaggg |
| | | cucccccgaggacaacgagcagaagcagcuguucguggagcagcacaagcacuac |
| | | cuggacgagaucaucgagcagaucuccgaguucuccaagcgggugauccuggccg |
| | | acgccaaccuggacaaggugcuguccgccuacaacaagcaccgggacaagcccau |
| | | ccgggagcaggccgagaacaucauccaccuguucacccugaccaaccugggcgcc |
| | | cccgccgccuucaaguacuucgacaccaccaucgaccggaagcgguacaccucca |
| | | ccaaggaggugcuggacgccacccugauccaccaguccaucaccggccuguacga |
| | | gacccggaucgaccugucccagcugggcggcgacggcggcggcucccccaagaag |
| | | aagcggaagguguga |
| 1004 | Amino acid | MDKKYSIGLDIGTNSVGWAVITDEYKVPSKKFKVLGNTDRHSIKKNLIGALLEDS |
| | sequence for | GETAEATRLKRTARRRYTRRKNRICYLQEIFSNEMAKVDDSFFHRLEESFLVEED |
| | Spy Cas9 | KKHERHPIFGNIVDEVAYHEKYPTIYHLRKKLVDSTDKADLRLIYLALAHMIKER |
| | | GHFLIEGDLNPDNSDVDKLFIQLVQTYNQLFEENPINASGVDAKAILSARLSKSR |
| | | RLENLIAQLPGEKKNGLFGNLIALSLGLTPNEKSNEDLAEDAKLQLSKDTYDDDL |
| | | DNLLAQIGDQYADLELAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPELKDNREKI |
| | | EKILTFRIPYYVGPLARGNSRFAWMTRKSEETITPWNFEEVVDKGASAQSFIERM |
| | | TNEDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRENASLGTYHDLLKIIKDKD |
| | | ELDNEENEDILEDIVLTLTLFEDREMIEERLKTYAHLEDDKVMKQLKRRRYTGWG |
| | | RLSRKLINGIRDKQSGKTILDELKSDGFANRNEMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQIL |
| | | DSRMNTKYDENDKLIREVKVITLKSKLVSDERKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGFSKESILPKRNSDKLIARKKDWDPKKYGGFDSPTVAYSVLVVAKVEKGKSK |
| | | KLKSVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKK |
| | | KRKV* |
| 1005 | mRNA encoding | GGGUCCCGCAGUCGGCGUCCAGCGGCUCUGCUUGUUCGUGUGUGUGUCGUUGCAG |
| | Spy Cas9 | GCCUUAUUCGGAUCCGCCACCAUGGACAAGAAGUACAGCAUCGGACUGGACAUCG |
| | | GAACAAACAGCGUCGGAUGGGCAGUCAUCACAGACGAAUACAAGGUCCCGAGCAA |
| | | GAAGUUCAAGGUCCUGGGAAACACAGACAGACACAGCAUCAAGAAGAACCUGAUC |
| | | GGAGCACUGCUGUUCGACAGCGGAGAAACAGCAGAAGCAACAAGACUGAAGAGAA |
| | | CAGCAAGAAGAAGAUACACAAGAAGAAAGAACAGAAUCUGCUACCUGCAGGAAAU |
| | | CUUCAGCAACGAAAUGGCAAAGGUCGACGACAGCUUCUUCCACAGACUGGAAGAA |
| | | AGCUUCCUGGUCGAAGAAGACAAGAAGCACGAAAGACACCCGAUCUUCGGAAACA |
| | | UCGUCGACGAAGUCGCAUACCACGAAAAGUACCCGACAAUCUACCACCUGAGAAA |
| | | GAAGCUGGUCGACAGCACAGACAAGGCAGACCUGAGACUGAUCUACCUGGCACUG |
| | | GCACACAUGAUCAAGUUCAGAGGACACUUCCUGAUCGAAGGAGACCUGAACCCGG |
| | | ACAACAGCGACGUCGACAAGCUGUUCAUCCAGCUGGUCCAGACAUACAACCAGCU |
| | | GUUCGAAGAAAACCCGAUCAACGCAAGCGGAGUCGACGCAAAGGCAAUCCUGAGC |
| | | GCAAGACUGAGCAAGAGCAGAAGACUGGAAAACCUGAUCGCACAGCUGCCGGGAG |
| | | AAAAGAAGAACGGACUGUUCGGAAACCUGAUCGCACUGAGCCUGGGACUGACACC |
| | | GAACUUCAAGAGCAACUUCGACCUGGCAGAAGACGCAAAGCUGCAGCUGAGCAAG |
| | | GACACAUACGACGACGACCUGGACAACCUGCUGGCACAGAUCGGAGACCAGUACG |
| | | CAGACCUGUUCCUGGCAGCAAAGAACCUGAGCGACGCAAUCCUGCUGAGCGACAU |
| | | CCUGAGAGUCAACACAGAAAUCACAAAGGCACCGCUGAGCGCAAGCAUGAUCAAG |
| | | AGAUACGACGAACACCACCAGGACCUGACACUGCUGAAGGCACUGGUCAGACAGC |
| | | AGCUGCCGGAAAAGUACAAGGAAAUCUUCUUCGACCAGAGCAAGAACGGAUACGC |
| | | AGGAUACAUCGACGGAGGAGCAAGCCAGGAAGAAUUCUACAAGUUCAUCAAGCCG |
| | | AUCCUGGAAAAGAUGGACGGAACAGAAGAACUGCUGGUCAAGCUGAACAGAGAAG |
| | | ACCUGCUGAGAAAGCAGAGAACAUUCGACAACGGAAGCAUCCCGCACCAGAUCCA |
| | | CCUGGGAGAACUGCACGCAAUCCUGAGAAGACAGGAAGACUUCUACCCGUUCCUG |
| | | AAGGACAACAGAGAAAAGAUCGAAAAGAUCCUGACAUUCAGAAUCCCGUACUACG |
| | | UCGGACCGCUGGCAAGAGGAAACAGCAGAUUCGCAUGGAUGACAAGAAAGAGCGA |
| | | AGAAACAAUCACACCGUGGAACUUCGAAGAAGUCGUCGACAAGGGAGCAAGCGCA |
| | | CAGAGCUUCAUCGAAAGAAUGACAAACUUCGACAAGAACCUGCCGAACGAAAAGG |
| | | UCCUGCCGAAGCACAGCCUGCUGUACGAAUACUUCACAGUCUACAACGAACUGAC |
| | | AAAGGUCAAGUACGUCACAGAAGGAAUGAGAAAGCCGGCAUUCCUGAGCGGAGAA |
| | | CAGAAGAAGGCAAUCGUCGACCUGCUGUUCAAGACAAACAGAAAGGUCACAGUCA |
| | | AGCAGCUGAAGGAAGACUACUUCAAGAAGAUCGAAUGCUUCGACAGCGUCGAAAU |
| | | CAGCGGAGUCGAAGACAGAUUCAACGCAAGCCUGGGAACAUACCACGACCUGCUG |
| | | AAGAUCAUCAAGGACAAGGACUUCCUGGACAACGAAGAAAACGAAGACAUCCUGG |
| | | AAGACAUCGUCCUGACACUGACACUGUUCGAAGACAGAGAAAUGAUCGAAGAAAG |
| | | ACUGAAGACAUACGCACACCUGUUCGACGACAAGGUCAUGAAGCAGCUGAAGAGA |
| | | AGAAGAUACACAGGAUGGGGAAGACUGAGCAGAAAGCUGAUCAACGGAAUCAGAG |
| | | ACAAGCAGAGCGGAAAGACAAUCCUGGACUUCCUGAAGAGCGACGGAUUCGCAAA |
| | | CAGAAACUUCAUGCAGCUGAUCCACGACGACAGCCUGACAUUCAAGGAAGACAUC |
| | | CAGAAGGCACAGGUCAGCGGACAGGGAGACAGCCUGCACGAACACAUCGCAAACC |
| | | UGGCAGGAAGCCCGGCAAUCAAGAAGGGAAUCCUGCAGACAGUCAAGGUCGUCGA |
| | | CGAACUGGUCAAGGUCAUGGGAAGACACAAGCCGGAAAACAUCGUCAUCGAAAUG |
| | | GCAAGAGAAAACCAGACAACACAGAAGGGACAGAAGAACAGCAGAGAAAGAAUGA |
| | | AGAGAAUCGAAGAAGGAAUCAAGGAACUGGGAAGCCAGAUCCUGAAGGAACACCC |
| | | GGUCGAAAACACACAGCUGCAGAACGAAAAGCUGUACCUGUACUACCUGCAGAAC |
| | | GGAAGAGACAUGUACGUCGACCAGGAACUGGACAUCAACAGACUGAGCGACUACG |
| | | ACGUCGACCACAUCGUCCCGCAGAGCUUCCUGAAGGACGACAGCAUCGACAACAA |
| | | GGUCCUGACAAGAAGCGACAAGAACAGAGGAAAGAGCGACAACGUCCCGAGCGAA |
| | | GAAGUCGUCAAGAAGAUGAAGAACUACUGGAGACAGCUGCUGAACGCAAAGCUGA |
| | | UCACACAGAGAAAGUUCGACAACCUGACAAAGGCAGAGAGAGGAGGACUGAGCGA |
| | | ACUGGACAAGGCAGGAUUCAUCAAGAGACAGCUGGUCGAAACAAGACAGAUCACA |
| | | AAGCACGUCGCACAGAUCCUGGACAGCAGAAUGAACACAAAGUACGACGAAAACG |
| | | ACAAGCUGAUCAGAGAAGUCAAGGUCAUCACACUGAAGAGCAAGCUGGUCAGCGA |
| | | CUUCAGAAAGGACUUCCAGUUCUACAAGGUCAGAGAAAUCAACAACUACCACCAC |
| | | GCACACGACGCAUACCUGAACGCAGUCGUCGGAACAGCACUGAUCAAGAAGUACC |
| | | CGAAGCUGGAAAGCGAAUUCGUCUACGGAGACUACAAGGUCUACGACGUCAGAAA |
| | | GAUGAUCGCAAAGAGCGAACAGGAAAUCGGAAAGGCAACAGCAAAGUACUUCUUC |
| | | UACAGCAACAUCAUGAACUUCUUCAAGACAGAAAUCACACUGGCAAACGGAGAAA |
| | | UCAGAAAGAGACCGCUGAUCGAAACAAACGGAGAAACAGGAGAAAUCGUCUGGGA |
| | | CAAGGGAAGAGACUUCGCAACAGUCAGAAAGGUCCUGAGCAUGCCGCAGGUCAAC |
| | | AUCGUCAAGAAGACAGAAGUCCAGACAGGAGGAUUCAGCAAGGAAAGCAUCCUGC |
| | | CGAAGAGAAACAGCGACAAGCUGAUCGCAAGAAAGAAGGACUGGGACCCGAAGAA |
| | | GUACGGAGGAUUCGACAGCCCGACAGUCGCAUACAGCGUCCUGGUCGUCGCAAAG |
| | | GUCGAAAAGGGAAAGAGCAAGAAGCUGAAGAGCGUCAAGGAACUGCUGGGAAUCA |
| | | CAAUCAUGGAAAGAAGCAGCUUCGAAAAGAACCCGAUCGACUUCCUGGAAGCAAA |
| | | GGGAUACAAGGAAGUCAAGAAGGACCUGAUCAUCAAGCUGCCGAAGUACAGCCUG |
| | | UUCGAACUGGAAAACGGAAGAAAGAGAAUGCUGGCAAGCGCAGGAGAACUGCAGA |
| | | AGGGAAACGAACUGGCACUGCCGAGCAAGUACGUCAACUUCCUGUACCUGGCAAG |
| | | CCACUACGAAAAGCUGAAGGGAAGCCCGGAAGACAACGAACAGAAGCAGCUGUUC |
| | | GUCGAACAGCACAAGCACUACCUGGACGAAAUCAUCGAACAGAUCAGCGAAUUCA |
| | | GCAAGAGAGUCAUCCUGGCAGACGCAAACCUGGACAAGGUCCUGAGCGCAUACAA |
| | | CAAGCACAGAGACAAGCCGAUCAGAGAACAGGCAGAAAACAUCAUCCACCUGUUC |
| | | ACACUGACAAACCUGGGAGCACCGGCAGCAUUCAAGUACUUCGACACAACAAUCG |
| | | ACAGAAAGAGAUACACAAGCACAAAGGAAGUCCUGGACGCAACACUGAUCCACCA |
| | | GAGCAUCACAGGACUGUACGAAACAAGAAUCGACCUGAGCCAGCUGGGAGGAGAC |
| | | GGAGGAGGAAGCCCGAAGAAGAAGAGAAAGGUCUAGCUAGCCAUCACAUUUAAAA |
| | | GCAUCUCAGCCUACCAUGAGAAUAAGAGAAAGAAAAUGAAGAUCAAUAGCUUAUU |
| | | CAUCUCUUUUUCUUUUUCGUUGGUGUAAAGCCAACACCCUGUCUAAAAAACAUAA |
| | | AUUUCUUUAAUCAUUUUGCCUCUUUUCUCUGUGCUUCAAUUAAUAAAAAAUGGAA |
| | | AGAACCUCGAGAAAAAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAAAAAA |
| | | AUCUAG |
| 1006 | Open reading | AUGGACAAGAAGUACAGCAUCGGACUGGACAUCGGAACAAACAGCGUCGGAUGGG |
| | frame for Spy | CAGUCAUCACAGACGAAUACAAGGUCCCGAGCAAGAAGUUCAAGGUCCUGGGAAA |
| | Cas9 | CACAGACAGACACAGCAUCAAGAAGAACCUGAUCGGAGCACUGCUGUUCGACAGC |
| | | GGAGAAACAGCAGAAGCAACAAGACUGAAGAGAACAGCAAGAAGAAGAUACACAA |
| | | GAAGAAAGAACAGAAUCUGCUACCUGCAGGAAAUCUUCAGCAACGAAAUGGCAAA |
| | | GGUCGACGACAGCUUCUUCCACAGACUGGAAGAAAGCUUCCUGGUCGAAGAAGAC |
| | | AAGAAGCACGAAAGACACCCGAUCUUCGGAAACAUCGUCGACGAAGUCGCAUACC |
| | | ACGAAAAGUACCCGACAAUCUACCACCUGAGAAAGAAGCUGGUCGACAGCACAGA |
| | | CAAGGCAGACCUGAGACUGAUCUACCUGGCACUGGCACACAUGAUCAAGUUCAGA |
| | | GGACACUUCCUGAUCGAAGGAGACCUGAACCCGGACAACAGCGACGUCGACAAGC |
| | | UGUUCAUCCAGCUGGUCCAGACAUACAACCAGCUGUUCGAAGAAAACCCGAUCAA |
| | | CGCAAGCGGAGUCGACGCAAAGGCAAUCCUGAGCGCAAGACUGAGCAAGAGCAGA |
| | | AGACUGGAAAACCUGAUCGCACAGCUGCCGGGAGAAAAGAAGAACGGACUGUUCG |
| | | GAAACCUGAUCGCACUGAGCCUGGGACUGACACCGAACUUCAAGAGCAACUUCGA |
| | | CCUGGCAGAAGACGCAAAGCUGCAGCUGAGCAAGGACACAUACGACGACGACCUG |
| | | GACAACCUGCUGGCACAGAUCGGAGACCAGUACGCAGACCUGUUCCUGGCAGCAA |
| | | AGAACCUGAGCGACGCAAUCCUGCUGAGCGACAUCCUGAGAGUCAACACAGAAAU |
| | | CACAAAGGCACCGCUGAGCGCAAGCAUGAUCAAGAGAUACGACGAACACCACCAG |
| | | GACCUGACACUGCUGAAGGCACUGGUCAGACAGCAGCUGCCGGAAAAGUACAAGG |
| | | AAAUCUUCUUCGACCAGAGCAAGAACGGAUACGCAGGAUACAUCGACGGAGGAGC |
| | | AAGCCAGGAAGAAUUCUACAAGUUCAUCAAGCCGAUCCUGGAAAAGAUGGACGGA |
| | | ACAGAAGAACUGCUGGUCAAGCUGAACAGAGAAGACCUGCUGAGAAAGCAGAGAA |
| | | CAUUCGACAACGGAAGCAUCCCGCACCAGAUCCACCUGGGAGAACUGCACGCAAU |
| | | CCUGAGAAGACAGGAAGACUUCUACCCGUUCCUGAAGGACAACAGAGAAAAGAUC |
| | | GAAAAGAUCCUGACAUUCAGAAUCCCGUACUACGUCGGACCGCUGGCAAGAGGAA |
| | | ACAGCAGAUUCGCAUGGAUGACAAGAAAGAGCGAAGAAACAAUCACACCGUGGAA |
| | | CUUCGAAGAAGUCGUCGACAAGGGAGCAAGCGCACAGAGCUUCAUCGAAAGAAUG |
| | | ACAAACUUCGACAAGAACCUGCCGAACGAAAAGGUCCUGCCGAAGCACAGCCUGC |
| | | UGUACGAAUACUUCACAGUCUACAACGAACUGACAAAGGUCAAGUACGUCACAGA |
| | | AGGAAUGAGAAAGCCGGCAUUCCUGAGCGGAGAACAGAAGAAGGCAAUCGUCGAC |
| | | CUGCUGUUCAAGACAAACAGAAAGGUCACAGUCAAGCAGCUGAAGGAAGACUACU |
| | | UCAAGAAGAUCGAAUGCUUCGACAGCGUCGAAAUCAGCGGAGUCGAAGACAGAUU |
| | | CAACGCAAGCCUGGGAACAUACCACGACCUGCUGAAGAUCAUCAAGGACAAGGAC |
| | | UUCCUGGACAACGAAGAAAACGAAGACAUCCUGGAAGACAUCGUCCUGACACUGA |
| | | CACUGUUCGAAGACAGAGAAAUGAUCGAAGAAAGACUGAAGACAUACGCACACCU |
| | | GUUCGACGACAAGGUCAUGAAGCAGCUGAAGAGAAGAAGAUACACAGGAUGGGGA |
| | | AGACUGAGCAGAAAGCUGAUCAACGGAAUCAGAGACAAGCAGAGCGGAAAGACAA |
| | | UCCUGGACUUCCUGAAGAGCGACGGAUUCGCAAACAGAAACUUCAUGCAGCUGAU |
| | | CCACGACGACAGCCUGACAUUCAAGGAAGACAUCCAGAAGGCACAGGUCAGCGGA |
| | | CAGGGAGACAGCCUGCACGAACACAUCGCAAACCUGGCAGGAAGCCCGGCAAUCA |
| | | AGAAGGGAAUCCUGCAGACAGUCAAGGUCGUCGACGAACUGGUCAAGGUCAUGGG |
| | | AAGACACAAGCCGGAAAACAUCGUCAUCGAAAUGGCAAGAGAAAACCAGACAACA |
| | | CAGAAGGGACAGAAGAACAGCAGAGAAAGAAUGAAGAGAAUCGAAGAAGGAAUCA |
| | | AGGAACUGGGAAGCCAGAUCCUGAAGGAACACCCGGUCGAAAACACACAGCUGCA |
| | | GAACGAAAAGCUGUACCUGUACUACCUGCAGAACGGAAGAGACAUGUACGUCGAC |
| | | CAGGAACUGGACAUCAACAGACUGAGCGACUACGACGUCGACCACAUCGUCCCGC |
| | | AGAGCUUCCUGAAGGACGACAGCAUCGACAACAAGGUCCUGACAAGAAGCGACAA |
| | | GAACAGAGGAAAGAGCGACAACGUCCCGAGCGAAGAAGUCGUCAAGAAGAUGAAG |
| | | AACUACUGGAGACAGCUGCUGAACGCAAAGCUGAUCACACAGAGAAAGUUCGACA |
| | | ACCUGACAAAGGCAGAGAGAGGAGGACUGAGCGAACUGGACAAGGCAGGAUUCAU |
| | | CAAGAGACAGCUGGUCGAAACAAGACAGAUCACAAAGCACGUCGCACAGAUCCUG |
| | | GACAGCAGAAUGAACACAAAGUACGACGAAAACGACAAGCUGAUCAGAGAAGUCA |
| | | AGGUCAUCACACUGAAGAGCAAGCUGGUCAGCGACUUCAGAAAGGACUUCCAGUU |
| | | CUACAAGGUCAGAGAAAUCAACAACUACCACCACGCACACGACGCAUACCUGAAC |
| | | GCAGUCGUCGGAACAGCACUGAUCAAGAAGUACCCGAAGCUGGAAAGCGAAUUCG |
| | | UCUACGGAGACUACAAGGUCUACGACGUCAGAAAGAUGAUCGCAAAGAGCGAACA |
| | | GGAAAUCGGAAAGGCAACAGCAAAGUACUUCUUCUACAGCAACAUCAUGAACUUC |
| | | UUCAAGACAGAAAUCACACUGGCAAACGGAGAAAUCAGAAAGAGACCGCUGAUCG |
| | | AAACAAACGGAGAAACAGGAGAAAUCGUCUGGGACAAGGGAAGAGACUUCGCAAC |
| | | AGUCAGAAAGGUCCUGAGCAUGCCGCAGGUCAACAUCGUCAAGAAGACAGAAGUC |
| | | CAGACAGGAGGAUUCAGCAAGGAAAGCAUCCUGCCGAAGAGAAACAGCGACAAGC |
| | | UGAUCGCAAGAAAGAAGGACUGGGACCCGAAGAAGUACGGAGGAUUCGACAGCCC |
| | | GACAGUCGCAUACAGCGUCCUGGUCGUCGCAAAGGUCGAAAAGGGAAAGAGCAAG |
| | | AAGCUGAAGAGCGUCAAGGAACUGCUGGGAAUCACAAUCAUGGAAAGAAGCAGCU |
| | | UCGAAAAGAACCCGAUCGACUUCCUGGAAGCAAAGGGAUACAAGGAAGUCAAGAA |
| | | GGACCUGAUCAUCAAGCUGCCGAAGUACAGCCUGUUCGAACUGGAAAACGGAAGA |
| | | AAGAGAAUGCUGGCAAGCGCAGGAGAACUGCAGAAGGGAAACGAACUGGCACUGC |
| | | CGAGCAAGUACGUCAACUUCCUGUACCUGGCAAGCCACUACGAAAAGCUGAAGGG |
| | | AAGCCCGGAAGACAACGAACAGAAGCAGCUGUUCGUCGAACAGCACAAGCACUAC |
| | | CUGGACGAAAUCAUCGAACAGAUCAGCGAAUUCAGCAAGAGAGUCAUCCUGGCAG |
| | | ACGCAAACCUGGACAAGGUCCUGAGCGCAUACAACAAGCACAGAGACAAGCCGAU |
| | | CAGAGAACAGGCAGAAAACAUCAUCCACCUGUUCACACUGACAAACCUGGGAGCA |
| | | CCGGCAGCAUUCAAGUACUUCGACACAACAAUCGACAGAAAGAGAUACACAAGCA |
| | | CAAAGGAAGUCCUGGACGCAACACUGAUCCACCAGAGCAUCACAGGACUGUACGA |
| | | AACAAGAAUCGACCUGAGCCAGCUGGGAGGAGACGGAGGAGGAAGCCCGAAGAAG |
| | | AAGAGAAAGGUCUAG |
| 1007 | Amino acid | MDKKYSIGLDIGTNSVGWAVITDEYKVPSKKFKVLGNTDRHSIKKNLIGALLFDS |
| | sequence for | GETAEATRLKRTARRRYTRRKNRICYLQEIFSNEMAKVDDSFFHRLEESFLVEED |
| | Spy Cas9 | KKHERHPIFGNIVDEVAYHEKYPTIYHLRKKLVDSTDKADLRLIYLALAHMIKER |
| | | GHFLIEGDLNPDNSDVDKLFIQLVQTYNQLFEENPINASGVDAKAILSARLSKSR |
| | | RLENLIAQLPGEKKNGLFGNLIALSLGLTPNFKSNEDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPFLKDNREKI |
| | | EKILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIVD |
| | | LLEKTNRKVTVKQLKEDYFKKIECFDSVEISGVEDRENASLGTYHDLLKIIKDKD |
| | | ELDNEENEDILEDIVLTLTLFEDREMIEERLKTYAHLFDDKVMKQLKRRRYTGWG |
| | | RLSRKLINGIRDKQSGKTILDFLKSDGFANRNFMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKFDNLTKAERGGLSELDKAGFIKRQLVETRQITKHVAQIL |
| | | DSRMNTKYDENDKLIREVKVITLKSKLVSDERKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGFSKESILPKRNSDKLIARKKDWDPKKYGGEDSPTVAYSVLVVAKVEKGKSK |
| | | KLKSVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYEDTTIDRKRYTSTKEVLDATLIHQSITGLYETRIDLSQLGGDGGGSPKK |
| | | KRKV* |
| 1008 | mRNA encoding | GGGAAGCUCAGAAUAAACGCUCAACUUUGGCCGGAUCUGCCACCAUGGACAAGAA |
| | Spy Cas9 with | GUACUCCAUCGGCCUGGACAUCGGCACCAACUCCGUGGGCUGGGCCGUGAUCACC |
| | HiBiT tag | GACGAGUACAAGGUGCCCUCCAAGAAGUUCAAGGUGCUGGGCAACACCGACCGGC |
| | | ACUCCAUCAAGAAGAACCUGAUCGGCGCCCUGCUGUUCGACUCCGGCGAGACCGC |
| | | CGAGGCCACCCGGCUGAAGCGGACCGCCCGGCGGCGGUACACCCGGCGGAAGAAC |
| | | CGGAUCUGCUACCUGCAGGAGAUCUUCUCCAACGAGAUGGCCAAGGUGGACGACU |
| | | CCUUCUUCCACCGGCUGGAGGAGUCCUUCCUGGUGGAGGAGGACAAGAAGCACGA |
| | | GCGGCACCCCAUCUUCGGCAACAUCGUGGACGAGGUGGCCUACCACGAGAAGUAC |
| | | CCCACCAUCUACCACCUGCGGAAGAAGCUGGUGGACUCCACCGACAAGGCCGACC |
| | | UGCGGCUGAUCUACCUGGCCCUGGCCCACAUGAUCAAGUUCCGGGGCCACUUCCU |
| | | GAUCGAGGGCGACCUGAACCCCGACAACUCCGACGUGGACAAGCUGUUCAUCCAG |
| | | CUGGUGCAGACCUACAACCAGCUGUUCGAGGAGAACCCCAUCAACGCCUCCGGCG |
| | | UGGACGCCAAGGCCAUCCUGUCCGCCCGGCUGUCCAAGUCCCGGCGGCUGGAGAA |
| | | CCUGAUCGCCCAGCUGCCCGGCGAGAAGAAGAACGGCCUGUUCGGCAACCUGAUC |
| | | GCCCUGUCCCUGGGCCUGACCCCCAACUUCAAGUCCAACUUCGACCUGGCCGAGG |
| | | ACGCCAAGCUGCAGCUGUCCAAGGACACCUACGACGACGACCUGGACAACCUGCU |
| | | GGCCCAGAUCGGCGACCAGUACGCCGACCUGUUCCUGGCCGCCAAGAACCUGUCC |
| | | GACGCCAUCCUGCUGUCCGACAUCCUGCGGGUGAACACCGAGAUCACCAAGGCCC |
| | | CCCUGUCCGCCUCCAUGAUCAAGCGGUACGACGAGCACCACCAGGACCUGACCCU |
| | | GCUGAAGGCCCUGGUGCGGCAGCAGCUGCCCGAGAAGUACAAGGAGAUCUUCUUC |
| | | GACCAGUCCAAGAACGGCUACGCCGGCUACAUCGACGGCGGCGCCUCCCAGGAGG |
| | | AGUUCUACAAGUUCAUCAAGCCCAUCCUGGAGAAGAUGGACGGCACCGAGGAGCU |
| | | GCUGGUGAAGCUGAACCGGGAGGACCUGCUGCGGAAGCAGCGGACCUUCGACAAC |
| | | GGCUCCAUCCCCCACCAGAUCCACCUGGGCGAGCUGCACGCCAUCCUGCGGCGGC |
| | | AGGAGGACUUCUACCCCUUCCUGAAGGACAACCGGGAGAAGAUCGAGAAGAUCCU |
| | | GACCUUCCGGAUCCCCUACUACGUGGGCCCCCUGGCCCGGGGCAACUCCCGGUUC |
| | | GCCUGGAUGACCCGGAAGUCCGAGGAGACCAUCACCCCCUGGAACUUCGAGGAGG |
| | | UGGUGGACAAGGGCGCCUCCGCCCAGUCCUUCAUCGAGCGGAUGACCAACUUCGA |
| | | CAAGAACCUGCCCAACGAGAAGGUGCUGCCCAAGCACUCCCUGCUGUACGAGUAC |
| | | UUCACCGUGUACAACGAGCUGACCAAGGUGAAGUACGUGACCGAGGGCAUGCGGA |
| | | AGCCCGCCUUCCUGUCCGGCGAGCAGAAGAAGGCCAUCGUGGACCUGCUGUUCAA |
| | | GACCAACCGGAAGGUGACCGUGAAGCAGCUGAAGGAGGACUACUUCAAGAAGAUC |
| | | GAGUGCUUCGACUCCGUGGAGAUCUCCGGCGUGGAGGACCGGUUCAACGCCUCCC |
| | | UGGGCACCUACCACGACCUGCUGAAGAUCAUCAAGGACAAGGACUUCCUGGACAA |
| | | CGAGGAGAACGAGGACAUCCUGGAGGACAUCGUGCUGACCCUGACCCUGUUCGAG |
| | | GACCGGGAGAUGAUCGAGGAGCGGCUGAAGACCUACGCCCACCUGUUCGACGACA |
| | | AGGUGAUGAAGCAGCUGAAGCGGCGGCGGUACACCGGCUGGGGCCGGCUGUCCCG |
| | | GAAGCUGAUCAACGGCAUCCGGGACAAGCAGUCCGGCAAGACCAUCCUGGACUUC |
| | | CUGAAGUCCGACGGCUUCGCCAACCGGAACUUCAUGCAGCUGAUCCACGACGACU |
| | | CCCUGACCUUCAAGGAGGACAUCCAGAAGGCCCAGGUGUCCGGCCAGGGCGACUC |
| | | CCUGCACGAGCACAUCGCCAACCUGGCCGGCUCCCCCGCCAUCAAGAAGGGCAUC |
| | | CUGCAGACCGUGAAGGUGGUGGACGAGCUGGUGAAGGUGAUGGGCCGGCACAAGC |
| | | CCGAGAACAUCGUGAUCGAGAUGGCCCGGGAGAACCAGACCACCCAGAAGGGCCA |
| | | GAAGAACUCCCGGGAGCGGAUGAAGCGGAUCGAGGAGGGCAUCAAGGAGCUGGGC |
| | | UCCCAGAUCCUGAAGGAGCACCCCGUGGAGAACACCCAGCUGCAGAACGAGAAGC |
| | | UGUACCUGUACUACCUGCAGAACGGCCGGGACAUGUACGUGGACCAGGAGCUGGA |
| | | CAUCAACCGGCUGUCCGACUACGACGUGGACCACAUCGUGCCCCAGUCCUUCCUG |
| | | AAGGACGACUCCAUCGACAACAAGGUGCUGACCCGGUCCGACAAGAACCGGGGCA |
| | | AGUCCGACAACGUGCCCUCCGAGGAGGUGGUGAAGAAGAUGAAGAACUACUGGCG |
| | | GCAGCUGCUGAACGCCAAGCUGAUCACCCAGCGGAAGUUCGACAACCUGACCAAG |
| | | GCCGAGCGGGGCGGCCUGUCCGAGCUGGACAAGGCCGGCUUCAUCAAGCGGCAGC |
| | | UGGUGGAGACCCGGCAGAUCACCAAGCACGUGGCCCAGAUCCUGGACUCCCGGAU |
| | | GAACACCAAGUACGACGAGAACGACAAGCUGAUCCGGGAGGUGAAGGUGAUCACC |
| | | CUGAAGUCCAAGCUGGUGUCCGACUUCCGGAAGGACUUCCAGUUCUACAAGGUGC |
| | | GGGAGAUCAACAACUACCACCACGCCCACGACGCCUACCUGAACGCCGUGGUGGG |
| | | CACCGCCCUGAUCAAGAAGUACCCCAAGCUGGAGUCCGAGUUCGUGUACGGCGAC |
| | | UACAAGGUGUACGACGUGCGGAAGAUGAUCGCCAAGUCCGAGCAGGAGAUCGGCA |
| | | AGGCCACCGCCAAGUACUUCUUCUACUCCAACAUCAUGAACUUCUUCAAGACCGA |
| | | GAUCACCCUGGCCAACGGCGAGAUCCGGAAGCGGCCCCUGAUCGAGACCAACGGC |
| | | GAGACCGGCGAGAUCGUGUGGGACAAGGGCCGGGACUUCGCCACCGUGCGGAAGG |
| | | UGCUGUCCAUGCCCCAGGUGAACAUCGUGAAGAAGACCGAGGUGCAGACCGGCGG |
| | | CUUCUCCAAGGAGUCCAUCCUGCCCAAGCGGAACUCCGACAAGCUGAUCGCCCGG |
| | | AAGAAGGACUGGGACCCCAAGAAGUACGGCGGCUUCGACUCCCCCACCGUGGCCU |
| | | ACUCCGUGCUGGUGGUGGCCAAGGUGGAGAAGGGCAAGUCCAAGAAGCUGAAGUC |
| | | CGUGAAGGAGCUGCUGGGCAUCACCAUCAUGGAGCGGUCCUCCUUCGAGAAGAAC |
| | | CCCAUCGACUUCCUGGAGGCCAAGGGCUACAAGGAGGUGAAGAAGGACCUGAUCA |
| | | UCAAGCUGCCCAAGUACUCCCUGUUCGAGCUGGAGAACGGCCGGAAGCGGAUGCU |
| | | GGCCUCCGCCGGCGAGCUGCAGAAGGGCAACGAGCUGGCCCUGCCCUCCAAGUAC |
| | | GUGAACUUCCUGUACCUGGCCUCCCACUACGAGAAGCUGAAGGGCUCCCCCGAGG |
| | | ACAACGAGCAGAAGCAGCUGUUCGUGGAGCAGCACAAGCACUACCUGGACGAGAU |
| | | CAUCGAGCAGAUCUCCGAGUUCUCCAAGCGGGUGAUCCUGGCCGACGCCAACCUG |
| | | GACAAGGUGCUGUCCGCCUACAACAAGCACCGGGACAAGCCCAUCCGGGAGCAGG |
| | | CCGAGAACAUCAUCCACCUGUUCACCCUGACCAACCUGGGCGCCCCCGCCGCCUU |
| | | CAAGUACUUCGACACCACCAUCGACCGGAAGCGGUACACCUCCACCAAGGAGGUG |
| | | CUGGACGCCACCCUGAUCCACCAGUCCAUCACCGGCCUGUACGAGACCCGGAUCG |
| | | ACCUGUCCCAGCUGGGCGGCGACGGCGGCGGCUCCCCCAAGAAGAAGCGGAAGGU |
| | | GUCCGAGUCCGCCACCCCCGAGUCCGUGUCCGGCUGGCGGCUGUUCAAGAAGAUC |
| | | UCCUGACUAGCACCAGCCUCAAGAACACCCGAAUGGAGUCUCUAAGCUACAUAAU |
| | | ACCAACUUACACUUUACAAAAUGUUGUCCCCCAAAAUGUAGCCAUUCGUAUCUGC |
| | | UCCUAAUAAAAAGAAAGUUUCUUCACAUUCUCUCGAGAAAAAAAAAAAAAAAAAA |
| | | AAAAAAAAAAAAAAAAAAAAAAAAAAAAAAAAAAAAAAAAAAAAAAAAAAAAAAA |
| | | AAAAAAAAAAAAAAAAAAAAUCUAG |
| 1009 | Open reading | auggacaagaaguacuccaucggccuggacaucggcaccaacuccgugggcuggg |
| | frame for Spy | ccgugaucaccgacgaguacaaggugcccuccaagaaguucaaggugcugggcaa |
| | Cas9 with | caccgaccggcacuccaucaagaagaaccugaucggcgcccugcuguucgacucc |
| | HiBiT tag | ggcgagaccgccgaggccacccggcugaagcggaccgcccggcggcgguacaccc |
| | | ggcggaagaaccggaucugcuaccugcaggagaucuucuccaacgagauggccaa |
| | | gguggacgacuccuucuuccaccggcuggaggaguccuuccugguggaggaggac |
| | | aagaagcacgagcggcaccccaucuucggcaacaucguggacgagguggccuacc |
| | | acgagaaguaccccaccaucuaccaccugcggaagaagcugguggacuccaccga |
| | | caaggccgaccugcggcugaucuaccuggcccuggcccacaugaucaaguuccgg |
| | | ggccacuuccugaucgagggcgaccugaaccccgacaacuccgacguggacaagc |
| | | uguucauccagcuggugcagaccuacaaccagcuguucgaggagaaccccaucaa |
| | | cgccuccggcguggacgccaaggccauccuguccgcccggcuguccaagucccgg |
| | | cggcuggagaaccugaucgcccagcugcccggcgagaagaagaacggccuguucg |
| | | gcaaccugaucgcccugucccugggccugacccccaacuucaaguccaacuucga |
| | | ccuggccgaggacgccaagcugcagcuguccaaggacaccuacgacgacgaccug |
| | | gacaaccugcuggcccagaucggcgaccaguacgccgaccuguuccuggccgcca |
| | | agaaccuguccgacgccauccugcuguccgacauccugcgggugaacaccgagau |
| | | caccaaggccccccuguccgccuccaugaucaagcgguacgacgagcaccaccag |
| | | gaccugacccugcugaaggcccuggugcggcagcagcugcccgagaaguacaagg |
| | | agaucuucuucgaccaguccaagaacggcuacgccggcuacaucgacggcggcgc |
| | | cucccaggaggaguucuacaaguucaucaagcccauccuggagaagauggacggc |
| | | accgaggagcugcuggugaagcugaaccgggaggaccugcugcggaagcagcgga |
| | | ccuucgacaacggcuccaucccccaccagauccaccugggcgagcugcacgccau |
| | | ccugcggcggcaggaggacuucuaccccuuccugaaggacaaccgggagaagauc |
| | | gagaagauccugaccuuccggauccccuacuacgugggcccccuggcccggggca |
| | | acucccgguucgccuggaugacccggaaguccgaggagaccaucacccccuggaa |
| | | cuucgaggaggugguggacaagggcgccuccgcccaguccuucaucgagcggaug |
| | | accaacuucgacaagaaccugcccaacgagaaggugcugcccaagcacucccugc |
| | | uguacgaguacuucaccguguacaacgagcugaccaaggugaaguacgugaccga |
| | | gggcaugcggaagcccgccuuccuguccggcgagcagaagaaggccaucguggac |
| | | cugcuguucaagaccaaccggaaggugaccgugaagcagcugaaggaggacuacu |
| | | ucaagaagaucgagugcuucgacuccguggagaucuccggcguggaggaccgguu |
| | | caacgccucccugggcaccuaccacgaccugcugaagaucaucaaggacaaggac |
| | | uuccuggacaacgaggagaacgaggacauccuggaggacaucgugcugacccuga |
| | | cccuguucgaggaccgggagaugaucgaggagcggcugaagaccuacgcccaccu |
| | | guucgacgacaaggugaugaagcagcugaagcggcggcgguacaccggcuggggc |
| | | cggcugucccggaagcugaucaacggcauccgggacaagcaguccggcaagacca |
| | | uccuggacuuccugaaguccgacggcuucgccaaccggaacuucaugcagcugau |
| | | ccacgacgacucccugaccuucaaggaggacauccagaaggcccagguguccggc |
| | | cagggcgacucccugcacgagcacaucgccaaccuggccggcucccccgccauca |
| | | agaagggcauccugcagaccgugaaggugguggacgagcuggugaaggugauggg |
| | | ccggcacaagcccgagaacaucgugaucgagauggcccgggagaaccagaccacc |
| | | cagaagggccagaagaacucccgggagcggaugaagcggaucgaggagggcauca |
| | | aggagcugggcucccagauccugaaggagcaccccguggagaacacccagcugca |
| | | gaacgagaagcuguaccuguacuaccugcagaacggccgggacauguacguggac |
| | | caggagcuggacaucaaccggcuguccgacuacgacguggaccacaucgugcccc |
| | | aguccuuccugaaggacgacuccaucgacaacaaggugcugacccgguccgacaa |
| | | gaaccggggcaaguccgacaacgugcccuccgaggagguggugaagaagaugaag |
| | | aacuacuggcggcagcugcugaacgccaagcugaucacccagcggaaguucgaca |
| | | accugaccaaggccgagcggggcggccuguccgagcuggacaaggccggcuucau |
| | | caagcggcagcugguggagacccggcagaucaccaagcacguggcccagauccug |
| | | gacucccggaugaacaccaaguacgacgagaacgacaagcugauccgggagguga |
| | | aggugaucacccugaaguccaagcugguguccgacuuccggaaggacuuccaguu |
| | | cuacaaggugcgggagaucaacaacuaccaccacgcccacgacgccuaccugaac |
| | | gccguggugggcaccgcccugaucaagaaguaccccaagcuggaguccgaguucg |
| | | uguacggcgacuacaagguguacgacgugcggaagaugaucgccaaguccgagca |
| | | ggagaucggcaaggccaccgccaaguacuucuucuacuccaacaucaugaacuuc |
| | | uucaagaccgagaucacccuggccaacggcgagauccggaagcggccccugaucg |
| | | agaccaacggcgagaccggcgagaucgugugggacaagggccgggacuucgccac |
| | | cgugcggaaggugcuguccaugccccaggugaacaucgugaagaagaccgaggug |
| | | cagaccggcggcuucuccaaggaguccauccugcccaagcggaacuccgacaagc |
| | | ugaucgcccggaagaaggacugggaccccaagaaguacggcggcuucgacucccc |
| | | caccguggccuacuccgugcuggugguggccaagguggagaagggcaaguccaag |
| | | aagcugaaguccgugaaggagcugcugggcaucaccaucauggagcgguccuccu |
| | | ucgagaagaaccccaucgacuuccuggaggccaagggcuacaaggaggugaagaa |
| | | ggaccugaucaucaagcugcccaaguacucccuguucgagcuggagaacggccgg |
| | | aagcggaugcuggccuccgccggcgagcugcagaagggcaacgagcuggcccugc |
| | | ccuccaaguacgugaacuuccuguaccuggccucccacuacgagaagcugaaggg |
| | | cucccccgaggacaacgagcagaagcagcuguucguggagcagcacaagcacuac |
| | | cuggacgagaucaucgagcagaucuccgaguucuccaagcgggugauccuggccg |
| | | acgccaaccuggacaaggugcuguccgccuacaacaagcaccgggacaagcccau |
| | | ccgggagcaggccgagaacaucauccaccuguucacccugaccaaccugggcgcc |
| | | cccgccgccuucaaguacuucgacaccaccaucgaccggaagcgguacaccucca |
| | | ccaaggaggugcuggacgccacccugauccaccaguccaucaccggccuguacga |
| | | gacccggaucgaccugucccagcugggcggcgacggcggcggcucccccaagaag |
| | | aagcggaagguguccgaguccgccacccccgaguccguguccggcuggcggcugu |
| | | ucaagaagaucuccuga |
| 1010 | Amino acid | MDKKYSIGLDIGTNSVGWAVITDEYKVPSKKFKVLGNTDRHSIKKNLIGALLFDS |
| | sequence for | GETAEATRLKRTARRRYTRRKNRICYLQEIFSNEMAKVDDSFFHRLEESFLVEED |
| | Spy Cas9 with | KKHERHPIFGNIVDEVAYHEKYPTIYHLRKKLVDSTDKADLRLIYLALAHMIKFR |
| | HiBiT tag | GHFLIEGDLNPDNSDVDKLFIQLVQTYNQLFEENPINASGVDAKAILSARLSKSR |
| | | RLENLIAQLPGEKKNGLFGNLIALSLGLTPNEKSNEDLAEDAKLQLSKDTYDDDL |
| | | DNLLAQIGDQYADLFLAAKNLSDAILLSDILRVNTEITKAPLSASMIKRYDEHHQ |
| | | DLTLLKALVRQQLPEKYKEIFFDQSKNGYAGYIDGGASQEEFYKFIKPILEKMDG |
| | | TEELLVKLNREDLLRKQRTEDNGSIPHQIHLGELHAILRRQEDFYPFLKDNREKI |
| | | EKILTFRIPYYVGPLARGNSRFAWMTRKSEETITPWNFEEVVDKGASAQSFIERM |
| | | TNFDKNLPNEKVLPKHSLLYEYFTVYNELTKVKYVTEGMRKPAFLSGEQKKAIVD |
| | | LLFKTNRKVTVKQLKEDYFKKIECFDSVEISGVEDRFNASLGTYHDLLKIIKDKD |
| | | ELDNEENEDILEDIVLTLTLFEDREMIEERLKTYAHLEDDKVMKQLKRRRYTGWG |
| | | RLSRKLINGIRDKQSGKTILDELKSDGFANRNFMQLIHDDSLTFKEDIQKAQVSG |
| | | QGDSLHEHIANLAGSPAIKKGILQTVKVVDELVKVMGRHKPENIVIEMARENQTT |
| | | QKGQKNSRERMKRIEEGIKELGSQILKEHPVENTQLQNEKLYLYYLQNGRDMYVD |
| | | QELDINRLSDYDVDHIVPQSFLKDDSIDNKVLTRSDKNRGKSDNVPSEEVVKKMK |
| | | NYWRQLLNAKLITQRKEDNLTKAERGGLSELDKAGFIKRQLVETRQITKHVAQIL |
| | | DSRMNTKYDENDKLIREVKVITLKSKLVSDERKDFQFYKVREINNYHHAHDAYLN |
| | | AVVGTALIKKYPKLESEFVYGDYKVYDVRKMIAKSEQEIGKATAKYFFYSNIMNF |
| | | FKTEITLANGEIRKRPLIETNGETGEIVWDKGRDFATVRKVLSMPQVNIVKKTEV |
| | | QTGGFSKESILPKRNSDKLIARKKDWDPKKYGGEDSPTVAYSVLVVAKVEKGKSK |
| | | KLKSVKELLGITIMERSSFEKNPIDFLEAKGYKEVKKDLIIKLPKYSLFELENGR |
| | | KRMLASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLTNLGA |
| | | PAAFKYFDTTIDRKRYTSTKEVLDATLIHQSITGLYETRIDLSQLGGDGGGSPKK |
| | | KRKVSESATPESVSGWRLFKKIS* |
| 1011 | Exemplary EMX1 | mG*mA*mG*UCCGAGCAGAAGAAGAAGUUUUAGAmGmCmUmAmGmAmAmAmUmAm |
| | guide RNA | GmCAAGUUAAAAUAAGGCUAGUCCGUUAUCAmAmCmUmUmGmAmAmAmAmAmGmU |
| | (G000644) | mGmGmCmAmCmCmGmAmGmUmCmGmGmUmGmCmU*mU*mU*mU |
| 1012 | Exemplary | mG*mA*mC*CCCCUCCACCCCGCCUCGUUUUAGAmGmCmUmAmGmAmAmAmUmAm |
| | VEGFA guide | GmCAAGUUAAAAUAAGGCUAGUCCGUUAUCAmAmCmUmUmGmAmAmAmAmAmGmU |
| | RNA (G000645) | mGmGmCmAmCmCmGmAmGmUmCmGmGmUmGmCmU*mU*mU*mU |
| 1013 | Exemplary SV40 | PKKKRKV |
| | NLS | |
| 1014 | Exemplary SV40 | PKKKRRV |
| | NLS | |
| 1015 | Exemplary | KRPAATKKAGQAKKKK |
| | nucleoplasmin | |
| | NLS | |

What is claimed is:

1. A guide RNA comprising:
A. a targeting sequence comprising a sequence at least 95%, 90%, 85%, or 80% identical to or complementary to the nucleotide sequence of SEQ ID NOs: 9, 1, 2, 7, 13-15, 17, 18, or 20;
B. a targeting sequence comprising a sequence identical to or complementary to at least 17, 18, 19, or 20 contiguous nucleotides of the nucleotide sequence of SEQ ID NOs: 9, 1, 2, 7, 13-15, 17, 18, or 20; or
C. a targeting sequence comprising a targeting sequence identical to the nucleotide sequence of SEQ ID NOs: 9, 1, 2, 7, 13-15, 17, 18, or 20.

2. The guide of claim 1, comprising a sequence a targeting sequence identical to the nucleotide sequence of SEQ ID NOs: 9, 14, or 18.

3. The guide RNA of claim 1 or 2, further comprising one or more of:
A. a shortened hairpin 1 region, or a substituted and optionally shortened hairpin 1 region, wherein
1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
a. any one or two of H1-5 through H1-8,
b. one, two, or three of the following pairs of nucleotides: H1-1 and H1-12, H1-2 and H1-11, H1-3 and H1-10, and H1-4 and H1-9, or
c. 1-8 nucleotides of hairpin 1 region; or
2. the shortened hairpin 1 region lacks 4-8 nucleotides, preferably 4-6 nucleotides; and
a one or more of positions H1-1, H1-2, or H1-3 is deleted or substituted relative to Exemplary SpyCas9 sgRNA-1; or
b. one or more of positions H1-6 through H1-10 is substituted relative to Exemplary SpyCas9 sgRNA-1; or
3. the shortened hairpin 1 region lacks 5-10 nucleotides, preferably 5-6 nucleotides, and one or more of positions N18, H1-12, or n is substituted relative to Exemplary SpyCas9 sgRNA-1; or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1; or
C. a substitution relative to Exemplary SpyCas9 sgRNA—at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. an Exemplary SpyCas9 sgRNA-1 with an upper stem region, wherein
the upper stem modification comprises a modification to any one or more of US1-US12 in the upper stem region.

4. The guide RNA of claim 3, wherein the guide RNA lacks 6 nucleotides in shortened hairpin 1.

5. The guide RNA of claim 3, wherein the guide RNA lacks 8 nucleotides in shortened hairpin 1.

6. The guide RNA of any one of claims 3-5, wherein H-1 and H-3 are deleted.

7. The guide RNA of any one of claims 3-6, wherein the guide RNA further comprises a 3′ tail.

8. The guide RNA of claim 7, wherein the 3′ tail is 1˜4 nucleotides in length, optionally 1 nucleotide in length.

9. The guide RNA of any one of claims 3-8, wherein the guide RNA comprises an upper stem region comprising a modification to any one or more of US1-US12 in the upper stem region.

10. The guide RNA of claim 1 or 2, comprising a modified nucleotide sequence according to the pattern (mN*)3(N)13-17, wherein “m” is indicative of a 2′-O-methyl modification, * is indicative of a phosphorothioate bond, and N is indicative of a 2′-OH and a phosphodiester bond.

11. The guide RNA of claim 1, wherein the guide RNA comprises a modified nucleotide sequence selected from a sequence in Table 4A (SEQ ID NO: 501-512, optionally SEQ ID NO: 507 or 512), wherein the modified nucleotide sequence is 3′ of the guide sequence.

12. The guide RNA of claim 11, modified according to the pattern of nucleotide sequence selected from a sequence in Table 4B (SEQ ID NO: 601-612, optionally SEQ ID NO: 607 or 612), wherein the (mN*)3N17 refers to the targeting sequence of claim 1 or 2.

13. The guide RNA of any one of claims 1-12, wherein the guide RNA comprises the nucleotide sequence selected from SEQ ID NOs: 121, 109, 101, 102, 107, 113-115, 117, 118, 120, 122, or 123, optionally SEQ ID NOs: 109, 114, 118, 121, 122, or 123 as provided in Table 2.

14. The guide RNA of claim 13, wherein each nucleotide is any natural or non-natural nucleotide.

15. The guide RNA of claim 14, wherein the guide RNA comprises the modified nucleotide sequence selected from SEQ ID Nos: 221, 209, 201, 202, 207, 213-215, 217, 218, 220, 222, or 223, optionally SEQ ID NOs: 209, 214, 218, 221, 222, or 223 as provided in Table 2.

16. A composition comprising a guide RNA of any one of claims 1-15.

17. The composition of claim 16, further comprising an RNA-guided DNA binding agent or a nucleic acid encoding an RNA-guided DNA binding agent.

18. The composition of claim 17, wherein the nucleic acid encoding the RNA-guided DNA binding agent comprises an mRNA comprising an open reading frame (ORF) encoding the RNA-guided DNA binding agent.

19. The composition of claim 17 or 18, wherein the RNA-guided DNA binding agent is a Cas9 nuclease.

20. The composition of claim 19, wherein the Cas9 is S. pyogenes Cas9.

21. The composition of claim 20, wherein the S. pyogenes Cas9 comprises an amino acid sequence having at least 90% identity to SEQ ID NOs: 1001, 1004, 1007, or 1010, or an ORF encoding a S. pyogenes Cas9 having at least 90% identity to a sequence selected from SEQ ID NOs: 1003, 1006, and 1009.

22. The composition of claim 21, wherein the ORF encoding the amino acid sequence has at least 95% identity to SEQ ID NOs: 1003, 1006, or 1009.

23. The composition of any one of claims 19-22, wherein the nuclease has double-stranded endonuclease activity.

24. The composition of any one of claims 18-23, wherein the ORF is a modified ORF.

25. The composition of claim 21, wherein the guide RNA comprises a targeting sequence identical to the nucleotide sequence of SEQ ID NO: 9 and the S. pyogenes Cas9 comprises an amino acid sequence having at least 95% identity to SEQ ID NOs: 1001, wherein the S. pyogenes Cas9 wherein the nuclease has double stranded endonuclease activity.

26. The composition of claim 21, wherein the guide RNA comprises a targeting sequence comprising a sequence identical to the nucleotide sequence of SEQ ID NO: 9 and the S. pyogenes Cas9 comprises an amino acid sequence comprising the amino acid sequence of SEQ ID NOs: 1001.

27. The composition of claim 21, wherein the guide RNA comprises a targeting sequence comprising a sequence identical to the nucleotide sequence of SEQ ID NO: 9 and wherein the S. pyogenes Cas9 an ORF encoding a S. pyogenes Cas9 having at least 90% identity to a sequence selected from SEQ ID NOs: 1003, wherein the S. pyogenes Cas9 wherein the nuclease has double stranded endonuclease activity.

28. The composition of any one of claims 25-27, wherein the ORF is a modified ORF.

29. The composition of any one of claims 25-28, wherein the guide RNA comprises the nucleotide sequence of SEQ ID NO: 121 or 109.

30. The composition of any one of claims 25-28, wherein the guide RNA comprises the modified nucleotide sequence of SEQ ID NO: 221 or 209.

31. The composition of any one of claims 16-30, further comprising a pharmaceutical excipient.

32. The composition of any one of claims 16-31, wherein the guide RNA is associated with a lipid nanoparticle (LNP).

33. The composition of claim 32, wherein the LNP comprises a cationic lipid.

34. The composition of claim 33, wherein the cationic lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate.

35. The composition of any one of claims 32-34, wherein the LNP comprises a helper lipid.

36. The composition of claim 35, wherein the helper lipid is cholesterol.

37. The composition of any one of claims 32-36, wherein the LNP comprises a neutral lipid.

38. The composition of claim 37, wherein the neutral lipid is 1,2-distearoyl-sn-glycero-3-phosphocholine (DSPC).

39. The composition of any one of claims 32-38, wherein the LNP comprises a stealth lipid.

40. The composition of claim 39, wherein the stealth lipid is 1,2-dimyristoyl-rac-glycero-3-methoxypolyethylene glycol-2000 (PEG2k-DMG).

41. The composition of claim 32, wherein the LNP comprises (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate, DSPC, cholesterol, and PEG2k-DMG.

42. A pharmaceutical composition comprising the guide RNA of any one of claims 1-15 or the composition of any one of claims 16-41.

43. A pharmaceutical composition comprising, or use of, the guide RNA of any one of claims 1-15 or the composition of any one of claims 16-41 for inducing a double-strand break or a single-strand break within a PCSK9 gene in a cell or reducing expression of a PCSK9 gene in a cell.

44. The pharmaceutical composition or use of claim 43, wherein the cell is a liver cell.

45. The pharmaceutical composition or use of claim 44, wherein the cell is in a subject.

46. A pharmaceutical composition comprising, or use of, the guide RNA of any one of claims 1-15 or the composition of any one of claims 16-41 for treating a subject having a PCSK9 related disease.

47. A method of inducing a double-strand break or a single-strand break within a PCSK9 gene in a cell or reducing expression of a PCSK9 protein in a cell comprising contacting a cell with the guide RNA of any one of claims 1-15 and an RNA-guided DNA binding agent or a nucleic acid encoding an RNA-guided DNA binding agent, or the composition of any one of claims 16-41.

48. Use of the guide RNA of any one of claims 1-15 or the composition of any one of claims 16-41 in the preparation of a medicament for practicing the method of claim 47.

49. A human liver cell comprising an indel in a nucleotide sequence selected from a genomic locus in Table 1.

50. The human liver cell of claim 49, comprising an indel in a nucleotide sequence selected from a genomic locus selected from the genomic locus of SEQ ID NO: 9, 1, 2, 7, 13-15, 17, 18, or 20.

51. A method of modifying a genomic locus in a human liver cell, the method comprising contacting a human liver cell with the guide RNA of any one of claims 1-15 and an RNA-guided DNA binding agent or a nucleic acid encoding an RNA-guided DNA binding agent, or the composition of any one of claims 16-41.

52. The method of claim 51, wherein the method is performed in vivo.

53. The pharmaceutical composition, method, or cell of any one of claims 44, 45, 49-52, wherein the liver cell is a hepatocyte.

54. The pharmaceutical composition, method, or cell of claim 53, wherein the cell is in a subject with a PCSK9 related disease.

55. A method of treating a PCSK9 related disease in a subject, the method comprising administering to the subject the guide RNA of any one of claims 1-15 and an RNA-guided DNA binding agent or a nucleic acid encoding an RNA-guided DNA binding agent, or the composition of any one of claims 16-41, or the pharmaceutical composition of claim 42.

56. The pharmaceutical composition, method, or cell of any one of claims 42-55, further comprising determining the PCSK9 protein level in a subject blood or serum sample.

57. Use of the guide RNA of any one of claims 1-15 or the composition of any 57. one of claims 16-41, or the pharmaceutical composition of claim 42 in the preparation of a medicament for practicing any of the methods of claim 47 or 51-56.

58. A kit comprising the guide RNA of any one of claims 1-15 and an RNA-guided DNA binding agent or a nucleic acid encoding an RNA-guided DNA binding agent, the composition of any one of claims 16-41, or the pharmaceutical composition of any one of claims 42-46.

59. A kit for use or for practicing the method of any one of claim 47 or 51-56.
